(19) United States
(12) Patent Application Publication
(10) Pub. No.: US 2024/0350622 A1
(43) Pub. Date: Oct. 24, 2024

(54) Vaccine Antigen

(71) Applicant: Macfarlane Burnet Institute for Medical Research and Public Health Limited, Melbourne (AU)

(72) Inventors: Pantelis Poumbourios, Melbourne (AU); Heidi Drummer, Melbourne (AU)

(21) Appl. No.: 18/682,740

(22) PCT Filed: Aug. 11, 2022
(86) PCT No.: PCT/AU2022/050880

§ 371 Date: Feb. 9, 2024

Related U.S. Application Data
(63) Continuation of application No. PCT/AU2022/050429, filed on May 6, 2022.

(30) Foreign Application Priority Data
Aug. 11, 2021 (AU) 2021902500
Aug. 13, 2021 (AU) 2021902530

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/55505; A61K 2039/55555; A61K 2039/55566; A61K 2039/575; C12N 2770/20022; C12N 2770/20034; C12N 2770/20051

(57) ABSTRACT

The field of the specification relates broadly to SARS-CoV-2 vaccine spike protein antigens and methods of using and manufacturing these antigens. The invention also relates to vectors and polynucleotides encoding the SARS-CoV-2 vaccine antigens and vaccines, kits, devices and strips comprising the coronavirus vaccine antigen. The spike protein from SARS-CoV-2 has prolines substituted at positions 986, 987 (2P or S-2P) and additional alanine cavity filling mutations at positions A1016 and A1020.

### FIELD

The field of the specification relates broadly to coronavirus vaccine antigens and methods of using and manufacturing coronavirus vaccine antigens. The invention also relates to vectors and polynucleotides encoding the coronavirus vaccine antigens and vaccines, kits, devices and strips comprising the coronavirus vaccine antigen.

### BACKGROUND

Bibliographic details of references in the subject specification are also listed at the end of the specification.

Reference to any prior art in this specification is not, and should not be taken as, acknowledgement or any form of suggestion that this prior art forms part of the common general knowledge in any country.

The SARS-CoV-2 has killed more than 4 million people globally with a strong age dependent infection fatality rate. First-generation vaccines that deliver ancestral SARS-CoV-2 derived viral Spike glycoprotein(S) sequences for in vivo expression and neutralizing antibody (NAb) induction have proven highly effective at preventing symptomatic and severe COVID-19 and have been rolled out across the globe. Highly effective S vaccine platforms include mRNA (Pfizer-BioNTech, BNT162b2; Moderna mRNA-1273) (Baden et al., 2021; Polack et al., 2020), adenovirus 26 and adenovirus 5 (Ad26, Ad5) (Sputnik V; Janssen COVID-19) (Logunov et al., 2021; Sadoff et al., 2021) and chimpanzee adenovirus (Astrazeneca/Oxford, ChAdOx1 nCoV-19) (Emary et al., 2021; Madhi et al., 2021; Voysey et al., 2021). The sites of vulnerability to neutralizing antibody (NAb) within S are being revealed with the isolation of monoclonal NAbs from COVID-19 patients. The ACE2 receptor binding domain (RBD) of S is an immunodominant antibody target in natural infection and highly potent NAbs directed to the RBD can block infection via ACE2 receptor mimicry (e.g. B38), or via steric blockade of ACE2 binding (e.g. H4) or by binding to a quaternary epitope formed by 2 RBD monomers (e.g. 2-43). The N-terminal domain (NTD) of S1 has been identified as a supersite of vulnerability and comprises multiple antigenic sites (Andreano et al., 2020; Cerutti et al., 2021; Liu, et al., 2020; McCallum et al., 2021). This region exhibits a high degree of plasticity acquiring deletions, insertions and glycan additions in order to evade antibody. Undefined neutralization epitopes have also been observed within S1 and S2 (Brouwer et al., 2020; Jennewein et al., 2021).

Naturally acquired immunity to SARS-CoV-2 is believed to be driving the emergence of variants of concern (VOCs) with mutations in the RBD and NTD that reduce the neutralization potency of convalescent and vaccine-induced immune sera as well as human monoclonal NAbs (Plante et al., 2021). Key mutations observed in the RBD of VOCs include K417T/N, N439K, L452R, Y453F, S477N, T478K, E484K/Q, and N501Y, while in the NTD, deletion of amino acids 69-70, 156-157 and 242-245 have been observed. Vaccine efficacy can also vary with VOCs depending on viral S sequence and vaccine modality. Thus, ChAdOx1-nCOV-19 vaccine efficacy was reduced from 81.5% to 70.4% in the case of the alpha/B.1.1.7 isolate (Emary et al., 2021), whereas efficacy was reduced to 10.4% for the beta/B.1.351 isolate (Madhi et al., 2021). The reduced efficacy of ChAdOx1 nCOV-19 against these VOCs appears to correlate with reduced in vitro neutralization potency of vaccine sera against the VOCs (Dejnirattisai et al., 2021; Supasa et al., 2021). The neutralization potency of mRNA vaccinee sera is also diminished for VOCs (Alter et al., 2021; Dejnirattisai et al., 2021; Garcia-Beltran et al., 2021; Liu et al., 2021; Supasa et al., 2021; Tada et al., 2021). However, full vaccination with BNT162b2 provides a high level of protection against infection and disease caused by the alpha and beta variants (Abu-Raddad et al., 2021), although where break-through infections have occurred, these have been found to be associated with VOCs (Kustin et al., 2021). While current vaccines have thus far retained substantial efficacy against most VOCs, boosting with VOC matched vaccines may be required to maintain immunity against emerging viral variants as the pandemic evolves in a setting of a partially immune human population.

Thus, there is a need for improved antigens for eliciting immune responses to coronaviruses. In particular, there is a need for improved antigens for eliciting immune responses to coronavirus VOCs.

### SUMMARY OF THE DISCLOSURE

The term “and/or”, e.g., “X and/or Y” shall be understood to mean either “X and Y” or “X or Y” and shall be taken to provide explicit support for both meanings or for either meaning. As used herein, the term “about”, unless stated to the contrary, refers to +/−10%, more preferably +/−5%, even more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise. Each embodiment in this specification is to be applied mutatis mutandis to every other embodiment unless expressly stated otherwise.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A sequence listing is provided after the claims.

In an aspect, the present invention provides a coronavirus vaccine antigen comprising a coronavirus S protein trimer wherein the S protein trimer is modified to comprise a structural modification which reduces the size of the alanine cavity within the coiled-coil region of the S protein trimer and wherein the S protein trimer elicits neutralising antibody responses.

In an aspect, the present invention provides a coronavirus vaccine antigen comprising a coronavirus S protein trimer wherein at least one amino acid in the region of the S protein monomer forming the coiled-coil of the S protein trimer is substituted with a more hydrophobic amino acid.

In an aspect, the present invention provides a vector or polynucleotide encoding the S protein monomer of the coronavirus (CoV) vaccine antigen as described herein. In one embodiment, polynucleotides are codon optimised for expression in a host cell or a vaccine recipient cell.

In one embodiment, the present invention provides a polynucleotide complement of the polynucleotide encoding the S protein monomer of the coronavirus (CoV) vaccine antigen as described herein.

In an aspect, the present invention provides a host cell comprising the vector or polynucleotide as described herein. In one embodiment, the host cell is a host cell for in vitro expression not a vaccine recipient cell.

In an aspect, the present invention provides a method of producing the coronavirus (CoV) vaccine antigen as described herein comprising culturing the host cell as described herein in culture medium.

In an aspect, the present invention provides a protein nanoparticle comprising the coronavirus (CoV) vaccine antigen as described herein.

In an aspect, the present invention provides a virus-like particle comprising the coronavirus (CoV) vaccine antigen as described herein.

In an aspect, the present invention provides a vial or a solid or semi-solid surface containing the vector or polynucleotide, antigen, protein nanoparticle, or VLP as described herein.

In an aspect, the present invention provides a vaccine comprising the coronavirus (CoV) vaccine antigen as described herein, or the vector or polynucleotide as described herein, or the protein nanoparticle as described herein, or the virus-like particle as described herein.

In an aspect, the present invention provides a method of inducing an immune response to a coronavirus (CoV) in a subject, the method comprising administering the vaccine as described herein.

In an aspect, the present invention provides a method of enhancing the immune response to coronavirus (CoV) in a subject, the method comprising administering the vaccine as described herein.

In an aspect, the present invention provides a method of preventing or reducing the likelihood of a coronavirus (CoV) infection in a subject, the method comprising administering the vaccine as described herein.

In an aspect, the present invention provides a method of preventing or, reducing the likelihood or severity of a symptom of a coronavirus (CoV) infection in a subject, the method comprising administering the vaccine as described herein.

In an aspect, the present invention provides a method of reducing the severity and/or duration of a coronavirus (CoV) infection in a subject, the method comprising administering the vaccine as described herein.

In an aspect, the present invention provides a method of preventing or reducing viral shedding in a human individual infected with a coronavirus (CoV), the method comprising administering the vaccine as described herein.

In an aspect, the present invention provides a CoV vaccine antigen as described herein or vaccine as described herein for use in one or more of: i) preventing or reducing the likelihood of a CoV infection in a subject; ii) preventing or reducing the likelihood of severity of a CoV symptom in a subject, iii) reducing the severity and/or duration of a CoV infection in a subject; iv) preventing or reducing viral shedding in a subject; and v) treating a CoV infection in a subject.

In an aspect, the present invention provides a kit, device, surface or strip comprising the coronavirus (CoV) vaccine antigen as described herein.

In an aspect, the present invention provides use of the coronavirus (CoV) vaccine antigen in the manufacture of a medicament for one or more of: i) preventing or reducing the likelihood of a CoV infection in a subject; ii) preventing or reducing the likelihood of severity of a CoV symptom in a subject, iii) reducing the severity and/or duration of a CoV infection in a subject; iv) preventing or reducing viral shedding in a subject; and v) treating a CoV infection in a subject.

In an aspect, the present invention provides use of the antigen or encoding sequence in the manufacture of a preparation for treating, preventing or testing relative to coronavirus (CoV) infections in a population. In one embodiment, the stable trimeric S protein as described herein is used to prepare neutralising antibodies.

In an aspect, the present invention provides a soluble S protein trimer, lacking a heterologous trimerization sequence, wherein the S protein trimer is modified to comprise a structural modification which reduces the size of the alanine cavity in the coiled-coil region of the S protein trimer and wherein the S protein trimer elicits neutralising antibody responses.

In an aspect, the description provides a coronavirus S protein vaccine antigen trimer characterised by one or two or more of the following features:
(i) forms as non-disulfide linked trimers;
(ii) forms as stable non-disulfide linked trimers;
(iii) forms trimers that are thermally stable at about 58° C.;
(iv) forms trimers that are more resistant than control (e.g., S2P-FHA) to disruption by treatment with 0.8% w/v SDS with and without B2ME at 25° C.
(v) forms trimers that are more resistant than an appropriate control (e.g., S2P-FHA) to disruption by boiling with 0.8% w/v SDS without B2ME; and
(vi) forms stable soluble trimers without a heterologous trimerization module.

In one embodiment, it is contemplated that the herein described modified coronavirus S antigen displays reduced off-target reactivity because the structural changes induced by the modified coiled-coil region enhance trimer stability and obviate the use of a heterologous trimerization domain to stabilise expressed trimers.

In an aspect, the description provides a polynucleotide comprising a sequence of nucleotides encoding and capable of expression on a recipient cell, a coronavirus S protein antigen modified at least as described herein. In one embodiment, the modified antigen displays enhanced exposure of broadly neutralizing epitopes in an isolate dependent manner compared to an appropriate control.

### BRIEF DESCRIPTION OF THE FIGURES

FIG. 1A) Represents the three-dimensional structure of the SARS-CoV-2 S ectodomain drawn with the coordinates PDB ID 6VSB (Walls et al., 2020). RBM, ACE2 receptor binding motif with receptor-interacting amino acids shown as spheres in grey at the top of the structure; RBD, receptor binding domain in black at the top of the structure with beta strands shown as thick arrows; NTD, N-terminal domain in grey with beta-sheets represented as thick arrows. Central coiled-coil of S2 is shown as 3 vertical helices in dark grey with Ala cavity highlighted in light grey. B) heptad repeat motifs within the coiled-coil sequences of 3 betacoronaviruses. C) a comparison of the central coiled-coil in the prefusion (PDB ID 6VSB (Walls et al., 2020) and postfusion (PDB 6XRA (Cai et al., 2020) conformations. HR1 helices, shown in white in the right-hand side structure form the base of an extension of the coiled-coil that projects upward toward the cellular membrane in the postfusion conformation (Cai et al., 2020). D) Illustration of how the Ala cavity of prefusion S might be filled following substitution with hydrophobic amino acids.

FIG. 2 Illustrates the expression, binding properties and stability of S2P-FHA. A) Superose 6 size exclusion chromatography (SEC) of purified S2P-FHA trimers. B) SDS-PAGE under reducing conditions of purified S2P-FHA. C) Binding of avidin-captured biotinylated S2P-FHA trimers to ACE2-Fc and various human monoclonal antibodies in ELISA. D) Differential scanning fluorimetry of purified S2-FHA proteins performed using SYPRO Orange. The rate of change of fluorescence over time [−d(RFU)/dt] as a function of temperature is shown.

FIG. 3 Shows the expression and stability screening of Ala cavity mutants. A) SDS-PAGE under reducing conditions of S2P-FHA mutants purified from culture supernatants with TALON divalent cation affinity resin. The 10% polyacrylamide gel was stained with Coomassie blue. B) Differential scanning fluorimetry of purified S proteins performed using SYPRO Orange. The rate of change of fluorescence over time [−d(RFU)/dt] as a function of temperature is shown.

FIG. 4 Shows the purification and characterization of selected Ala cavity mutants. A) Superose 6 SEC of S2P-FHA and selected Ala cavity mutants following elution from TALON resin. The calibration standards are thyroglobulin (669 kDa), ferritin, (440 kDa) and IgG (150 kDa). B) Superose 6 SEC of purified trimers following a freeze (−80° C.)-thaw cycle. C) Differential scanning fluorimetry of purified S proteins performed using SYPRO Orange. The rate of change of fluorescence over time [−d(RFU)/dt] as a function of temperature is shown. Representative of at least two independent experiments. D) SDS-PAGE under nonreducing (top) and reducing (bottom) conditions of purified S2P-FHA variant trimers. The 10% polyacrylamide gel was stained with Coomassie blue.

FIG. 5 Shows biolayer interferometry measurements of ACE2-Fc and human monoclonal anti-S 2 IgGs binding to Ala cavity mutants. A) Binding of S2P-FHA, 1016L and 1016/20VI analytes to S ligands immobilized on anti-human IgG capture biosensors. Association was for 300 sec followed by dissociation for 300 sec. S2P=S2P-FHA. B) Relative binding of 300 nM S2P-FHA analytes to various ligands after 240 sec of association shown as a heatmap.

FIG. 6 Shows immunogenicity of Ala cavity mutants. A) Immunization protocol. B) ELISA titres of final-bleed vaccinal sera to plate bound RBD, S1 monomers and S2P-FHA trimers. The immunogen groups are indicated below the graphs. The endpoint was determined as 10-times background OD obtained in the absence of primary antibody. The bars are the geometric means. S2P=S2P-FHA C) Pseudovirus neutralization ID₅₀s of week-16 vaccinal sera. The S genotypes used in S-HIV pseudotype assays are shown below the graphs. A Wilcoxon matched pairs test was used to determine whether the differences in ID₅₀s observed between groups was significant: ns, not significant; *, P<0.05; **, P<0.01; **, P<0.01. The geometric mean neutralization ID₅₀s and fold reduction in mean neutralization of B.1.351 versus Hu-1 pseudotypes are shown below the graph. S2P=S2P-FHA. D) Binding titer of sera to Hu-1 RBD and RBD-NKY (N417N/E484K/N501Y) mutant. The endpoint was determined as 10-times background OD obtained in the absence of primary antibody. The bars are the geometric means. ns: not significant; Kruskal Wallis test. S2P=S2P-FHA.

FIG. 7 Shows Ancestral Hu-1 S-pseudovirus neutralization ID₅₀s of vaccinal sera. ns: not significant; **, P<0.01, Kruskal-Wallis test.

FIG. 8 Shows Pseudovirus neutralization ID₅₀s of week-16 vaccinal sera for mutant S pseudotypes. The S genotypes used in the S-HIV pseudotype assays are shown below the graphs. A Friedman test was used to determine whether the differences in ID₅₀s observed between groups was significant: ns, not significant; *, P<0.05; **, P<0.01; ***, P<0.001; ****, P<0.0001. Hu-1=Ancestral Hu-1.

FIG. 9 Specificity of elicited antibodies assessed by competition ELISA. A) Competition ID50s of vaccinal sera. 0.5 log₁₀ serially diluted final-bleed vaccinal sera were mixed with constant amounts of ACE2-Fc and human monoclonal anti-S IgGs prior to incubation with avidin captured biotinylated S2P-FHA and Ala cavity mutants. The immunogen groups are indicated below the graphs. A) Kruskall-Wallis test was used to determine whether the differences in ID₅₀s observed between groups was significant: ns, not significant; **, P<0.01. B) location of antibody epitopes in the S2P-FHA trimer. S2P=S2P-FHA. B) location of ACE2-Fc and human monoclonal anti-S IgG binding sites (epitopes) in the S trimer.

FIG. 10 Shows the amino acid sequence of SEQ ID NO: 1.

FIG. 11 Shows the amino acid sequences of S2P (SEQ ID NO:2) and S2P-FHA (SEQ ID NO:3).

FIG. 12 Shows the nucleotide sequence of S2P (SEQ ID NO:4)

FIG. 13 Shows the nucleotide sequence of S2P-FHA (SEQ ID NO:5).

FIG. 14 Shows the subject mutant S antigen elicits a stronger immune response against the wild type (Hu-1) virus and a stronger immune response against a highly resistant variant (beta) than current conventional vaccines encoding the wild type virus. Pseudovirus neutralization ID 50 s of week-16 vaccinal sera for mutant S pseudotypes versus vaccine responses generated in humans using conventional vaccines. The S genotypes used in the S-HIV pseudotype assays are indicated below the graphs. Data shows neutralizing antibody responses against the matched WT (Hu-1) virus and the highly resistant variant of concern (Beta Variant) from guinea pigs vaccinated with S2P-FHA protein trimers containing the 1016/20VI (Burnet VI spike on the left) compared to serum obtained 3-5 weeks after two doses of a conventional vaccine in humans (illustrative conventional vaccine (two doses of mRNA or adenovirus vaccines given to humans) on the right. A Wilcoxon matched pairs test was used to determine whether the differences in ID 50 s observed between groups was significant: ns not significant, p≥0.05; **** P<0.0001.

FIG. 15 Shows ELISA binding titers of vaccinal sera. A) ELISA binding titers of vaccinal sera against biotinylated RBD monomers corresponding to Hu-1, Delta and Omicron BA.1 variants (indicated on the x axis) captured on avidin-coated ELISA plates. The immunogen groups are indicated below the variant names. The endpoint was determined as 5-times the background OD obtained in the absence of primary antibody. Horizontal bars are the geometric mean binding titres for each immunogen group. B) ELISA binding titers of vaccinal sera against S2P-FHA corresponding to Hu-1, Delta and Omicron BA.1 variants (indicated on the x axis) bound to ELISA plates. The immunogen groups are indicated below the variant names. The endpoint was determined as 5-times the background OD obtained in the absence of primary antibody. Horizontal bars are the geometric mean binding titres for each immunogen group. *, P<0.05 versus Hu-1 glycoprotein determined in a Kruskal-Wallis test. Where a P value is not indicated, the differences in titre are not statistically significant.

FIG. 16 Shows the results of neutralization assays. A) Pseudovirus neutralization ID50s (ID50=reciprocal dilution of serum causing 50% neutralization) of vaccinal sera. The S variant genotypes used in S-HIV pseudotype assays are shown below the x axis. The immunogen groups from which the sera were obtained are indicated above the graphs. ID50 neutralization titers obtained with sera from individual animals are indicated with various symbols. The bars are the geometric mean ID50s for each immunogen group. A Kruskal-Wallis test was used to determine whether the differences in ID50s observed between variants was significant: ns, not significant. B) Neutralisation assays performed in high-throughput format with HAT-24 cells using Hu-1, Delta, Omicron BA.1 and Beta bona fide infectious SARS-CoV-2 viruses in a microneutralization assay. The SARS-CoV-2 variants used are shown below the x axis. The immunogen groups from which the sera were obtained are indicated above the graphs. ID50 neutralization titers obtained with sera from individual animals are indicated with various symbols. Horizontal bars are the geometric mean ID50s for each immunogen group. A Kruskal-Wallis test was used to determine whether the differences in ID50s observed between variants was significant: ns, not significant; *, P<0.05; **, P<0.01; ***, P<0.001; ****, P<0.0001.

FIG. 17 Shows the geometric mean of biotin-RBD and S2P-FHA ELISA binding titres, neutralization ID50s and mean fold reductions in mean titres for glycoprotein and viral variants relative to Hu-1 from data presented in FIGS. 15 and 16.

FIG. 18 Shows that the introduction of the 1016/20VI (VI) mutation into S2P-FHA derived from the SARS-CoV-2 omicron BA.1 variant of concern (here referred to as S2P.omicron-FHA) confers hyperstability to the glycoprotein trimer. A) Superose 6 SEC profiles of S2P.omicron-FHA, S2P.omicron.VI-FHA expressed in Expi-293F cells and S2P-FHA (derived from Hu-1) expressed in 293FS cells following purification by TALON affinity chromatography. The elution positions of the molecular weight markers, thyroglobulin (669 kDa), ferritin (440 kDa) and aldolase (158 kDa) are indicated above the graphs with arrows. The dashed boxes indicate the fractions that were pooled to obtain the pure trimers shown in B. B) Superose 6 SEC profiles of presumed S2P.omicron-FHA, S2P.omicron.VI-FHA and S2P-FHA trimers obtained from the experiments shown in A. The dashed boxes in A indicate the fractions that were concentrated and subjected to a freeze (−80° C.)-thaw cycle prior to re-elution in the Superose 6 column to obtain B. The elution positions of the molecular weight markers, thyroglobulin (669 kDa), ferritin (440 kDa) and aldolase (158 kDa) are indicated above the graphs with arrows. C) Differential scanning fluorimetry of purified S2P-FHA proteins performed using SYPRO Orange for determining the temperature of thermal unfolding. Representative of at least two independent experiments. D) SDS-PAGE of S2P.omicron-FHA and S2P.omicron. VI-FHA trimers under non-reducing and reducing (1% v/v betamercaptoethanol). In this experiment, the samples were not boiled prior to electrophoresis. m, markers. E) SDS-PAGE of S2P.omicron-FHA and S2P.omicron. VI-FHA trimers under non-reducing and reducing (1% v/v betamercaptoethanol) conditions. The samples were boiled for 5 min prior to electrophoresis. The position of the S2P.omicron.VI-FHA major band under the various conditions is indicated with an arrow. m, markers. F) SDS-PAGE of S2P.omicron-FHA, S2P.omicron.VI-FHA and S2P-FHA trimers under non-reducing (gel on left) and reducing (1% v/v betamercaptoethanol) (gel on right) conditions. Sample buffer containing SDS was added to the samples with and without 1% betamercaptoethanol and the samples were either left at room temperature (25° C.) or boiled (100° C.) for 3 min prior to electrophoresis. Thyroglobulin that had been chemically crosslinked with 1 mM bis(sulfosuccinimidyl)suberate was included to mark the expected position (669 kDa) of the S2P.omicron. VI-FHA trimer. The presumed S2P.omicron.VI-FHA trimer band is indicated with an arrow. Note that S2P.omicron. VI-FHA is disrupted to its monomeric molecular weight only following boiling in the presence of betamercaptoethanol and SDS.

FIG. 19 Shows the binding of human neutralizing monoclonal antibodies to streptavidin-captured biotinylated S2P.omicron-FHA (panels left) and biotinylated S2P.omicron.VI-FHA (panels right) trimers in ELISA. The specificity of the monoclonals is indicated adjacent to the legend.

FIG. 20 Shows that the 1016/20VI (VI) mutation enables trimerization of the prefusion stabilized S ectodomain (residues 16-1208) in the absence of the T4 foldon trimerization tag. A) Cryo-EM structure of the prefusion S trimer ectodomain obtained from solubilised full-length S (Cai et al. 2020). Receptor binding domains are shown in black, the central coiled-coil of S2 in grey with the alanine cavity (in which the 1016/20VI mutation was introduced) is highlighted in dark grey. The 3 helices at the bottom of the trimer are the part of the stem that is visible in cryo-EM structures. The dashed line rectangle shows the portion of the stem that is not visible in cryo-EM structures. The crystal structure of the foldon domain solved in isolation (Guthe et al 2004) has been added for illustrative purposes. The structure of residues 1217-1237 of the S2 transmembrane domain (TMD) in lipid bicelles solved by nuclear magnetic resonance (Fu and Chou, 2021) is also shown for illustrative purposes. The vertical lines indicate the extent of the construct s analysed by SEC, thermofluor assay and SDS-PAGE in panels B-D, respectively. B) Size exclusion chromatography of S2P-FHA, S2P-1208.H6 and S2P.VI-1208.H6. First panel: Superose 6 elution profile of S2P-FHA purified from 293FS cells by TALON affinity chromatography. The arrows show the elution positions of molecular weight standards: thyroglobulin (669 kDa), ferritin (440 kDa) and aldolase (158 kDa). Second panel: Superose 6 elution profile of S2P-1208.H6 (lacking the foldon domain) purified from 293FS cells by TALON affinity chromatography. Third panel: Superose 6 elution profile of S2P.VI-1208.H6 (containing the 1016/20VI mutation and lacking the foldon domain) purified from 293FS cells by TALON affinity chromatography. Fourth panel: Superose 6 elution profile of S2P.VI-1208.H6 (containing the 1016/20VI mutation and lacking the foldon domain) purified from Expi293F cells by hiTRAP affinity chromatography. C) Differential scanning fluorimetry of purified S trimers following a freeze (−80° C.)-thaw cycle performed using SYPRO Orange for determining the temperature of thermal unfolding. Representative of at least two independent experiments. D) SDS-PAGE of purified proteins under non-reducing and reducing conditions. m, markers.

FIG. 21A) Shows the Schematic of S2P-1273, S2P-FHA and S2P-1208.H6 constructs. L, native leader peptide, NTD, N-terminal domain of S1, RBD receptor binding domain, (P) GSAS mutation ablating furin site, tPAL, tissue plasminogen activator leader peptide; TMD, transmembrane domain, FHA, foldon-His8-avitag sequence. B) Shows a SDS-PAGE-western blot of Hu-1, Delta and Omicron BA.1 S2P-1273 and S2P.VI-1273 glycoproteins expressed in 293T cells. The S proteins were detected with rabbit-anti-S1 polyclonal antibody. The gel on the left is a 10% polyacrylamide gel. The gel on the right is a 4-12% polyacrylamide gradient gel.

FIG. 22 Shows binding of ACE2-Fc and human monoclonal NAbs to S2P-1273 glycoproteins expressed on the surface of transfected 293T cells as determined by FACS. The transfected cells were gently detached from culture plates and the intact cells were stained with ACE2-Fc and various human monoclonal NAbs and AlexaFluor-conjugated anti-human immunoglobulin. The cells were counterstained with propidium iodide to enable the exclusion of dead cells from analyses. HC33.1 is a HCV-specific NAb and is used as an isotype (IgG1) control.

FIG. 23A) Gating of unstained (nil), moderately (lo) fluorescent and highly (hi) fluorescent S2P-1273 expressing cells. B) Representative data showing distributions of S2P-1273-expressing cells stained with ACE2-Fc and 5 NAbs directed to various epitopes in S within nil, lo and hi gates in graphical form.

FIG. 24 Amino acid sequence of S2P.omicron-FHA (SEQ ID NO:6).

FIG. 25 DNA sequence of S2P.omicron-FHA (SEQ ID NO:7).

FIG. 26 Amino acid sequence of S2P-1208.H6 (SEQ ID NO:8).

FIG. 27 DNA sequence of S2P-1208.H6 (SEQ ID NO:9).

FIG. 28 Protein sequence of S2P-1273 (SEQ ID NO:10).

FIG. 29 DNA sequence of S2P-1273 (SEQ ID NO:11).

FIG. 30 Protein sequence of S2P.delta-1273 (SEQ ID NO:12).

FIG. 31 DNA sequence of S2P.delta-1273 (SEQ ID NO:13),

FIG. 32 Protein sequence of S2P.omicron-1273 (SEQ ID NO:14).

FIG. 33 DNA sequence of S2P.omicron-1273 (SEQ ID NO:15).

FIG. 34 Example of an mRNA vaccine sequence encoding the S2P-1208 open reading frame (SEQ ID NO:16). “U” in this sequence indicates pseudouridine or 1-methylpseudouridine.

FIG. 35 Example of an mRNA vaccine sequence encoding the S2P.VI-1273 open reading frame (SEQ ID NO:17). “U” in this sequence indicates pseudouridine or 1-methylpseudouridine.

FIG. 36 Example of an mRNA vaccine sequence encoding the S2P.delta.VI-1273 open reading frame (SEQ ID NO:18). “U” in this sequence indicates pseudouridine or 1-methylpseudouridine.

FIG. 37 Example of an mRNA vaccine sequence encoding the S2P.omicron.VI-1273 open reading frame (SEQ ID NO:19). “U” in this sequence indicates pseudouridine or 1-methylpseudouridine.

FIG. 38 Shows SDS-PAGE/western blot analysis of S2P-1273 and S2P.omicron-1273 glycoproteins derived from transfected 293T cell lysates following various heat treatments in the presence of 1.2% (w/v) SDS and 0.25% (v/v) betamercaptoethanol. AA: constructs containing Ala at amino acid positions 1016 and 1020; VI: constructs containing Val and Ile at amino acid positions 1016 and 1020, respectively. AA and VI versions of purified S2P.omicron-FHA trimers treated with 0.67% SDS for 5 min at room temperature were included to indicate the positions of S monomers and trimers, respectively. The samples were electrophoresed on 5% SDS-PAGE gels, transferred to nitrocellulose and blotted with rabbit anti-S1 and anti-rabbit IRDye 800. The blots were scanned in a LiCOR Odyssey instrument.

FIG. 39 Shows that the 1016/20VI (VI) mutation stabilizes the trimerization of the prefusion stabilized omicron BA.1 S ectodomain (residues 16-1208) in the absence of the T4 foldon trimerization tag. A) Superose 6 size exclusion chromatography of S2P.omicron.BA.1-1208.H6 (top) and S2P.omicron.BA.1.VI-1208.H6 (bottom) purified from Expi293F cells by hiTRAP affinity chromatography. B) Superose 6 size exclusion chromatography of S2P.omicron.BA.1-1208.H6 (top) and S2P.omicron.BA.1.VI-1208.H6 (top) trimers purified in A following a freeze (−80° C.)-thaw cycle. C) Differential scanning fluorimetry of purified S trimers following a freeze (−80° C.)-thaw cycle performed using SYPRO Orange for determining the temperature of thermal unfolding. D) SDS-PAGE and Coomassie blue staining of purified proteins under non-reducing conditions. WT: Ala at amino positions 1016 and 1020; VI: Val and Ile at amino positions 1016 and 1020, respectively.

FIG. 40 Shows biolayer interferometry measurements of ACE2-Fc and human monoclonal anti-S IgGs binding to Ala cavity mutants. A) Binding of S2P-FHA and S2P.VI-FHA analytes derived from the Hu-1 sequence to S ligands immobilized on anti-human IgG capture biosensors. B) Binding of S2P-FHA and S2P.VI-FHA analytes derived from the omicron.BA.1 sequence to S ligands immobilized on anti-human IgG capture biosensors. Association was for 300 sec followed by dissociation for 300 sec.

FIG. 41 Protein sequence of S2P.omicron-1208 (SEQ ID NO:25).

FIG. 42 Protein sequence of DNA sequence of S2P.omicron-1208 (SEQ ID NO: 26).

### KEY TO SEQUENCE LISTING

| | | |
|---|---|---|
| SEQ ID NO: 1 | Severe acute respiratory syndrome | Amino acid sequence |
| | coronavirus 2 surface glycoprotein | |
| SEQ ID NO: 2 | Severe acute respiratory syndrome | Amino acid sequence |
| | coronavirus 2 S2P | |
| SEQ ID NO: 3 | Severe acute respiratory syndrome | Amino acid sequence |
| | coronavirus 2 S2P-FHA | |
| SEQ ID NO: 4 | Severe acute respiratory syndrome | Nucleotide sequence |
| | coronavirus 2 S2P | |
| SEQ ID NO: 5 | Severe acute respiratory syndrome | Nucleotide sequence |
| | coronavirus 2 S2P-FHA | |
| SEQ ID NO: 6 | Severe acute respiratory syndrome | Amino acid sequence |
| | coronavirus 2 S2P omicron-FHA | |
| SEQ ID NO: 7 | Severe acute respiratory syndrome | Nucleotide sequence |
| | coronavirus 2 S2P omicron-FHA | |
| SEQ ID NO: 8 | Severe acute respiratory syndrome | Amino acid sequence |
| | coronavirus 2 S2P-1208.H6 | |
| SEQ ID NO: 9 | Severe acute respiratory syndrome | Nucleotide sequence |
| | coronavirus 2 S2P-1208.H6 | |
| SEQ ID NO: 10 | Severe acute respiratory syndrome | Amino acid sequence |
| | coronavirus 2 S2P-1273 | |
| SEQ ID NO: 11 | Severe acute respiratory syndrome | Nucleotide sequence |
| | coronavirus 2 S2P-1273 | |
| SEQ ID NO: 12 | Severe acute respiratory syndrome | Amino acid sequence |
| | coronavirus 2 S2P.delta.1273 | |
| SEQ ID NO: 13 | Severe acute respiratory syndrome | Nucleotide sequence |
| | coronavirus 2 S2P.delta.1273 | |
| SEQ ID NO: 14 | Severe acute respiratory syndrome | Amino acid sequence |
| | coronavirus 2 S2P.omicron-1273 | |
| SEQ ID NO: 15 | Severe acute respiratory syndrome | Nucleotide sequence |
| | coronavirus 2 S2P.omicron-1273 | |
| SEQ ID NO: 16 | Severe acute respiratory syndrome | Nucleotide sequence |
| | coronavirus 2 S2P-1208 open reading | |
| | frame | |
| SEQ ID NO: 17 | Severe acute respiratory syndrome | Nucleotide sequence |
| | coronavirus 2 S2P.VI-1273 open reading | |
| | frame | |
| SEQ ID NO: 18 | Severe acute respiratory syndrome | Nucleotide sequence |
| | coronavirus 2 S2P.delta.VI-1273 open | |
| | reading frame | |
| SEQ ID NO: 19 | Severe acute respiratory syndrome | Nucleotide sequence |
| | coronavirus 2 S2P.omicron.VI-1273 open | |
| | reading frame | |
| SEQ ID NO: 20 | Foldon | Amino acid sequence |
| SEQ ID NO: 21 | FHA tag | Amino acid sequence |
| SEQ ID NO: 22 | Linker | Amino acid sequence |
| SEQ ID NO: 23 | Linker | Amino acid sequence |
| SEQ ID NO: 24 | Restriction site downstream sequence | Nucleotide sequence |
| SEQ ID NO: 25 | Amino acid sequence of S2P.omicron- | Amino acid sequence |
| | 1208 | |
| SEQ ID NO: 26 | DNA sequence of S2P.omicron-1208 | Amino acid sequence |

### Discussion of Embodiments

Unless defined otherwise, all technical and scientific terms used herein have the same meanings as commonly understood by one of ordinary skill in the art to which this disclosure belongs. Any materials and methods similar or equivalent to those described herein can be used to practice or test the present disclosure. Practitioners are particularly directed to Ausubel et al., Current Protocols in Molecular Biology, Supplement 47, John Wiley & Sons, New York, 1999; Colowick and Kaplan, eds., Methods In Enzymology, Academic Press, Inc.; Weir and Blackwell, eds., Handbook of Experimental Immunology, Vols. I-IV, Blackwell Scientific Publications, 1986; Kontermann and Dubel (Ed), Antibody Engineering, Vol 1-2, Ed., Springer Press, 2010) for definitions and terms of the art and other methods known to the person skilled in the art.

The sequence of Coronavirus Spike(S) protein from the ancestral Hu-1 strain is described by Wu, F., et al Nature 579 (7798), 265-269 (2020) and in NCBI Reference Sequence: YP_009724390.1. This strain may also be referred to as “wild-type”, “ancestral” and “parental” strain herein.

As used herein “antigen” refers to a substance capable of stimulating an immune response.

As used herein “protection” refers to immunity or partial immunity against a coronavirus infection.

“Alanine cavity” or “cavity” refers herein to the observed region and reduced interaction between monomers of the trimeric structure within the coiled-coil of SARS-CoV S protein due to an absence of amino acids with non-polar side chains bulkier than that of alanine or aromatic residues. In an embodiment, the alanine cavity comprises A1016 and A1020 as set out in any one of SEQ ID NO:1 to SEQ ID NO:3.

As used herein, with reference to illustrative embodiments of the invention, a reference to S2P, unless specifically specified, includes embodiments with and without the FHA sequence.

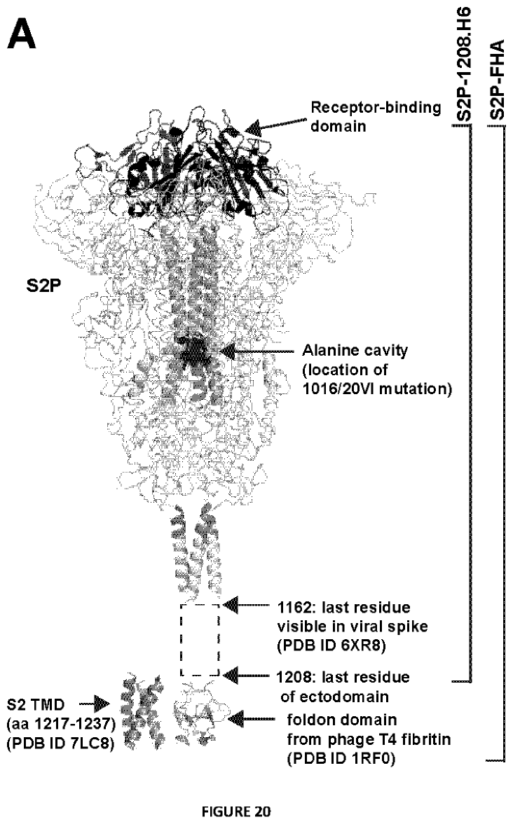

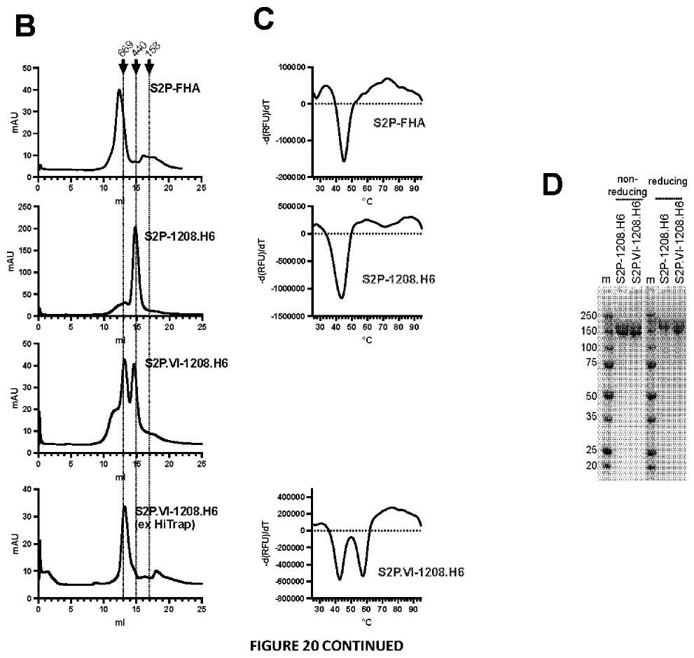

As used herein, “trimerization sequence” refers to a sequence found at the C terminal region of the S-protein monomers which facilitates trimerization of the S protein trimer. In some embodiments, the trimerization domain is a heterologous sequence, not found within coronaviruses. In one embodiment, the trimerization sequence is a heterologous sequence, not found within SARS-COV2. In some embodiments, the trimerization sequence is the trimeric foldon domain of bacteriophage T4 fibritin or a modified version thereof (FIG. 20A). In some embodiments, the trimerization sequence is a coiled-coil, an artificial coiled-coil or a modified coiled-coil. In some embodiments, the trimerization sequences was designed de novo, In some embodiments, the trimerization sequence is a trimeric foldon domain of bacteriophage T4 fibritin or a modified version thereof. In some embodiments, the trimerization sequence is a native CoV trimerization sequence (in some embodiments this is the native transmembrane domain). In some embodiments, the trimerization sequence comprises or consists of residues 1209-1256 of the S protein monomer. In some embodiments, the trimerization sequence comprises or consists of residues 1217-1237 of the S protein monomer.

### Coronavirus

The term “Coronaviridae”, refers to viruses known by the common name of “Coronavirus” or “CoV” which are enveloped, positive sense, single-stranded RNA viruses. There are two subfamilies of Coronaviridae, Letovirinae and Orthocoronavirinae. In one embodiment, the CoV is selected from the genera Alphacoronavirus (alphaCoV), Betacoronavirus (betaCoV), Gammacoronavirus (gammaCoV) and Deltacoronavirus (deltaCoV). In one embodiment, the alphaCoV is selected from coronavirus 229E (HCoV-229E), human coronavirus NL63 (HCoV-NL63), transmissible gastroenteritis virus (TGEV), porcine epidemic diarrhea virus (PEDV), feline infectious peritonitis virus (FIPV) and canine coronavirus (CCoV). In one embodiment, the betaCoV is selected from human coronavirus HKU1 (HCoV-HKU1), Human coronavirus OC43 (HCoV-OC43), Severe acute respiratory syndrome-related coronavirus (SARS-CoV), Severe acute respiratory syndrome-related coronavirus-2 (SARS-CoV-2), Middle-East respiratory syndrome-related coronavirus (MERS-CoV), murine hepatitis virus (MHV) and/or bovine coronavirus (BCoV). In one embodiment, the CoV is capable of infecting a human. In one embodiment, the CoV capable of infecting a human is selected from: SARS-CoV-2, HCoV-OC43, HCoV-HKU1, HCoV-229E, HCoV-NL63, SARS-CoV, and MERS-CoV or a subtype or variant thereof.

In one embodiment, the CoV is SARS-CoV-2 or a subtype or variant thereof. In an embodiment, SARS-CoV-2 is SARS-CoV-2 hCoV-19/Australia/VIC01/2020. In one embodiment, SARS-CoV-2 comprises the sequences as described in NCBI Reference Sequence: NC_045512.2. In one embodiment, SARS-CoV-2 comprises the sequence as described in GenBank: MN908947.3 or a variant thereof. Examples of SARS-CoV-2 variants are described, for example, in Shen et al., 2020, Tang et al., 2020, Phan et al., 2020, Khan et al., 2020, Foster et al., 2020, Vasireddy et al., 2021, Winger et al., 2021 and Sanyaolu et al., 2021.

In one embodiment, the CoV variant is at least 90% identical to the parental sequence. In one embodiment, the variant is at least 92% identical to the parental sequence. In one embodiment, the variant is at least 93% identical to the parental sequence. In one embodiment, the variant is at least 94% identical to the parental sequence. In one embodiment, the variant is at least 95% identical to the parental sequence. In one embodiment, the variant is at least 96% identical to the parental sequence. In one embodiment, the variant is at least 97% identical to the parental sequence. In one embodiment, the variant is at least 98% identical to the parental sequence. In one embodiment, the variant is at least 99% identical to the parental sequence. In an embodiment, the parent strain (also referred to as the ancestral strain) is Hu-1 strain is described by Wu, F., et al Nature 579 (7798), 265-269 (2020). In some embodiments, the parental strain is SARS-CoV-2 hCoV-19/Australia/VIC01/2020. In some embodiment, the parental strain is BetaCoV/Ancestral/WIV04/2019.

In an embodiment, the CoV is a “Variant of Interest” also referred to as a “VOI”. As used herein a VOI is a variant of a coronavirus that is associated with genetic changes that are predicted or known to affect virus characteristics such as transmissibility, disease severity, immune escape, diagnostic or therapeutic escape; and identified to cause significant community transmission or multiple disease clusters (in the case of SARS-CoV-2 COVID19 clusters), in multiple countries with increasing relative prevalence alongside increasing number of cases over time, or other apparent epidemiological impacts to suggest an emerging risk to global public health.

In an embodiment, the CoV is a “Variant of Concern” also referred to as a “VOC”. As used herein a VOC is a variant of a coronavirus that is associated with one or more of the following changes at a degree of global public health significance: increase in transmissibility or detrimental change in epidemiology (in the case of SARS-CoV-2 the detrimental change is in COVID-19 epidemiology); increase in virulence or change in clinical disease presentation; or a decrease in effectiveness of public health and social measures or available diagnostics, vaccines, therapeutics.

In an embodiment, the CoV is a VOC or VOI as described in Vasireddy et al (2021), Winger et al (2021) or Sanyaolu et al (2021). In an embodiment, the CoV is classified as a VOC, VOI or VHC by a health regulatory body e.g. the World Health Organisation (WHO), the United States Center of Disease Control (CDC), the European Centre for Disease Prevention and Control (ECDC) or an equivalent local government health regulatory body in a specific jurisdiction. In an embodiment, the coronavirus is classified as a VOC or VOI by WHO. In an embodiment, the coronavirus is classified as a VOC, VOI or VHC by the CDC. In an embodiment, the coronavirus is classified as a VOC or VOI by ECDC.

In an embodiment, the CoV is a “Variant of High Consequence” also referred to as a “VHC”. In an embodiment the VHC as clear evidence that prevention measures or medical countermeasures have significantly reduced effectiveness relative to previously circulating variants. In addition to the characteristics of a VOC a VHC can have one or more of the following impacts on medical countermeasures: demonstrated failure of diagnostic test targets; evidence to suggest a significantly reduction in vaccine effectiveness, a disproportionately high number of vaccine breakthrough cases, or very low vaccine-induced protection against severe disease; significantly reduced susceptibility to multiple emergency use authorization or approved therapeutics and more severe clinical disease and increased hospitalizations

In an embodiment, where the CoV is SARS-CoV-2 VOC, the VOC comprises one or more of the following mutations: 69del, 70del, 144del, E484K/Q, S494P, N501Y, A570D, D614G, P681H, T7161, S982A, D1118H, K1191N, D80A, D215G, 241del, 242del, 243del, K417N, E484K, N501Y, D614G, A701V, T19R, V70F, T95I, G142D, E156-, F157-, R158G, A222V*, W258L*, K417N/T*, L452R, T478K, D614G, P681H/R, D950N, L18F, T20N, P26S, D138Y, R190S, K417T, E484K, N501Y, D614G, H655Y, T1027I, E484K, H655Y A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K and L981F.

In an embodiment, where the CoV is SARS-CoV-2 VOC, the VOC comprises one or more of the following RBD mutations: K417T/N, N439K, L452R, Y453F, S477N, T478K, E484K/Q, and N501Y. In an embodiment, the VOC comprises one or more of the following NTD mutations: 70del, 156-157del and 242-245del.

In an embodiment, the VOC is B.1.1.7 or a variant thereof. In an embodiment, the VOC is B.1.351 or a variant thereof. In an embodiment, the VOC is B.1.351.2 or a variant thereof. In an embodiment, the VOC is B.1.351.2 or a variant thereof. In an embodiment, the VOC is B.1.351.3 or a variant thereof. In an embodiment, the VOC is P1 or a variant thereof. In an embodiment, the VOC is P1.1 or a variant thereof. In an embodiment, the VOC is P1.2 or a variant thereof. In an embodiment, the VOC is B.1.617.2 or a variant thereof. In an embodiment, the VOC is AY.1 or a variant thereof. In an embodiment, the VOC is AY.2 or a variant thereof. In an embodiment, the VOC is AY.3 or a variant thereof. In an embodiment, the VOC is B.1.1.529 or a variant thereof. In an embodiment, the VOC is BA.1 or a variant thereof. In an embodiment, the VOC is BA.2 or a variant thereof. In an embodiment, the VOC is BA.3 or a variant thereof. In an embodiment, the VOC is BA.4 or a variant thereof. In an embodiment, the VOC is BA.5 or a variant thereof.

In an embodiment, where the CoV is SARS-CoV-2 VOI, the VOI comprises one or more of the following mutations: L452R, D614G, S13I, W152C, A67V, 69del, 70del, 144del, E484K, Q677H, F888L, L5F, D80G, T95I, Y144, F157S, D253G, L452R, S477N, E484K, A701V, T859N, D950H and Q957R, N501Y, P681R, P681H, E484Q, P681R, S477N, L452Q and F490S. In an embodiment, the VOI is B.1.525 or a variant thereof. In an embodiment, the VOI is B.1.526 or a variant thereof. In an embodiment, the VOI is B.1.617.1 or a variant thereof. In an embodiment, the VOI is C37 or a variant thereof. In an embodiment, the VOI is B.1.427 or a variant thereof. In an embodiment, the VOI is B.1.429 or a variant thereof. In an embodiment, the VOI is P2 or a variant thereof. In an embodiment, the VOI is B.1.525 or a variant thereof. In an embodiment, the VOI is P3 or a variant thereof. In an embodiment, the VOI is B.1.620 or a variant thereof. In an embodiment, the VOI is B.1.621 or a variant thereof. In an embodiment, the VOI is C.37 or a variant thereof.

CoV infections cause can cause respiratory, enteric, hepatic, and neurological diseases in different animal species, including camels, cattle, cats, and bats. CoV can be transmitted from one individual to another through contact of viral droplets with mucosa. Typically, viral droplets are airborne and inhaled via the respiratory tract including the nasal airway. Typically, the individual is a human individual. In some embodiments, the individual is a live stock or domestic animal. Typically, during an infection, CoV can be found in the upper respiratory tract, for example the nasal passages. In some examples, CoV can be found in the lower respiratory tract, for example the bronchi and/or alveoli.

In an embodiment, a CoV infection causes one or more symptoms selected from one or more of: fever, cough, sore throat, shortness of breath, viral shedding respiratory insufficiency, runny nose, nasal congestion, malaise, bronchitis, headache, muscle pain, dyspnea, moderate pneumonia, severe pneumonia, acute respiratory distress syndrome (ARDS). In an embodiment, the ARDS is selected from mild ARDS (defined as 200 mmHg<PaO2/FiO2≤300 mmHg), moderate ARDS (defined as 100 mmHg<PaO2/FiO2≤200 mmHg) and severe ARDS (defined as PaO2/FiO2≤100 mmHg). In an embodiment, a SARS-CoV-2 infection can cause one or more symptoms selected from one or more of: fever, cough, sore throat, shortness of breath, viral shedding, respiratory insufficiency, runny nose, nasal congestion, malaise, bronchitis, headache, muscle pain, dyspnea, moderate pneumonia, severe pneumonia, acute respiratory distress syndrome (ARDS). In an embodiment, the CoV infection is asymptomatic.

### SARS-CoV-2

In an embodiment, the coronavirus vaccine antigen as described herein, elicits an immune response to SARS-CoV-2. In an embodiment, the coronavirus vaccine antigen as described here comprises a SARS-CoV-2 S protein trimer.

SARS-CoV-2 has four major structural proteins: spike(S), membrane (M) and envelope (E) proteins, and nucleocapsid (N) protein. S, M and E are embedded in the viral surface envelope and N is located in in the ribonucleoprotein. The S protein recognizes the host cellular receptor to initiate virus entry.

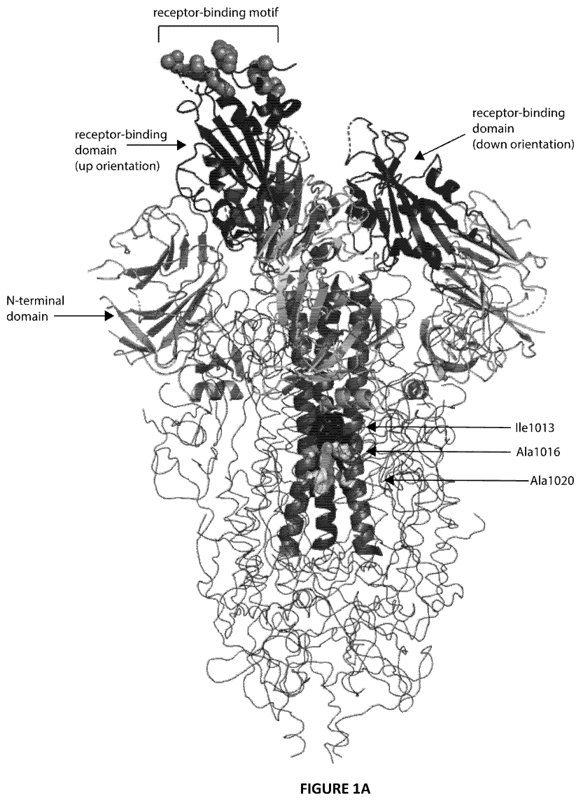

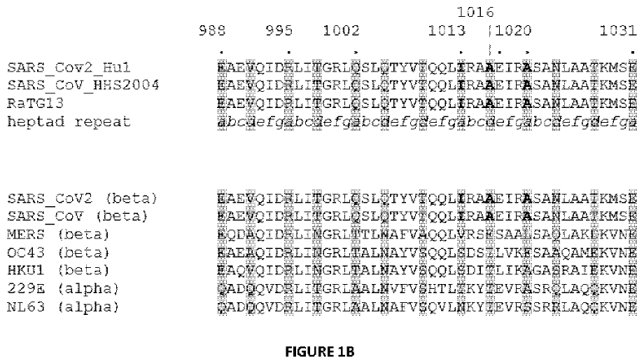

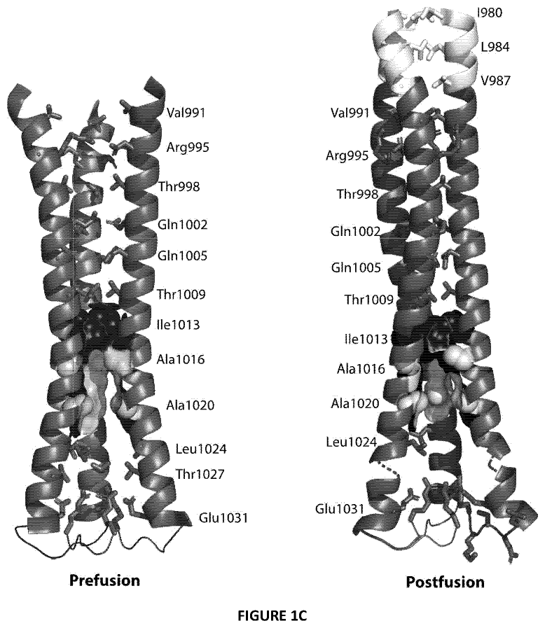

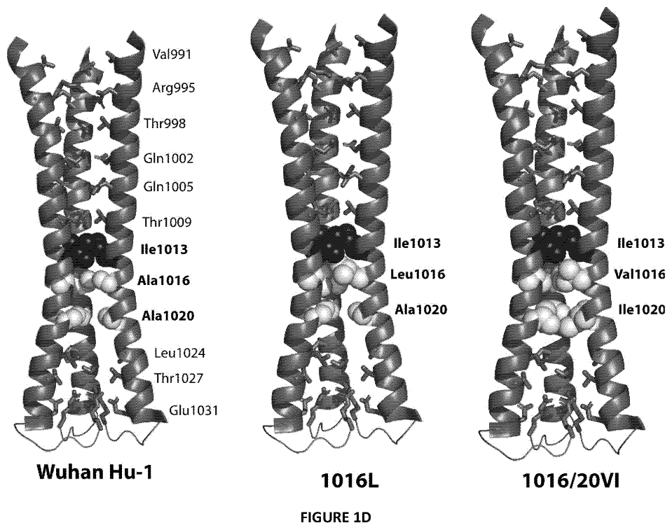

The viral S glycoprotein mediates receptor attachment and virus-cell membrane fusion and is the target of NAbs (Duan et al., 2020; Finkelstein et al., 2021; Walls et al., 2020; Hoffmann et al., 2020). The mature spike comprises 2 functional subunits, S1 and S2, that are derived from a polyprotein precursor, S, by furin cleavage of an oligobasic motif as it transits the Golgi. ACE2 receptor attachment is mediated by the RBD within the large subunit, S1, while membrane fusion is mediated by the small subunit, S2, which contains the fusion peptide. S1 and S2 form a heterodimer via non-covalent interactions; a coiled-coil-forming α-helix of S2 (amino acids 986-1033; referred to as CH) forms the core of the trimer (Wrapp et al., 2020) (FIG. 1A). A membrane-spanning sequence at the C-terminus of S2 stabilizes the trimer and anchors it to the viral or cell membrane (Fu et al., 2021). The ACE2 RBD sits atop the S1 glycoprotein trimer and presents in “up” ACE2-binding-ready and “down” inert orientations (Ke et al., 2020). Following receptor attachment, S2 is cleaved by the TMPRSS2 protease at the cell surface to liberate the fusion peptide and full fusion activation. The S glycoprotein mediates membrane fusion via a class I mechanism whereby an activation trigger (ACE2-binding by S1, TMPRSS2 cleavage of S2) causes the S2 subunit of the metastable pre-fusion trimer to refold into a stable trimer of hairpins, bringing the N-terminal fusion peptide and C-terminal membrane spanning sequences together such that their associated membranes fuse (Cai et al., 2020).

Class I viral fusion glycoproteins such as S of betacoronaviruses, Env of retroviruses, HA of orthomyxoviruses contain a central coiled-coil, which act as a scaffold for the conformational changes required for the membrane fusion process (Bullough et al., 1994; Cai et al., 2020; Chan et al., 1997; Julien et al., 2013; Walls et al., 2017; Walls et al., 2020; Weissenhorn et al., 1997; Wilson et al., 1981; Wrapp et al., 2020) (FIG. 1A). In the case of SARS-CoV-2, prefusion, the coiled-coil comprises amino acids amino acids 988 to 1031 set out in SEQ ID NO:1 (NCBI reference sequence YP_009724390.1). This region for examples SARS-CoV-2 strains is shown in FIG. 1B. Post-fusion the coiled-coil sequence is extended comprising amino acids 913 to 1031 set out in SEQ ID NO:1 (NCBI reference sequence YP_009724390.1). In some embodiments, the residues 986 and 987 are modified to prolines (K986P; V987P).

The inward-facing positions of a coiled-coil are usually occupied by hydrophobic residues in a 3-4 repeat. In the cases of SARS-CoV and SARS-CoV-2 S, these positions are mostly occupied by polar residues that mediate few inter-helical contacts in the prefusion trimer (FIG. 1 B). In the postfusion trimer, the N-terminal 2/3 of the coiled-coil is brought together by the packing of HR1 helices that extends the coiled-coil in an N-terminal direction by 110 Å. In this conformation the inward facing residues are close enough for hydrogen bonds to form (FIG. 1 C). Ile1013 forms a small hydrophobic core through inter-helical contacts with 11013 and with L1012. These interactions form a hydrophobic ceiling above a cavity formed by A1016 and A1020 that occupy central positions of the coiled-coil (FIG. 1A-D).

### Vaccine Antigen

In an aspect, the present invention provides a coronavirus vaccine antigen comprising a CoV S protein trimer wherein the S protein trimer is modified to comprise a structural modification which reduces the size of the alanine cavity in the coiled-coil of the S protein trimer and wherein the S protein trimer elicits neutralising antibody responses.

In an aspect, the structural modification stabilises the S protein trimer. As used herein “stabilised” refers to increasing one or more of the: thermal stability, longevity, immunogenicity and production stability, yield or homogeneity of the S protein trimer, and denaturation stability. In an embodiment, the stability is increased in vitro and/or in vivo stability. In an embodiment, the in vivo stability is increased (when administered to a subject or upon assembly in a subject e.g. after translation from a nucleic acid such as an mRNA vaccine). In an embodiment, the stability is increased in vitro (e.g. during production processes).

In an embodiment, the structural modification stabilises the S protein trimer by reducing the size of the alanine cavity in the coiled-coil. In an embodiment, the alanine cavity is partially filled or completely filled. In an embodiment, the alanine cavity is conformationally altered. In an embodiment, the size of the alanine cavity is reduced by at least 5%, or at least 10%, or at least 20%, or at least 30%, or at least, 40%, or at least 50%, or at least 60%, or at least 70% or at least 80%, or at least 90%, or 100%. In an embodiment, the size of the alanine cavity is reduced by at least 5%. In an embodiment, the size of the alanine cavity is reduced by at least 10%. In an embodiment, the size of the alanine cavity is reduced by at least 20%. In an embodiment, the size of the alanine cavity is reduced by at least 30%. In an embodiment, the size of the alanine cavity is reduced by at least 40%. In an embodiment, the size of the alanine cavity is reduced by at least 50%. In an embodiment, the size of the alanine cavity is reduced by at least 60%. In an embodiment, the size of the alanine cavity is reduced by at least 70%. In an embodiment, the size of the alanine cavity is reduced by at least 80%. In an embodiment, the size of the alanine cavity is reduced by at least 90%. In an embodiment, the size of the alanine cavity is reduced by 100%.

In an embodiment, the size of the alanine cavity is reduced by about 10% to 100%, or about 10% to about 90%, or about 20% to about 90%, or about 20% to about 80%, or about 30% to about 80%, or about 40% to about 80%, or about 50% to about 80%. In an embodiment, the size of the alanine cavity is reduced by about 10% to 100%. In an embodiment, the size of the alanine cavity is reduced by about 10% to 90%. In an embodiment, the size of the alanine cavity is reduced by about 20% to 90%. In an embodiment, the size of the alanine cavity is reduced by about 20% to 80%. In an embodiment, the size of the alanine cavity is reduced by about 30% to 80%. In an embodiment, the size of the alanine cavity is reduced by about 40% to 80%. In an embodiment, the size of the alanine cavity is reduced by about 50% to 80%.

In an embodiment, the structural modification increases the stability of the S protein trimer compared to the S protein trimer lacking the structural modification.

In an embodiment, the structural modification increases the temperature at which the S protein trimer degrades compared to the S protein trimer lacking the structural modification. In an embodiment, degrades or degradation refers to exposure of the hydrophobic residues at the core of the S protein trimer.

In an embodiment, the structural modification increases the melting temperature of the S protein trimer compared to the S protein trimer lacking the structural modification. In an embodiment, the structural modification increases the thermal stability of the S protein trimer. In an embodiment, the structural modification increases the melting temperature of the S protein trimer by about 5° C. to about 25° C. In an embodiment, the structural modification increases the melting temperature of the S protein trimer by about 5° C. to about 23° C. In an embodiment, the structural modification increases the melting temperature of the S protein trimer by about 5° C. to about 23° C. In an embodiment, the structural modification increases the melting temperature of the S protein trimer by about 10° C. to about 23° C. In an embodiment, the structural modification increases the melting temperature of the S protein trimer by about 10° C. to about 20° C. In an embodiment, the structural modification increases the melting temperature of the S protein trimer by about 5° C. to about 15° C. In an embodiment, the structural modification increases the melting temperature of the S protein trimer by about 5° C. to about 10° C.

In an embodiment, the structural modification increase the stability of the S protein trimer to denaturing conditions. Denaturing conditions include for example boiling in in the presence of detergent (e.g. sodium dodecyl sulfate) or treating with detergent (e.g. sodium dodecyl sulfate) with and without 2 betamercaptoethanol at room temperature.

In one embodiment, the present specification enables a method of improving the stability and/or expression of coronavirus S antigen.

In an embodiment, the CoV vaccine antigen is soluble. In an embodiment, the CoV vaccine antigen as described herein does not comprise the FHA sequence. In an embodiment the CoV vaccine antigen is stabilised in a pre-fusion S protein trimer confirmation. In an embodiment, the ACE2 receptor binding domain (RBD) of the S protein trimer is in a down (non-ACE2-binding ready) orientation. In an embodiment, when in the RBD-down orientation neutralising antibodies are generated that recognise the S-trimer in an RBD-down conformation in addition to RBD-up directed neutralising antibodies.

In an embodiment, the CoV vaccine antigen lacks a trimerization sequence. In an embodiment, the CoV vaccine antigen lacks a transmembrane domain. In an embodiment, the CoV vaccine antigen lacks a foldon sequence/domain.

In an embodiment, when the RBD is in the down orientation other non-RBD epitopes are in favourable positions for generating additional non-RBD neutralising antibodies.

In an embodiment, the structural modification is in the coiled-coil region. In an embodiment, the structural modification stabilises the coiled-coil region.

In an embodiment, the structural modification in the coiled-coil in S2 has an allosteric effect on the immunogenicity of S1 that enhances the immune response against CoV variants as described herein.

In an embodiment, the CoV vaccine antigen is suitable for intra-dermal administration. In an embodiment, the CoV vaccine antigen is suitable for oral administration. In an embodiment, the CoV vaccine antigen is suitable for pulmonary administration. In an embodiment, the CoV vaccine antigen is suitable for nasal administration.

### S Protein Monomers of the Coronavirus Vaccine Antigen

In an embodiment, S protein monomer in the S-protein trimer can be an ancestral SARS-CoV-2 sequences as described herein (e.g. NCBI Reference Sequence: YP_009724390.1) or can be a more recent variant such as a VOC, VOI or a VHC as described herein (e.g. delta, beta, omicron). In an embodiment, the S protein monomer is an ancestral SARS-CoV-2 sequence modified to comprise one or more mutations present in a VOC, VO1 or a VHC as described herein. In an embodiment, the modification is selected from one or S13I, L18F, T19R, T20N, P26S, A67V, delH69-V70, D80A, T95I, D138Y, G142D, delY144, W152C, E154K, E156del, F157del, R158G, R190S, D215G, del242-245, D253G, R246I, K417N/T, N439K, L452R/Q, Y453F, S477N, T478K, E484K/Q, N501Y, F565L, A570D, D614G, H655Y, Q677H, P681H/R, I692V, A701V, T7161, F888L, D950N, S982A, T1027I, Q1071H and D1118H;

In an embodiment, the S protein monomer comprises residues 1-1208 of the amino acid sequence SEQ ID NO:1 or a sequence at least 90% identical thereto. In an embodiment, the S protein monomer comprises residues 1-1208 of the amino acid sequence SEQ ID NO:2 or a sequence at least 90% identical thereto. In an embodiment, the S protein monomer comprises residues 1-1208 of the amino acid sequence SEQ ID NO:3 or a sequence at least 90% identical thereto.

In an embodiment, the S protein monomer comprises residues 1-1237 the amino acid sequence of SEQ ID NO:1 or a sequence at least 90% identical thereto.

In an embodiment, the S protein monomer comprises residues 1-1237 of the amino acid sequence of SEQ ID NO:3 or a sequence at least 90% identical thereto.

In an embodiment, the S protein monomer comprises residues 1-1256 the amino acid sequence of SEQ ID NO:1 or a sequence at least 90% identical thereto.

In an embodiment, the S protein monomer comprises residues 1-1256 of the amino acid sequence of SEQ ID NO:3 or a sequence at least 90% identical thereto.

In an embodiment, the S protein monomer comprises the amino acid sequence of SEQ ID NO: 6 or a sequence at least 90% identical thereto.

In an embodiment, the S protein monomer comprises the amino acid sequence of SEQ ID NO: 8 or a sequence at least 90% identical thereto.

In an embodiment, the S protein monomer comprises the amino acid sequence of SEQ ID NO: 25 or a sequence at least 90% identical thereto.

In an embodiment, the S protein monomer comprises the amino acid sequence of SEQ ID NO: 26 or a sequence at least 90% identical thereto.

In an embodiment, the S protein monomer comprises a sequence encoding the transmembrane domain of a coronavirus. In an embodiment, the S protein monomer comprises a sequence encoding the transmembrane domain of SARS-COV2. In an embodiment, the transmembrane domain comprises residues 1217 to 1237 of the amino acid sequence SEQ ID NO: 1 or a sequences at least 90% identical thereto. In an embodiment, the transmembrane domain comprises residues 1217 to 1237 of the amino acid sequence SEQ ID NO:3 or a sequences at least 90% identical thereto. In an embodiment, the transmembrane domain comprises residues 1209 to 1256 of the amino acid sequence SEQ ID NO:1 or a sequences at least 90% identical thereto. In an embodiment, the transmembrane domain comprises residues 1209 to 1256 of the amino acid sequence SEQ ID NO:3 or a sequences at least 90% identical thereto.

In an embodiment, the S protein monomer does not comprise a sequence encoding the transmembrane domain of a coronavirus.

In an embodiment, the S protein monomer comprises the 2P mutation as described herein. In an embodiment, the S protein monomer does not comprise the 2P mutation as described herein.

In an embodiment, the S protein monomer comprises the amino acid sequence of one or more of the VOC and or VOI mutations as described herein.

In an embodiment, the S protein monomer comprises S protein residues 1-1208 of a SARS-CoV-2 VOC. In an embodiment, the S protein monomer comprises S protein residues 1-1208 of a SARS-CoV-2 VOI. In an embodiment, the S protein monomer comprises S protein residues 1-1208 of a SARS-CoV-2 VHC.

In an embodiment, the S protein monomer does not comprise a trimerization sequence. In an embodiment, the S protein monomer does not comprise a transmembrane domain sequence. In an embodiment, the S-protein monomer does not comprise a foldon sequence. In an embodiment, the S-protein monomer does not comprise FHA.

### Structural Modifications

Structural modification to the alanine cavity is effected using one or more of: amino acid substitutions disulphide bond, hydrogen bond, pi stacking (TT-TT stacking), salt bridge, van der Waals interactions, use of hydrophobic residue substitutions or additions, or proline stabilisation within the S protein. In one embodiment, structural modification to the alanine cavity is effected by amino acid substitutions of one or more amino acids forming the alanine cavity. In an embodiment, the structural modification is the substitution of one or more amino acids to a more hydrophobic amino acid. In an embodiment, the structural modification is the substitution of one or more amino acids in the coiled-coiled region to a more hydrophobic amino acid.

In an embodiment, one or two or three of the S protein monomers in the S protein trimer comprise the substitution of one or more amino acids in the coiled-coiled region to a more hydrophobic amino acid. In an embodiment, one of the S protein monomers in the S protein trimer comprise the substitution of one or more amino acids in the coiled-coiled region to a more hydrophobic amino acid. In an embodiment, two of the S protein monomers in the S protein trimer comprise the substitution of one or more amino acids in the coiled-coiled region to a more hydrophobic amino acid. In an embodiment, three of the S protein monomers in the S protein trimer comprise the substitution of one or more amino acids in the coiled-coiled region to a more hydrophobic amino acid.

### Substitutions of Amino Acids in the Alanine Cavity

In an embodiment, the structural modification creates an artificial hydrophobic core in the coiled-coil region. In an embodiment the structural modification creates an artificial hydrophobic core comprising the residues of the alanine cavity. In an embodiment, the structural modification creates an artificial hydrophobic core in the alanine cavity. In an embodiment, the amino acids at positions 1016 and 1020 contribute to the formation of the artificial hydrophobic core.

In an embodiment, the artificial hydrophobic core is created by substituting an amino acid in the coiled-coil region with a more hydrophobic amino acid. In one embodiment, polar residues are replaced with bulkier hydrophobic residues.

As used herein “a more hydrophobic amino acid” refers to an amino acid that is more hydrophobic than the amino acid present in the position of the coronavirus strain that is being substituted. For example, if the amino acid being modified/substituted is an alanine, it may be substituted with a more hydrophobic amino acid e.g. isoleucine, leucine, methionine, valine, phenylalanine, tyrosine and tryptophan.

The hydrophobicity index is a measure of the relative hydrophobicity, or how soluble an amino acid is in water and is described for example in Sereda et al (1994) and Monera et al., (1995). The hydrophobicity of different amino acids at pH 2 and pH7 normalised so that the most hydrophobic residue is given at a value of 100 relative to glycine (0 value) is provided in the table below.

Amino acid hydrophobicity

| | |
|---|---|
| At pH 2 | At pH 7 |

Very Hydrophobic

| | | | |
|---|---|---|---|
| Leu | 100 | Phe | 100 |
| Ile | 100 | Ile | 99 |
| Phe | 92 | Trp | 97 |
| Trp | 84 | Leu | 97 |
| Val | 79 | Val | 76 |
| Met | 74 | Met | 74 |

Hydrophobic

| | | | |
|---|---|---|---|
| Cys | 52 | Tyr | 63 |
| Tyr | 49 | Cys | 49 |
| Ala | 47 | Ala | 41 |

Neutral

| | | | |
|---|---|---|---|
| Thr | 13 | Thr | 13 |
| Glu | 8 | His | 8 |
| Gly | 0 | Gly | 0 |
| Ser | −7 | Ser | −5 |
| Gln | −18 | Gln | −10 |
| Asp | −18 | | |

Hydrophilic

| | | | |
|---|---|---|---|
| Arg | −26 | Arg | −14 |
| Lys | −37 | Lys | −23 |
| Asn | −41 | Asn | −28 |
| His | −42 | Glu | −31 |
| Pro | −46 | Asp | −55 |

In an embodiment, the more hydrophobic amino acid is a hydrophobic amino acid. In an embodiment, the hydrophobic amino acid is an aliphatic hydrophobic amino acid. In an embodiment, the hydrophobic amino acid is an aromatic hydrophobic amino acid.

In an embodiment, at least one amino acid in the coiled-coil region of a S protein monomer in the S protein trimer is substituted with a more hydrophobic amino acid. In an embodiment, at least one S protein monomer in the S protein trimer comprises the substitution. In an embodiment, at least two S protein monomers in the S protein trimer comprise the substitution. In an embodiment, three S protein monomers in the S protein trimer comprise the substitution.

In an embodiment, at least two amino acids in the coiled-coil region of a S protein monomer in the S protein trimer are substituted with a more hydrophobic amino acid. In an embodiment, at least one S protein monomer in the S protein trimer comprises the substitutions. In an embodiment, at least two S protein monomers in the S protein trimer comprise the substitutions. In an embodiment, three S protein monomers in the S protein trimer comprise the substitutions.

In an embodiment, the at least one amino acid or at least two amino acids are in position a and/or d of the heptad repeat motif of the coiled-coil region of the S protein monomers. The locations of position a and din the in the heptad repeat motif are shown in FIG. 1B. For SARS-COV-2, positions a and d correspond to amino acids 988, 991, 995, 998 1002, 1005, 1009, 1013, 1016, 1020, 1023, 1027, 1031 of SEQ ID NO:1. In an embodiment, a substitution with a more hydrophobic amino acid occurs at position 1016. In an embodiment, a substitution with a more hydrophobic amino acid occurs at position 1020. In an embodiment, a substitution with a more hydrophobic amino acid occurs at position 1016 and 1020. In an embodiment, A1016 or A1020 are substituted with leucine, valine, Isoleucine or phenylalanine. In an embodiment, A1016 is substituted with leucine (A1016L), valine (A1016V) or isoleucine (A1016I). In an embodiment, A1020 is substituted with isoleucine (A1020I). In an embodiment, A1016 is substituted with leucine (A1016L) or valine (A1016V) and A1020 is substituted with isoleucine (A1020I). In an embodiment, A1016 is substituted with leucine (A1016L). In an embodiment, A1016 is substituted with valine (A1016V). In an embodiment, A1020 is substituted with isoleucine (A1020I). In an embodiment, A1020 is not substituted with tryptophan (W). In an embodiment, A1016 is substituted with valine and A1020 is substituted with isoleucine (referred to herein as “A1016V/A1020I” or “1016/20VI” or “VI”).

In an embodiment, the more hydrophobic amino acid comprises one or more of the following properties: i) a hydrophobicity greater than alanine; ii) a hydrophobic amino acid that is larger than alanine; ii) a hydrophobicity greater than 47 at a pH of 2; iii) a hydrophobicity greater than 41 at a pH of 7; and iv) is selected from: isoleucine, leucine, methionine, valine, phenylalanine, tyrosine and tryptophan. In an embodiment, the amino acid is selected from: isoleucine, leucine, valine. In an embodiment, the amino acid is isoleucine. In an embodiment, the amino acid is leucine. In an embodiment, the amino acid is valine. In an embodiment, the amino acid is methionine. In an embodiment, the amino acid is phenylalanine. In an embodiment, the amino acid is tyrosine. In an embodiment, the amino acid is tryptophan.

In an embodiment, the more hydrophobic amino acid comprises a hydrophobicity greater than alanine. In an embodiment, the more hydrophobic amino acid is larger than alanine. In an embodiment, the more hydrophobic amino acid comprises a hydrophobicity greater than 47 at a pH of 2. In an embodiment, the more hydrophobic amino acid comprises a hydrophobicity greater than 41 at a pH of 7. In an embodiment, the more hydrophobic amino acid is selected from: isoleucine, leucine, methionine, valine, phenylalanine, tyrosine and tryptophan. In an embodiment, the more hydrophobic amino acid is isoleucine. In an embodiment, the more hydrophobic amino acid is leucine. In an embodiment, the more hydrophobic amino acid is methionine. In an embodiment, the more hydrophobic amino acid is valine. In an embodiment, the more hydrophobic amino acid is phenylalanine. In an embodiment, the more hydrophobic amino acid is tyrosine. In an embodiment, the more hydrophobic amino acid is tryptophan.

For the avoidance of doubt, where optimal hydrophobicity has been achieved by amino acid substitution of the alanine cavity (including A1016 and A1020), further conservative amino acid mutations could be made to the region without affecting the desirable performance of the spike protein as described herein. Conservative amino acid substitutions are known in the art.

### Further Modifications/Further Stabilising Modifications

In an embodiment, the CoV vaccine as described herein comprises one or more further modifications to enhance one or more of the: stability, immunogenicity, expression and purification of the S protein trimer. Where the vaccine is a polynucleotide based vaccine, further modifications to enhance the in vivo stability and expression of a polynucleotide comprising the S protein encoding sequence or its soluble forms.

In an embodiment, the further modification to the antigen or its encoding molecule is selected from a proline stabilisation, furin cleavage site, a trimerization sequence, a repeat or a spacer, or nucleotide sequences encoding same.

In an embodiment, the proline stabilisation modification is 986P and/or 987P. The presence of both 986P and 987P in the S protein trimer is referred to as the “2P” modification.

In an embodiment, the further modification is the insertion of a furin cleavage site. In an embodiment, the mutation PG682SAS is introduced to insert a furin cleavage site (e.g. PG682SAS replaces RR682RAR in delta and PG682SAS replaces HR682RAR in omicron). In an embodiment, the further modification is the addition of FHA. In an embodiment, the further modification is the addition of a purification tag.

### Antigen Combinations

In one embodiment, the subject modified S antigen elicits broadly neutralising immune responses against the strain from which it is derived and one or more other strains circulating in the community. In another aspect, the antigen or vaccine comprising the antigen or encoding sequence, delivers one or multiple antigens of interest to a subject and induces an effective functional and polyfunctional immune response against homologous or heterologous strains including for example T-cell and antibody responses. In one embodiment, coronavirus antigens from one or more strains are selected from one or two or three or four of spike, nucleocapsid, membrane and envelope proteins. In one embodiment, amino acid and/or nucleotide sequences encoding SARS-CoV proteins two, three or four of N, M, E and S are employed. In an illustrative embodiment, N, M, E and S are employed. In an embodiment, one or two or multiple different variants of SARS-CoV are combined. In an embodiment, multiple variants and multiple antigens are employed. In one embodiment, the antigen or vaccine comprising the antigen or encoding sequence is administered with one or more B-cell and/or T-cell epitopes.

Cell lines capable of expressing the herein disclosed modified S antigen together with one or more of N, M, E antigens, or their encoding sequences are contemplated herein.

Combined administration of the subject antigen together with one or more of N, M, E antigens or their encoding sequences for one or two or multiple variants of interest/concern may be at the same time or spaced apart, in the same or different composition, optionally with combined use of protein and nucleic acid administration protocols at the same time or sequentially.

Spike RBD-only vaccine antigens (e.g., 319-545) are also contemplated in protein or nucleic acid vaccine formats. In one embodiment, administration of the present modified S antigen is combined with an antigen representing the RBD-only portion of the coronavirus in protein or nucleic acid form (eg, mRNA). The RBD domain can be administered in monomeric, dimeric, or multimeric form and may include immunopotentiating elements such as the Fc fragment of human IgG.

In another embodiment, the modified RBD of the present modified S antigen trimer is produced or administered as an RBD-only antigen in a suitable vaccine format. Specifically, the structural modification to the coiled-coil of S2 beneficially alters the structure and immunogenicity of the S1 RBD region and accordingly, production or administration of an RBD form based on the RBD form produced by the subject modified S antigen is contemplated.

In another embodiment, the modified S1 of the present modified S antigen trimer is produced or administered as an S1-only antigen in a suitable vaccine format. Specifically the structural modification to the coiled-coil of S2 alters the structure and immunogenicity of the S1 region and accordingly, production or administration of an S1 form based on the form produced by the subject modified S antigen is contemplated. In one embodiment, part of the S1 region absent the RBD is produced or administered. The S1 domain, or S1 minus the RBD domain, may be administered in monomeric, dimeric, or multimeric form and may include immunopotentiating elements, such as the Fc fragment of human IgG.

### Antibody Responses

S protein is the main protein used as a target antigen in COVID-19 vaccines. Theoretically, antibodies can target the S protein to inhibit virus infection at multiple stages during the virus entry process. The RBD is the major target for neutralising antibodies (NAbs) that interfere with viral receptor binding. To date, most of the potent NAbs to SARS-CoV-2 target the RBD. In addition, NAbs targeting the N-terminal domain have been reported in SARS-CoV-2 and MERS-CoV infection making it another potential target for inclusion in a vaccine. The S2 subunit is also a potential target for neutralising antibodies that interfere with the structural rearrangement of the S protein and the insertion of the fusion protein required for virus-host membrane fusion.

As used herein “broadly neutralizing antibodies” refers to antibodies that provide cross protection against at least one but preferably multiple coronavirus variants (e.g. multiple SARS-COV-2 variants). In an embodiment, at least one of the variants is a VOC or VOI or VHC.

NAbs are referred to as functional antibodies because they have a functional anti-virus effect.

The ability of vaccines to elicit NAbs or effective immune responses against heterologous strains or emerging variants of concern is a major factor influencing the successful roll out of a vaccine program against SARS-CoV-2.

The ability of vaccines to elicit NAbs or effective immune responses against homologous and heterologous strains or emerging variants of concern is a major factor influencing the successful roll out of a vaccine program against SARS-CoV-2. The present application enables the production and use of coronavirus S antigen mutants as described herein that elicit an enhanced immunogenicity to a broader range of variants including ancestral and naturally occurring and emerging variants of concern.

A diminished vaccine efficacy against variants of concern has been observed and can be assessed, for example, by screening for NAbs in vaccine immunised subjects against one or more strains or variants of the virus. As determined herein, in one embodiment the structurally modified antigen enabled herein is capable of generating, in a subject, a functional antibody response or neutralising antibody titre against heterologous strains including variants of concern that is at least as good as that against homologous strains.

In one embodiment, the antigen as defined herein elicits a neutralising antibody titre against a VOC that is at the same or a similar level as that generated against one or more non-variant strains.

At least 100% of the homologous antibody titre. In one embodiment, the titre is more than 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or more than 99% of the corresponding homologous titre.

### Methods of Producing the Coronavirus Vaccine Antigen

Antigens as described herein may be produced by recombinant or synthetic routes as known in the art.

In one embodiment, a nanoparticle is provided comprising an antigen as described herein fused to a polyhedrin targeting peptide from CPV or other suitable virus. Other nanoparticles are known in the art and include SOR particles, luminazine synthase particles, pyruvate dehydrogenase particles.

The antigen may be linked to a carrier or nanoparticle for increased immunogenicity. Suitable carriers are known in the art.

Viral like particles offer some of the structural complexity/advantages of viral surface proteins to antigens and may be derived from any suitable viruses. As used herein “a virus like particle” refers to vaccines comprising viral surface proteins but lack the viral genome and one or more structural proteins. Human and hepadnavirus HBV are good examples. VLPs comprising the antigen may form for example spontaneously upon recombinant expression of the protein and may be characterised using conventional technology.

Vaccines in the form of liposomes are encompassed. The term “liposome” herein refers to uni- or multilamellar lipid structures enclosing an aqueous interior. Lipids which are capable of forming liposomes include all substances having fatty or fat-like properties. Dynamic laser light scattering is a method used to measure the size of liposomes well known to those skilled in the art. An extensive description of adjuvants can be found in Cox and Coulter, “Advances in Adjuvant Technology and Application”, in Animal Parasite Control Utilizing Biotechnology, Chapter 4, Ed. Young, W. K., CRC Press 1992, and in Cox and Coulter, Vaccine 15 (3): 248-256, 1997.

Antigen may be delivered in the form of viral or non-viral vectors. The term “vector” as used herein includes any transmitting moiety into which the antigen encoding sequence at least is inserted, including plasmid vectors, cosmid vectors, phage vectors such as lambda phage, virus-like particles, viral vectors such as such as adenoviruses, adeno-associated viruses (AAV), alphaviruses, flaviviruses, herpes simplex viruses (HSV), measles viruses, CMV, rhabdoviruses, retroviruses, lentiviruses, Newcastle disease virus (NDV), poxviruses, and picornaviruses or baculoviral vectors, or artificial chromosome vectors such as bacterial artificial chromosomes (BAC), yeast artificial chromosomes (YAC), or PI artificial chromosomes (PAC). Vectors include expression as well as cloning vectors. In an embodiment, the primary coronavirus vaccine regimen is a viral vector vaccine. As used herein “viral vector vaccines” use viral backbones to insert a SARS-CoV-2 gene, or a portion thereof into a host organism. These vaccines transmit genes into target cells, where the genes are expressed and the expressed gene can illicit an immune response. In one embodiment, the vector is a replicating vector. In an embodiment, the vector is a non-replicating vector (a vector that does not integrate into the host cell). In an embodiment, the vector is selected from an adenovirus, poxvirus, measles virus and a vesicular stomatitis virus.

Expression vectors comprise plasmids as well as viral vectors and generally contain a desired coding sequence and appropriate NA sequences necessary for the expression of the operably linked coding sequence in a particular host organism (e.g., bacteria, yeast, plant, insect, or mammal) or in in vitro expression systems. Cloning vectors are generally used to engineer and amplify a certain desired NA fragment and may lack functional sequences needed for expression of the desired DNA fragments.

In one embodiment, the vector is a viral vector or a non-viral vector. Viruses, which are useful as vectors include, but are not limited to, retroviruses, adenoviruses, adeno-associated viruses, herpes viruses and lentiviruses, and attenuated forms thereof, each of which have their own advantages and disadvantages as known in the art. Viral vectors specifically include without limitation an adenoviral vector and a poxviral vector. Typically, for viral vectors, about 5×10⁷ to 5×10¹² viral particles are administered, typically about 5×10⁹ to 5×10¹⁰ viral particles.

The antigen encoding sequence may be inserted into any suitable vector. The purpose of the vector is at least in one embodiment to transmit the encoding nucleic acid to the host environment to facilitate protein expression and presentation to the host immune response. Vectors may be replicating or non-replicating.

Expression vectors generally include transcriptional and translational regulatory nucleic acid operably linked to the nucleic acid molecule encoding the antigen. “Operably linked” in this context means that the transcriptional and translational regulatory DNA is positioned relative to the coding sequence of the antigen in such a manner that transcription is initiated. Generally, this will mean that the promoter and transcriptional initiation or start sequences are positioned 5′ to the protein coding region. The transcriptional and translational regulatory nucleic acid will generally be appropriate to the cell used to express the exogeneous protein; for example, transcriptional and translational regulatory nucleic acid sequences from mammalian cells, and particularly humans, are preferably used to express the protein in mammals and humans. Numerous types of appropriate expression vectors, and suitable regulatory sequences are known in the art.

The viral vector may comprise a vaccinia vector such as synthetic modified vectors based on the Copenhagen vaccinia vector or Modified Vaccinia Ankara (MVA). The viral vector May comprise MVA or a Copenhagen derivative as known in the art when used as a vaccine boost in a prime boost regime. The viral vector may comprise Adeno-associated virus (AAV) or lentivirus. The viral vector may be an attenuated viral vector. For example, essential genes for replication may be deleted and immunomodulatory molecules inserted.

Viral vectors may be maintained in BACs for ease of manipulation and the DNA encoding antigen may be linear or circular.

Non-viral vectors or attachments/conjugates include lipids, carbohydrates, proteins, peptides, nanoparticles, liposomes, viral-like particles, virosomes, emulsions. Amphipathic agents such as lipids may exist in aggregates as micelles, insoluble monolayers, liquid crystals or lamellar layers in aqueous solution.

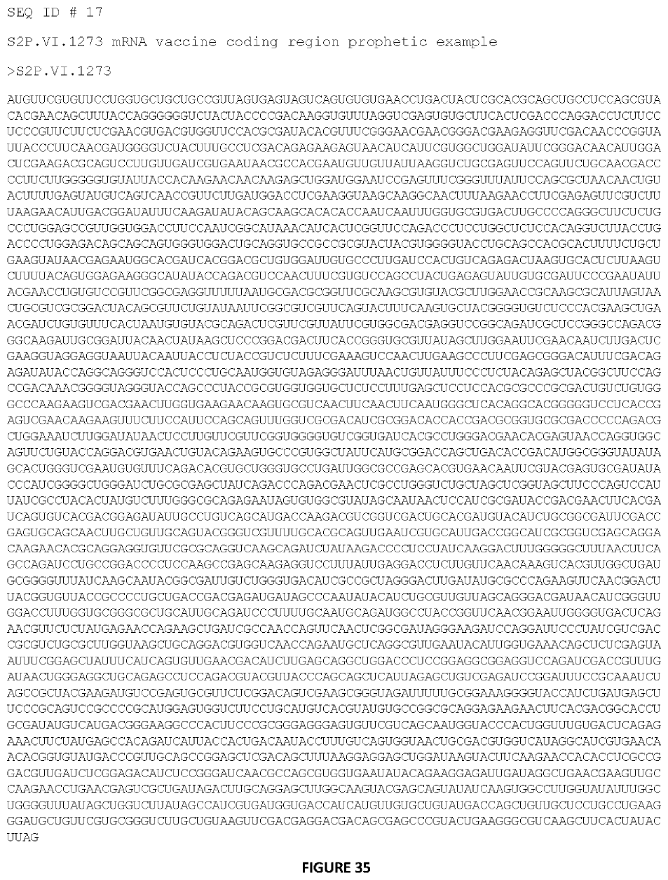

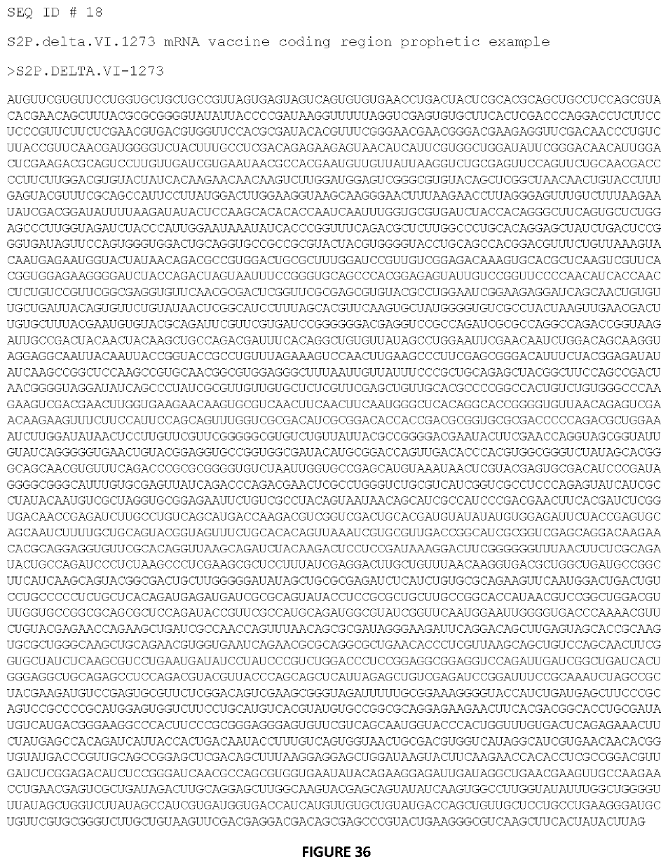

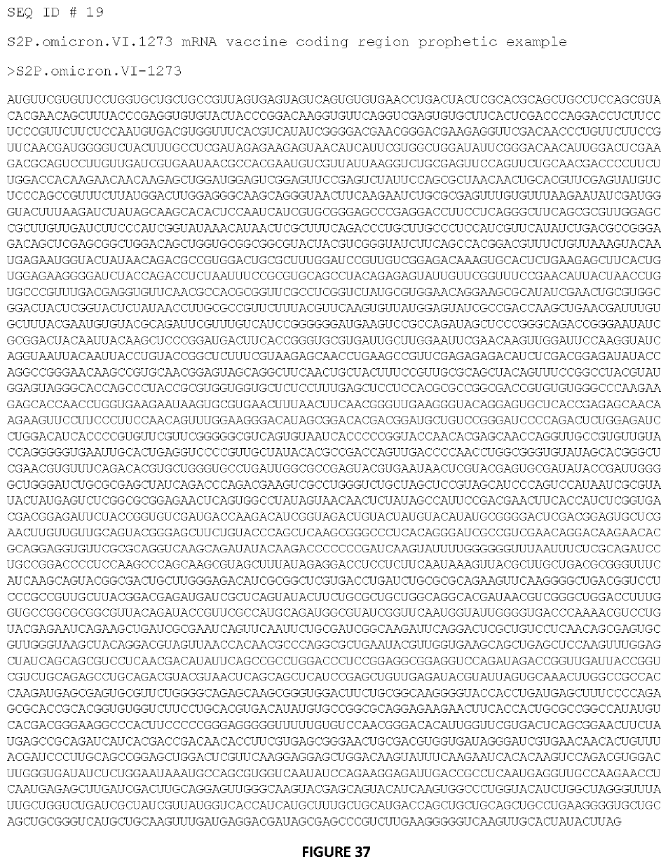

The antigen may be administered in the form of its encoding nucleic acid. A nucleic acid molecule as described herein may in any form such as DNA, cDNA, genomic DNA, or RNA, including in vitro transcribed RNA or synthetic RNA, mRNA or PNA or a mixture thereof. Nucleic acids include genomic DNA, cDNA, mRNA, recombinantly produced and chemically synthesized molecules and modified forms thereof. A nucleic acid molecule may be single stranded or double stranded and linear or closed covalently to form a circle. In an embodiment, the nucleic acid is 35 RNA. The RNA may be modified by stabilizing sequences, capping, and polyadenylation. RNA or DNA and may be delivered as plasmids to express antigen and induce immune responses. The RNA may be modified to enhance delivery via a lipid nanoparticle. The RNA may be modified to increase stability of the RNA molecule. Examples of RNA of the present invention are provided in FIGS. 35 to 37. RNA-based approaches are generally preferred and these can include amplifying or non-self amplifying constructs. A nucleic acid molecule is usually at least 10 bases in length, and may be single or double stranded. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polynucleotides or polypeptides.

In some embodiments, an RNA encoding the antigen is administered. In some embodiments, the RNA encodes an antigen comprising a coronavirus transmembrane domain as described herein. In some embodiments, the RNA encodes an antigen lacking a coronavirus transmembrane domain as described herein. In some embodiments, the RNA encodes an antigen comprising a trimerization domain as described herein. In some embodiments, the RNA encodes an antigen lacking a trimerization domain as described herein.

In some embodiments the polynucleotide to be administered by transient in vivo transfection is a chemically modified RNA in which a proportion (e.g., 10%, 30%, 50%, or 100%) of at least one type of nucleotide, e.g., cytosine, is chemically modified to increase its stability in vivo. For example, in some cases modified cytosines are 5 methylcytosines. Such polynucleotides are particularly useful for delivery/transfection to cells in vivo, especially when combined with a transfection/delivery agent. In some cases, a chemically modified RNA is a chemically modified RNA in which a majority of (e.g., all) cytosines are 5-methylcytosines, and where a majority (e.g., all) of uracils are pseudouracils. In some embodiments, non-native cysteines are engineered to create di-sulphide bonds (e.g. via recombinant genentic technologies). The synthesis and use of such modified RNAs are described in, e.g., WO 2011/130624. Methods for in vivo transfection of DNA and RNA polynucleotides are known in the art as summarised in, e.g., Liu et al (2015) and Youn et al (2015).

The term “RNA” relates to a molecule which comprises ribonucleotide residues and preferably being entirely or substantially composed of ribonucleotide residues. “Ribonucleotide” relates to a nucleotide with a hydroxyl group at the 2′-position of a B-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

Accordingly, in one embodiment, the G/C content of the coding region of the nucleic acid coding region is modified, particularly increased, compared to the G/C content of the coding region of its particular wild type coding sequence, i.e. the unmodified mRNA. The encoded amino acid sequence of the mRNA is preferably not modified compared to the coded amino acid sequence of the particular wild type mRNA.

An optimised mRNA based composition could comprise a 5′ and 3′ non translated region (5′-UTR, 3′-UTR) that optimise translation efficiency and intracellular stability as known in the art and an open reading frame encoding the S protein. In one embodiment, removal of uncapped 5′-triphosphates can be achieved by treating RNA with a phosphatase. RNA may have modified ribonucleotides in order to increase its stability and/or decrease cytotoxicity. For example, in one embodiment, in the RNA, 5-methylcytidine is substituted partially or completely, for cytidine. Alternatively or additionally, pseudouridine is substituted partially or completely, preferably completely, for uridine. These modification may also reduce indiscriminate immune inactivation which may hinder translation of the RNA. In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap. The term “5′-cap” includes a 5′-cap analog that resembles the RNA cap structure and is modified to possess the ability to stabilize RNA and/or enhance translation of RNA. Providing an RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

A further modification of RNA may be an extension or truncation of the naturally occurring UTR such as the X-region tail or an alteration of the 5′- or 3 ‘-untranslated regions (UTR) such as introduction of a UTR which is not related to the coding region of said RNA, for example, the exchange of the existing 3’-UTR with or the insertion of one or more, preferably two copies of a 3′-UTR derived from a globin gene, such as alpha2-globin, alphaI-globin, beta-globin. RNA having an unmasked poly-A sequence is translated more efficiently than RNA having a masked poly-A sequence.

The term “poly(A) tail” or “poly-A sequence” relates to a sequence of adenyl (A) residues which may be located on the 3′-end of a RNA molecule and “unmasked poly-A sequence” means that the poly-A sequence at the 3′ end of an RNA molecule ends with an A of the poly-A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of the RNA it may be modified so as to be present in conjunction with a heterologous poly-A sequence, preferably having a length of 10 to 500, more preferably 30 to 300, even more preferably 65 to 200 and especially 100 to 150 adenosine residues. In an especially preferred embodiment the poly-A sequence has a length of approximately 120 adenosine residues. To further increase stability and/or expression of the RNA used according to the invention, the poly-A sequence can be unmasked.

In addition, incorporation of a 3′-non translated region (UTR) into the 3′-non translated region of an RNA molecule can result in an enhancement in translation efficiency. A synergistic effect may be achieved by incorporating two or more of such 3′-non translated regions. The 3′-non translated regions may be autologous or heterologous to the RNA into which they are introduced. In one particular embodiment the 3′-non translated region is derived from the human β-globin gene.

A combination of the above described modifications, i.e. optionally incorporation of a poly-A sequence, unmasking of the poly-A sequence and incorporation of one or more 3′-non translated regions, has a synergistic influence on the stability of RNA and increase in translation efficiency.

In order to increase expression of the RNA it may be modified within the coding region so as to increase the GC-content to increase mRNA stability and to perform a codon optimization and, thus, enhance translation in cells. Modified mRNA may be synthesised enzymatically and packaged into nanoparticles such as lipid nanoparticles and administered, for example intramuscularly. Self-replicating RNA or protamine complexed RNA approaches have also been shown to generate immune responses against viral infections.

The nucleic acid molecule can be entrapped in microcapsules prepared, for example, by coacervation techniques or by interfacial polymerization, in colloidal drug delivery systems (e.g., liposomes, microspheres, microemulsions, nanoparticles and nanocapsules), or in macroemulsions. Such techniques are known in the art and disclosed in Remington, the Science and Practice of Pharmacy, 20th Edition, Remington, J., ed. (2000).

Various approaches for systemic administration of nucleic acids as nanoparticles or colloidal systems are known. In non-viral approaches, cationic liposomes are used to induce DNA/RNA condensation and to facilitate cellular uptake. The cationic liposomes usually consist of a cationic lipid, like DOTAP, and one or more helper lipids, like DOPE. So-called ‘lipoplexes’ can be formed from the cationic (positively charged) liposomes and the anionic (negatively charged) nucleic acid. In the simplest case, the lipoplexes form spontaneously by mixing the nucleic acid with the liposomes with a certain mixing protocol, however various other protocols may be applied. In one embodiment, nanoparticulate RNA formulations such as RNA lipoplexes, are produced with defined particle size wherein the net charge of the particles is close to zero or negative. For example, electro-neutral or negatively charged lipoplexes from RNA and liposomes lead to substantial RNA expression in spleen or immune cells after systemic administration as disclosed in WO2013/143683. In one embodiment, the nanoparticles comprise at least one lipid. In one embodiment, the nanoparticles comprise at least one cationic lipid. The cationic lipid can be monocationic or polycationic. Any cationic amphiphilic molecule, eg, a molecule which comprises at least one hydrophilic and lipophilic moiety is a cationic lipid within the meaning of the present invention. In one embodiment, the positive charges are contributed by the at least one cationic lipid and the negative charges are contributed by the RNA. In one embodiment, the nanoparticles comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid.

In one embodiment, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof.

In one embodiment, the at least one helper lipid comprises 1,2-di-(9Z-octadecenoyl)-sn-glycero-3-phosphoethanolamine (DOPE) or analogs or derivatives thereof, cholesterol (Choi) or analogs or derivatives thereof and/or 1,2-dioleoyl-sn-glycero-3-phosphocholine (DOPC) or analogs or derivatives thereof.

In one embodiment, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In one embodiment, in this ratio, the molar amount of the cationic lipid results from the molar amount of the cationic lipid multiplied by the number of positive charges in the cationic lipid. In the nanoparticles described herein the lipid may form a complex with and/or may encapsulate the RNA. In one embodiment, the nanoparticles comprise a lipoplex or liposome. In one embodiment, the lipid is comprised in a vesicle encapsulating said RNA. The vesicle may be a multilamellar vesicle, an unilamellar vesicle, or a mixture thereof. The vesicle may be a liposome.

A lipid nanoparticle (LNP) is generally known as a nanosized particle composed of a combination of different lipids (an aqueous volume is encapsulated by amphipathic lipid bilayers e.g., single; unilamellar or multiple; multilamellar). Many different types of lipids may be included in LNP. In some embodiments, the lipids may be one or more of an ionisable lipid, a phospholipid, a structural lipid, neutral lipid and a PEG lipid. For example, the mRNA is encapsulated in a LNP. In another example, the mRNA is bound to the LNP. For example, the mRNA is absorbed on the LNP.

Methods of preparing LNP are known to a person skilled in that art and are described, for example, in Huang et al (2021) and Schoenmaker et al (2021). As used herein, the term “ionisable lipid” or “ionisable lipids” shall refer to a lipid having at least one protonatable or deprotonatable group. For example, the lipid is positively charged at a pH at or below physiological pH (e.g. pH 7.4), and neutral at a second pH (e.g. at or above physiological pH). n an embodiment, the lipid nanoparticle comprises an ionisable lipid as described in Table 1 of Schoenmaker et al (2021).

Suitable ionisable lipids can have an anionic, cationic or zwitterionic hydrophilic head group. Exemplary phospholipids (anionic or zwitterionic) for use in the present disclosure include, for example, phosphatidylethanolamines, phosphatidylcholines, phosphatidylserines, and phosphatidylglycerols. In one example, the lipid is a cationic lipid. Exemplary cationic lipids include, but are not limited to, dioleoyl trimethylammonium propane (DOTAP), 1,2-distearyloxy-N,N-dimethyl-3-aminopropane (DSDMA), 1,2-dioleyloxy-N, Ndimethyl-3-aminopropane (DODMA), 1,2-dilinoleyloxy-N,N-dimethyl-3-aminopropane (DLinDMA), 2,5-bis((9z,12z)-octadeca-9,12,dien-1-yloxyl)benzyl-4-(dimethylamino)butanoate (LKY750). In one example, the phospholipid is 2,5-bis((9z,12z)-octadeca-9,12,dien-1-yloxyl)benzyl-4-(dimethylamino) butanoate (LKY750). Exemplary zwitterionic lipids include, but are not limited to, acyl zwitterionic lipids and ether zwitterionic lipids, such as dipalmitoylphosphatidylcholine (DPPC), dioleoylphosphatidylcholine (DOPC) and dodecylphosphocholine. The lipids can be saturated or unsaturated. In an embodiment, the lipid nanoparticle does not comprise a cationic lipid.

A person skilled person in the art will appreciate that reference to a PEGylated lipid is a lipid that has been modified with polyethylene glycol. Exemplary PEGylated lipids include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid includes PEG-c-DOMG, PEG-DMG, PEG-DLPE, PEG-DMPE, PEG-DPPC, a PEG-DSPE lipid and combinations thereof.

Suitable neutral or zwitterionic lipids for use in the present disclosure will be apparent to the skilled person and include, for example, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. The lipids can be saturated or unsaturated.

Exemplary structural lipids include, but are not limited to, cholesterol fecosterol, sitosterol, campesterol, stigmasterol, brassicasterol, ergosterol, tomatidine, tomatine, ursolic acid and alpha-tocopherol. In an embodiment, the structural lipid is a sterol. In an embodiment, the structural lipid is cholesterol. In an embodiment, the structural lipid is campesterol.

### Compositions, Routes of Administration and Doses

A person skilled in the art will appreciate that the coronavirus vaccine antigen as described herein, or the vectors or polynucleotides encoding a coronavirus vaccine antigen as described herein may be formulated into a pharmaceutical composition. In an embodiment, the pharmaceutical composition is a vaccine composition.

In an aspect, the specification provides a pharmaceutical composition comprising a polynucleotide comprising a sequence of nucleotides encoding a coronavirus S protein trimer antigen wherein the S protein trimer is modified to comprise a structural modification which reduces the size of the alanine cavity in the coiled-coil region of the S protein trimer as described herein.

In an aspect, the specification provides a pharmaceutical composition comprising a polynucleotide comprising a sequence of nucleotides encoding a coronavirus S protein trimer antigen wherein at least one amino acid in the region of the S protein monomer forming the coiled-coil of the S protein trimer is substituted with a more hydrophobic amino acid, as described herein.

Such compositions may include the coronavirus vaccine antigen, vector or polynucleotide as described herein and one or more pharmaceutically acceptable carriers. Remington's Pharmaceutical Sciences, by E. W. Martin, Mack Publishing Co., Easton, Pa., 19th Edition, 1995, describes compositions and formulations suitable for pharmaceutical delivery of the disclosed immunogens. In general, the nature of the carrier will depend on the particular mode of administration being employed. For instance, parenteral formulations usually comprise injectable fluids that include pharmaceutically and physiologically acceptable carriers such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

In an embodiment, the composition comprises a vaccine antigen as described herein. In an embodiment, the composition comprises a vector as described herein. In an embodiment, the composition comprises a polynucleotide as described herein. In an embodiment, the polynucleotide is a DNA. In an embodiment the polynucleotide is an RNA. In an embodiment, the polynucleotide is an mRNA.

In an embodiment, the composition comprises a lipid nanoparticle. In an embodiment, the lipid nanoparticle encapsulates a polynucleotide as described herein.

In an embodiment, when the composition is a vaccine composition it may comprise one or more other epitopes for eliciting an immune response e.g. B-cell and/or T-cell epitopes.

In an embodiment, the composition is formulated to be compatible with its intended route of administration, e.g., local or systemic. Examples of routes of administration include intradermal, subcutaneously, intravenously, intra-arterially, intraperitoneal, intranasal, sublingual, tonsillar, orally, pulmonary, topical or other parenteral and mucosal routes.

In an embodiment, the composition is formulated to be stable at refrigerator temperature. In an embodiment, the composition is formulated so that it is suitable for transportation and/or storage at refrigerator temperature. In an embodiment, refrigerator temperature is about 3° C. to about 17° C., or about 4° C. to about 10° C., or about 4° C. In an embodiment, the composition is formulation to be stable at room temperature. In an embodiment, room temperature is about 18° C. to about 24° C., or about 20° C. to about 23° C., or about 23° C. In an embodiment, the composition is formulated so that is suitable for non-cold chain transportation and/or storage. In an embodiment, the composition is formulated so that is suitable for room temperature storage and/or transpiration. In an embodiment, the composition is formulated so that is suitable for transportation and/or storage at temperatures higher than room temperature e.g. about 25° C. to 40° C. (for countries where cold chain and low temperature storage and transportation trains are not available.

Oral, nasal and pulmonary administration include administration via inhalation and sprays delivered to the aforementioned sites. Solutions or suspensions used for parenteral, intradermal, or subcutaneous application can include the following components: a sterile diluent, such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Compositions suitable for injectable use include sterile aqueous solutions (where water soluble) or dispersions, non-aqueous solutions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, sodium chloride can also be included in the composition. Prolonged absorption of the injectable compositions can be brought about by including in the composition an agent that delays absorption, such as aluminum monostearate or gelatin.

Sterile injectable solutions can be prepared by incorporating the coronavirus vaccine antigen, vector or polynucleotide as described herein in the required amount in an appropriate solvent or buffer with one or a combination of ingredients enumerated above, as required, followed by filtered sterilization. Generally, dispersions are prepared by incorporating the polynucleotide into a sterile vehicle, which contains a basic dispersion medium and the required other ingredients from those enumerated above. In the case of sterile powders for the preparation of sterile injectable solutions, suitable methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the coronavirus vaccine antigen, vector or polynucleotide as described herein can be incorporated with excipients and used in the form of sprays, tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The sprays, tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or sterotes; a glidant such as colloidal silicon dioxide; a sweetening agent such as sucrose or saccharin; or a flavouring agent such as peppermint, methyl salicylate, or orange flavouring.

Formulations suitable for administration by nasal inhalation include where the carrier is a solid, include a coarse powder having a particle size, for example, in the range of about 1 to about 500 microns, which is administered in the manner via a spray, nebuliser, inhaler or snuffed. Suitable formulations wherein the carrier is a liquid for administration by nebulizer, include aqueous or oily solutions of the agent. For administration by inhalation, the agent(s) can also be delivered in the form of drops or an aerosol spray from a pressured container or dispenser that contains a suitable propellant, e.g., a gas such as carbon dioxide, or a nebulizer. Such methods include those described in U.S. Pat. No. 6,468,798.

Formulations suitable for administration by oral inhalation include where the carrier is a solid, include a coarse powder having a particle size, for example, in the range of about 20 to about 500 microns, which is administered by oral inhalation from a container holding the powder held close to the mouth or where the carrier is a liquid for administration by nebulizer, which can include aqueous or oily solutions of the agent.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, drops, or suppositories. For transdermal administration, the coronavirus vaccine antigen, vector or polynucleotide as described herein are formulated into ointments, salves, gels, or creams, as generally known in the art.

Intra dermal delivery of vaccines by needle or needle free approaches offers advantages in terms of ease of administration and intradermal administration approaches that effectively target immunocompetent cells are contemplated. Liquid formulations may be provided in prefilled or non-prefilled syringes or needs such disposable-syringe jet injectors, hollow microneedles mounted on syringes, and needles adapted for intra-dermal delivery. Prefilled syringes with a single ID needle are commercially available. Alternatively, solid or biodegradable microneedles coated or impregnated with vaccine such as patches or other mini-needle/spike devices, or composed of vaccine may be employed. These are inserted into the dermal layers of the skin where either the vaccine coating is dissolved, or the microneedle itself dissolves in place. The vaccine antigen may be provided as a liquid or semi liquid formulation, or as a solid or powdered formulation. Jet-injectors operate by generating a high pressured stream, which flushes a liquid vaccine formulation into the deeper skin layers. However, approaches that deliver vaccines in a solid form may also prove to be promising. One such method is the ballistic approach, in which solid vaccine particles or vaccine-coated gold particles are accelerated towards the skin by needle-free devices, so that the particles are deposited in the epidermal and dermal layers of the skin.

Intramuscular administration, can be via any intramuscular method known by a person skilled in the art, including for example, intramuscular injection.

The compositions can also be prepared in the form of suppositories (e.g., with conventional suppository bases such as cocoa butter and other glycerides) or retention enemas for rectal delivery.

Compositions may include adjuvants. Immune responses to antigens can be enhanced if administered as a mixture with one or more adjuvants. Immune adjuvants typically function in one or more of the following ways: (1) immunomodulation (2) enhanced presentation (3) CTL production (4) targeting; and/or (5) depot generation.

Illustrative adjuvants that may or may not be included include: particulate or non-particulate adjuvants, complete Freund's adjuvant (CFA), aluminum salt-based adjuvant, emulsion based adjuvant, TLR agonists, ISCOMS, LPS derivatives such as MPL and derivatives thereof such as 3D-MPL also GLA, and AGP, mycobacterial derived proteins such as muramyl di- or tri-peptides, particular saponins from Quillaja saponaria, such as QS21, QS7, and ISCOPREP™ saponin, ISCOMATRIX™ adjuvant, and peptides, such as thymosin alpha 1. In addition to the saponin component, the adjuvant may comprises a sterol such as beta-sitosterol, stigmasterol, ergosterol, ergocalciferol and cholesterol. In some embodiments, the adjuvant is presented in the form of an oil-in-water emulsion, e.g. comprising squalene, alpha-tocopherol and a surfactant or in the form of a liposome. AddaVax is a squalene based oil in water nano emulsion based on the formulation of MF-59 that has been found useful in flu vaccines. The adjuvants AS03, MF59, and CpG 1018 have already been used in licensed vaccines. Other suitable adjuvants include lecithin and caromer homopolymers, Matrix M, ASO1, ALFQ. CpG mofits and co-stimulatory molecules including TLR agonists, B7, OX-40L, G-CSF are contemplated. Adjuvants are discussed in Liang et al Front. Immunol. 6 Nov. 2020.

In an embodiment, the composition comprises an adjuvant selected from one or more of an aluminium salt-based adjuvant, emulsion adjuvant, or a TLR agonist. Examples of such adjuvants are described for example in Liang et al (2020).

Suitable dosage ranges for intravenous administration of viral vectors, for example, is generally about 0.001 to 10 micrograms nucleic acid. Suitable dosage ranges for intranasal administration are generally about 0.01 pg/kg body weight to 10 mg/kg body weight. Effective doses may be extrapolated from dose-response curves derived from in vitro or animal model test systems. Suppositories generally contain active ingredient in the range of 0.5% to 10% by weight; oral compositions preferably contain 10% to 95% active ingredient.

A subject may receive one dose of the composition or two or three doses of the composition at scheduled intervals.

Antibodies generated against the subject antigen may be used in therapy or for screening. Antibody include an immunoglobulin, antigen-binding fragment, or derivative thereof, that specifically binds and recognises the antigen or an antigenic fragment thereof, or a dimer or multimer of the antigen. The term “antibody” is used herein in the broadest sense and encompasses various antibody structures, including but not limited to monoclonal antibodies, polyclonal antibodies, multi (and bi) specific antibodies, and antibody fragments. Examples of antibody fragments include but are not limited to Fv, Fab, Fab′, Fab′-SH, F(ab′).sub.2; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (Reference may be made to Kontermann and Dubel (Ed), Antibody Engineering, Vol 1-2, Ed., Springer Press, 2010).

The term epitope refers to particular peptide sequences on a molecule that are antigenic, such that they elicit a specific immune response. An epitope is the region of an antigen to which B and/or T cells respond. An antibody can bind to a particular antigenic epitope which may be formed both from contiguous amino acids or noncontiguous amino acids.

Methods of Prevent and/or Treatment

In an aspect, the present invention provides a method of preventing and/or treating a coronavirus infection in a subject.

As used herein, the term “prevention” or “prophylaxis” refers to reducing the likelihood of contracting or developing infection or a symptom thereof. Prevention need not be complete and does not imply that a subject will not eventually contract or develop the infection or a symptom thereof.

As used herein, the terms “treating” or “treatment” refers to at least partially obtaining a desired therapeutic outcome. In an embodiment, treatment comprises preventing or delaying the appearance of one or more symptoms of a CoV infection. In an embodiment, treatment comprises arresting or reducing the development of one or more symptoms of a CoV infection.

Reference to “subject” or “subjects” includes a subject susceptible to a coronavirus infection, or at risk of exposure to a coronavirus. The subject may be infected or uninfected, and may be symptomless or in need of treatment. In an embodiment, the subject is susceptible, or at risk of exposure to a SARS-CoV-2 infection. For example, the subject can be a mammal, avian, arthropod, chordate, amphibian or reptile. Exemplary subject's include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer), zoo animals (e.g. lion, tiger, bear), reservoir animals (e.g. bats, camels, pangolin). In an embodiment, the subject is a mammal. In one embodiment, the subject is human. In an embodiment, the human is a fetus, infant, child, early adult and adult. In one embodiment, the adult is an elderly adult. In an embodiment, the adult is one or more of greater than 60 years in age, greater than 65 years in age, greater than 70 years in age, greater than 75 years in age, greater than 80 years in age, greater than 85 years in age, greater than 90 years in age. In an embodiment, the subject has had a prior coronavirus infection. In an embodiment, the subject has had a prior SARS-CoV-2 infection. In an embodiment, the subject has received a primary coronavirus treatment regimen as described herein. In an embodiment, the subject has received a primary and a secondary coronavirus treatment regimen. In an embodiment, the subject has received a primary coronavirus treatment regimen, a secondary coronavirus treatment regimen and a tertiary coronavirus treatment regimen. In an embodiment, the subject is immunocompromised. In an embodiment, the subject has a respiratory condition.

In an aspect, the present invention provides a method of inducing an immune response to a coronavirus in a subject, the method comprising administering the vaccine as described herein. In an aspect, the present inventions provides a method of enhancing the immune response to coronavirus in a subject, the method comprising administering the vaccine as described herein.

In an aspect, the present invention provides a method of preventing or reducing the likelihood of a coronavirus infection in a subject, the method comprising administering the vaccine as described herein.

In an aspect, the present invention provides a method of preventing or, reducing the likelihood or severity of a symptom of a coronavirus infection in a subject, the method comprising administering the vaccine as described herein to a subject.

In an aspect, the present invention provides a method of reducing the severity and/or duration of a coronavirus infection in a subject, the method comprising administering the vaccine as described herein to a subject. As used herein, the phrase “reducing the severity of an infection”, or similar phrases, includes reducing one or more of the following in an individual: titer of a virus, duration of the virus infection, the harshness or duration of one or more symptoms of a coronavirus infection in a subject. As used herein, the phrase “duration of a coronavirus infection” refers to the time in which an individual has a CoV infection or a symptom caused by a CoV infection.

In an aspect, the present invention provides a method of preventing or reducing viral shedding in a human individual infected with a coronavirus, the method comprising administering the vaccine as described herein to a subject.

In an embodiment, the present invention provides a vaccine that is a primary vaccine regimen. As used herein a “primary vaccine regimen” is the first vaccine regimen administered to a subject to produce a response to a specific pathogen. In the context of SARS-CoV-2, a primary vaccine is the first vaccine regimen administered to a subject to produce an immune response to the ancestral strain and/or a variant thereof.

In an embodiment, the present invention provides a booster vaccine for a primary coronavirus vaccine regimen. In an embodiment, the present invention provides a booster vaccine for instances where a subject has received more than one prior coronavirus vaccine regimen. In an embodiment, the booster acts by enhancing the immune response elicited by the primary vaccine regimen. In an embodiment, the booster acts by enhancing the immune response to VOC or VOI or VHCs to which a lesser, little or no protective immune response is generated by the primary vaccine regimen. In an embodiment, the booster is administered at least 6 months, or at least 12 months, or at least 18 months, or at least 2 years, or at least 3 years or at least 5 years, or at least 6 years, or at least 7 years after the primary vaccine regimen. In an embodiment, the booster is administer sequentially or in combination with one or more other booster vaccines.

### Primary Vaccine Regimens

In some embodiments, the vaccine as described herein is administered after a subject has received a primary coronavirus vaccine regimen. A person skilled in the art will appreciate that primary coronavirus vaccine regime can be any coronavirus vaccine regimen that provides projection against a coronavirus infection. In an embodiment, the coronavirus vaccine is a SARS-CoV-2 coronavirus vaccine.

In an embodiment, the primary coronavirus vaccine regimen is selected from a: a) single dose vaccine regimen; b) two dose vaccine regimen; c) single dose of a two dose vaccine regimen or d) a combination thereof.

In an embodiment, the primary coronavirus vaccine regimen is selected from a: a) RNA based vaccine; b) DNA based vaccine; c) viral vector vaccine; d) inactivated vaccine; e) live attenuated vaccine; and f) protein subunit vaccine.

In an embodiment, the primary coronavirus vaccine regimen is an RNA based vaccine. As used herein an “RNA based vaccine” transmits instructions for the expression of a coronavirus antigen e.g. the S protein or a part thereof in a human cell via an RNA molecule such as mRNA.

In an embodiment, the primary coronavirus vaccine regimen is an DNA based vaccine. As used herein an “DNA based vaccine” transmits instructions for the expression of a coronavirus antigen e.g. the S protein or a part thereof in a human cell via a DNA molecule.

In an embodiment, the primary coronavirus vaccine regimen is a viral vector vaccine. In an embodiment, the vector is selected from an adenovirus, poxvirus, measles virus and a vesicular stomatitis virus.

In an embodiment, the primary coronavirus vaccine regimen is an inactivated virus vaccine. Inactivated vaccines are created by inactivating the virus with e.g. chemical UV light and/or heat so that they are no longer transmissible. Such vaccines are often desirable as they present several epitopes for immune recognition and generation of an immune response.

In an embodiment, the primary coronavirus vaccine regimen is a live attenuated coronavirus. As used herein “live attenuated” refers to

In an embodiment, the vaccine is a protein based vaccine, for example a protein subunit vaccine or a virus-like article. As used herein a “protein subunit vaccine” comprises immunogenic antigens that can stimulate a host immune response. Where the coronavirus is SARS-CoV-2 a protein subunit vaccine may comprise the S1 protein or a portion thereof, the RBD domain, the S2 protein or a portion thereof. In one embodiment, the protein based vaccine is a virus-like particles or nanoparticle.

### Combination Treatments

A coronavirus vaccine antigen or vaccine as described herein may be administered to a subject in combination with one or more further vaccine antigens or vaccines. The further vaccine antigens or vaccines may produce an immune response against an infectious pathogenic organism such as influenza, SARS-COV-2 or a specific VOC, VOI or VHC thereof. Administration may be in combination (at the same time) or sequential in either order.

### Kit, Device, Surface or Strip

The subject coronavirus antigen is captured on solid or semi-solid surfaces for assay purposes, including epidemiological, diagnostic, purification, drug-screening, vaccine screening applications etc. Many such applications and methods of immobilising antigen to surfaces are known in the art and encompassed.

As used herein, the term “complement” or “complementary” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides. As described herein and commonly known in the art the complementary (matching) nucleotide of adenosine is thymidine in DNA, or alternatively in RNA the complementary (matching) nucleotide of adenosine is uracil, and the complementary (matching) nucleotide of guanosine is cytosine. Nucleotides can also be non-naturally occurring or modified bases. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. The pairing of purine containing nucleotide (e.g., A or G) with a pyrimidine containing nucleotide (e.g., T or C) are considered complements. The A-T and C-G pairings function to form double or triple hydrogen bonds between the amine and carbonyl groups on the complementary bases. Complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In one embodiment, two sequences are complementary when they are completely complementary, having 100% complementarity. In one embodiment, two sequences are complementary when they are functionally complementary, i.e., successfully anneal under appropriate conditions and having at least 80%-99% or at least 70% to 95% base pairing.

### EXAMPLES

### Example 1—Materials

### Materials Examples 1 to 9

Recombinant proteins. A synthetic gene encoding the SARS-CoV-2 (Hu-1 isolate) S ectodomain, corresponding to the S2P protein described by Wrapp et al., 2020 was obtained from GeneART-ThermoFisher Scientific. The gene encodes S residues 16-1208, the furin cleavage site mutation, R⁶⁸²RAR->G⁶⁸²SAS, and a di-Pro substitution at positions 986 and 987. The C-terminus of S2P was appended with foldon (YIPEAPRDGQAYVRKDGEWVLLSTFL) (SEQ ID NO:20), octa-His and avitag (GLNDIFEAQKIEWHE) sequences (referred to collectively as FHA or the FHA tag SEQ ID NO:21), each separated by GSGS linkers. The synthetic S2P gene was ligated downstream of a DNA sequence encoding the tissue plasminogen activator leader via NheI, within pcDNA3 (Invitrogen). Mutations were introduced into S2P expression vectors using synthetic genes encoding mutated S2P subfragments produced by GeneART-ThermoFisher Scientific. Synthetic genes encoding the S1 sub-unit (amino acids 16-682) and the receptor binding domain (RBD; amino acids 332-532) were obtained by GeneART-ThermoFisher Scientific and ligated to the tissue plasminogen activator leader via NheI in pcDNA3. Both proteins encode a C-terminal hexa-His tag and Avitag sequence. hACE2-Fc is a recombinant fusion protein comprising amino acids 19-615 of the human ACE2 ectodomain linked to the Fc domain of human IgG1 via a GS linker. A synthetic gene encoding hACE2-Fc was obtained from GeneART-ThermoFisher Scientific and ligated downstream of the tissue plasminogen activator leader via NheI in pcDNA3. The DNA sequences of S and hACE2 clones were verified by fluorescent Sanger sequencing (BigDye, ABI).

Expression and purification of recombinant proteins. S2P expression vectors were transfected into 293Freestyle cells using 293fectin as recommended by the manufacturer (ThermoFisher Scientific). The cells were cultured for 5 days at 34° C. after which the transfection supernatants were cleared of cells by centrifugation and filtration through 0.45 μm nitrocellulose filters. S2P proteins were then purified by divalent cation affinity chromatography using TALON resin (Merck) followed by size exclusion chromatography (SEC) using a Superose 6 Increase 10/300 column linked to an AKTApure instrument (Cytiva). S1 and RBD proteins were produced by transfection of Expi293 cells with the appropriate expression vectors using Expifectamine according to the manufacturer's instructions (ThermoFisher Scientific). After 4 days of culture at 34° C., the transfection supernatants were cleared of cells by centrifugation and filtration through 0.45 μm nitrocellulose filters. The S1 and RBS proteins were purified by divalent cation affinity chromatography using TALON resin (Merck) followed by SEC using a Superdex 200 16/600 column linked to an AKTApure instrument (Cytiva). hACE2-Fc was produced in Expi293 cells as for S1 and RBD and purified from the clarified culture supernatant using Protein G-Agarose (Genscript). The hACE2-Fc was further purified by SEC on a Superdex 200 16/600 column linked to an AKTApure instrument (Cytiva). All proteins were concentrated using Amicon centrifugal filter units. The protein solutions were filter-sterilized using 0.45 μm nitrocellulose filters and protein aliquots stored at −80° C. Protein purity was assessed by SDS-PAGE and SEC.

Recombinant monoclonal antibodies (mAbs). pCDNA3-based IgG1 heavy and light chain expression vectors (Center et al. 2020) containing the variable regions of SARS-CoV-2 directed mAbs CR3022 (Muelen, PloS Med 2006), CB6 (Shi, Nature 2020), H4 and B38 (Wu et al., 2020), 2-51 (Liu, et al., 2020), COVA2-15, COVA2-33, COVA1-25, COVA1-22, COVA2-14 (Brouwer et al., 2020) were produced in-house using synthetic gene fragments encoding the mAb heavy and light chain variable regions produced by GeneART-ThermoFisher Scientific. The mAbs were produced by transfection of Expi293 cells with equal amounts of matched heavy and light chain vectors using Expifectamine according to the manufacturer's instructions (ThermoFisher Scientific). After 5 days of culture at 37° C., the transfection supernatants were cleared of cells by centrifugation and filtration through 0.45 μm nitrocellulose filters. The IgG was purified by affinity chromatography using Protein G-agarose (Genscript) and exchanged into PBS. The antibodies were concentrated using Amicon centrifugal filter units. IgG solutions were filter-sterilized using 0.45 μm nitrocellulose filters and aliquots stored at −80° C.

Differential scanning fluorimetry. Differential scanning fluorimetry was used to assess protein thermostability (Niesen Nature Protocols 2007). 10 μg of protein was diluted into 25 μL with 5× concentration SYPRO Orange Protein Gel Stain (Sigma Aldrich) in duplicate. The samples were then heated in an Mx3005P qPCR System in 0.5° C. increments from 25° C. to 95° C. for 1 minute per increment. 3 measurements of fluorescence were taken at the end of each increment. Excitation was at 492 nm, and emission at 610 nm. The melting temperature was determined to be the minimum of the negative first derivative of the melting curve.

Biolayer interferometry. BLI-based measurements (BLI) were determined using an OctetRED System (ForteBio, Fremont CA). Antibodies were diluted in kinetic buffer to 10 μg/ml and immobilized onto anti-human IgG Fc capture biosensors (AHC, ForteBio). Kinetics assays were carried out at 30° C. using standard kinetics acquisition rate settings (5.0 Hz, averaging by 20) at a sample plate shake speed of 1,000 rpm. The kinetic experiments included five steps: (a) baseline (180 s); (b) antibody loading (300 s); (c) second baseline (180 s); (d) association of antigen (300 s), and (e) dissociation of antigen (300 s). Fitting curves were constructed using ForteBio Data Analysis 10.0 software using a 1:1 binding model, and double reference subtraction was used for correction.

Immunizations. Guinea pigs (outbred tricolor) that were matched for gender, weight, and age were immunized subcutaneously with 30 μg of S2P proteins in PBS in a 1:1 (v/v) mix with AddaVax adjuvant (InvivoGen, San Diego, CA) at weeks 0, 4 and 14. A negative control group was immunized as above with a 1:1 (v/v) mix of PBS and adjuvant. Blood was collected at 2 weeks after the 2^{nd} dose via the saphenous vein, and at 2 weeks after the 3^{rd} dose by terminal cardiac puncture and allowed to clot for serum preparation. Sera were stored at −80° C., with heat inactivation at 56° C. for 30 min prior to use in immunological assays. Animals were housed and all procedures were performed at the Preclinical, Imaging, and Research Laboratories, South Australian Health and Medical Research Institute (Gilles Plains, Australia). All animal experiments were performed in accordance with the eighth edition of the Australian Code for the Care and Use of Animals for Scientific Purposes and were approved by the SAHMRI Animal Ethics Committee, project number SAM-20-030.

ELISA. Nunc maxisorp 96 well plates were coated with S2P, S1 and RBD protein solutions (2 μg/ml, PBS) at 4° C. overnight. The plates were washed with PBS and blocked with BSA (10 μg/ml, PBS) at room temperature for 1 h. The plates were again washed and then incubated with serially diluted serum samples for 2-4 h at room temperature. Antibody binding was detected using horseradish peroxidase-labelled rabbit anti-guinea pig antibody (Dako, Glostrup, Denmark) and 3,3′,5,5′ 5,5′-dithiobis-(2-nitrobenzoic acid)tetramethylbenzidine dihydrochloride (TMB). Antibody binding to different antigens was compared by fitting curves with nonlinear regression using Prism version 9 software, and titers were obtained by interpolation of optical density (OD) values 10-fold above that of background, as defined by binding to BSA.

Pseudotyped virus production. S-pseudotyped HIV luciferase reporter viruses were prepared according to the method of Jackson et al., 2020. Plasmids for the production of S-HIV pseudoparticles were a kind gift of Professor Doria-Rose, NIH Vaccine Research Centre, and include the WH-Human1_EPI_402119 expression plasmid bearing codon-optimized full-length S (Genbank #MN908947.3), the packaging plasmid pCMVAR8.2 and luciferase reporter plasmid pHR′ CMV Luc (Naldini PNAS 1996; 93:11382), and a TMPRSS2 plasmid (Bottcher JVI 2006, 80:9869). The 4 plasmids were co-transfected into HEK293T cells and after 18 h of incubation, the medium was replaced with fresh Dulbecco's modification of minimal essential medium containing 10% fetal bovine serum (DMF10) and cultured for a further 3 days. Clarified supernatants containing retroviral pseudotyped viruses were filtered through 0.45 μm membrane filters. Mutations observed in variants of concern were introduced into the S open reading frame in WH-Human1_EPI_402119 by overlap-extension polymerase chain reaction.

Neutralizing assay. Neutralizations were conducted according to the method of Jackson et al., 2020. Heat inactivated sera (56° C. for 30 minutes) were serially diluted in DMF10 and each dilution mixed with an equal volume of S-pseudotyped HIV luciferase reporter viruses and incubated for 1 h at 37° C. in triplicate. Virus-serum mixtures were added to 293T-ACE2 cells monolayers attached to poly-L-lysine coated 96 well plates the day prior at 5,000 cells/well, and incubated for 2 h at 37° C. before addition of an equal volume of DMF10. After 3 days, tissue culture fluid was removed, monolayers were washed once with PBS and lysed with cell culture lysis reagent (Promega) and luciferase measured using luciferase substrate (Promega) in a Clariostar plate reader (BMG LabTechnologies). The mean percentage entry was calculated as (RLU plasma+virus)/(RLU medium+virus)*100. The percentage entry was plotted against the reciprocal dilution of plasma in Prism v8.3.1 and curves fitted with a one-site specific binding Hill plot. The reciprocal dilution of plasma required to prevent 50% virus entry was calculated from the non-linear regression line (ID50). The lowest amount of neutralizing antibody detectable is a titre of 200. All samples that did not reach 50% neutralization were assigned an arbitrary value of 100.

Serum-mAb cross-competition ELISA. Biotinylated S2P proteins were produced in 293Expi-BirA cells (293Expi cells that stably express BirA) and purified as described above for S2P. For competition ELISAs, Nunc maxisorp 96-well plates were coated with streptavidin (Sigma) (5 μg/ml in 50 mM carbonate buffer) at 4° C., overnight after which they were blocked with BSA (10 mg/ml in PBS) at room temperature for 1 h. After 2 washes, the plates were incubated with biotinylated S2P trimers (2 μg/ml in 5 mg/ml BSA/PBS containing 0.05% Tween 20) at room temperature for 1 h. Serially diluted vaccinal sera were mixed with sub-saturating amounts of hACE2-Fc and anti-SARS-CoV-2 S mAbs and incubated with the streptavidin-biotinylated S2P coated plates for a further 2 h at room temperature. mAb binding was detected using horseradish peroxidase-labelled goat anti-human IgG F(ab′)2 (Thermofisher-Scientific), or horseradish peroxidase-labelled anti-human IgA, IgG, IgM (Dako, Glostrup, Denmark) for hACE2-Fc. The substrate was TMB. Color reactions were measured with a Multiskan Ascent plate reader (Thermo Electron, Waltham, MA). Antibody binding to different antigens was compared by fitting curves with nonlinear regression using Prism version 9 software, and ID50s obtained by interpolation.

Statistical methods. Data were statistically compared using the non-parametric Kruskal-Wallis test with Dunn's multiple comparisons in Prism 9. For neutralization assays, a Friedman test was used to compare paired samples.

### Materials Examples 10-11

Recombinant proteins. Synthetic genes encoding the SARS-CoV-2 Hu-1, Delta (B.1.617.2) and Omicron BA.1 (B.1.1.529) receptor binding domain (S residues 332-532, according to the Hu-1 numbering system) were obtained from GeneART-ThermoFisher Scientific. The C-termini of the RBDs were appended with the GGSGS-octaHis-GSGS-avitag (GLNDIFEAQKIEWHE) sequence. GSGSGS=SEQ ID NO:22 GSGS=SEQ ID NO: 23. The synthetic RBD-His8-avitag genes were ligated downstream of a DNA sequence encoding the tissue plasminogen activator leader via NheI, within pcDNA3 (Invitrogen). Synthetic genes encoding the SARS-CoV-2 Delta and Omicron BA.1 isolate S ectodomain, corresponding to the S2P protein described by Wrapp et al., 2020 were obtained from GeneART-Thermo Fisher Scientific. The gene encodes S residues 16-1208 (according to the Hu-1 numbering system), the furin cleavage site mutations, R681RRAR->P681GSAS for Delta and H681RRAR->P681GSAS for Omicron BA.1, and the di-Pro ‘2P’ substitution at positions 986 and 987. The C-terminus of S2P was appended with foldon (YIPEAPRDGQAYVRKDGEWVLLSTFL: SEQ ID NO: 20), octa-His and avitag (GLNDIFEAQKIEWHE; SEQ ID NO: 21) sequences (referred to collectively as FHA or the FHA tag), each separated by GSGS linkers. A synthetic gene encoding S residues 16-1208 of the Hu-1 isolate, the furin cleavage site mutation, R682RAR->G682SAS, and a di-Pro substitution at positions 986 and 987 was appended at the 3′ end with a synthetic DNA sequence encoding a GSGS linker and a hexa-His tag to give S2P-1208.H6. The synthetic S2P genes were ligated downstream of a DNA sequence encoding the tissue plasminogen activator leader via NheI, within pcDNA3 (Invitrogen). Mutations were introduced into S2P expression vectors using synthetic genes encoding mutated S2P subfragments produced by GeneART-ThermoFisher Scientific.

Expression and purification of recombinant proteins. Biotinylated RBD and S2P-FHA proteins were produced by transfection of Expi293-BirA cells with the appropriate expression vectors using Expifectamine according to the manufacturer's instructions (ThermoFisher Scientific). After 4 days of culture at 34° C., the transfection supernatants were cleared of cells by centrifugation and filtration through 0.45 m nitrocellulose filters. The RBD and S2P-FHA proteins were purified by divalent cation affinity chromatography using TALON resin (Merck) followed by size exclusion chromatography using a Superdex 200 16/600 or Superose 6 column, respectively, linked to an AKTApure instrument (Cytiva). Non-biotinylated S2P-FHA and S2P-1208.H6 proteins were produced by transfection of either FreeStyle™ 293 cells (293Freestyle) cells or Expi293F™ (Expi293F) cells with the appropriate expression vectors using 293Fectin or Expifectamine, respectively, according to the manufacturer's instructions (ThermoFisher Scientific). The cells were cultured for 5 days at 34° C. after which the transfection supernatants were cleared of cells by centrifugation and filtration through 0.45 μm nitrocellulose filters. S2P proteins were then purified by divalent cation affinity chromatography using TALON resin (Merck) followed by size exclusion chromatography using a Superose 6 Increase 10/300 column linked to an AKTApure instrument (Cytiva). When required, the TALON affinity chromatography step was replaced with a chromatographic step using a HiTrap Chelating HP immobilized metal affinity column (Cytiva) linked to an AKTApure instrument (Cytiva). In this case, the S2P protein was eluted using an imidazole concentration gradient.

Recombinant monoclonal antibodies (mAbs). pCDNA3-based IgG1 heavy and light chain expression vectors (Center et al. 2020) containing the variable regions of SARS-CoV-2 directed mAbs COVOX222 (Dejnirattisai et al. 2021), S2H97 and S2E12 (Starr et al. 2021), CV3-25 (Jennewein et al. 2021) and the control HCV-directed mAb, HC33.1 (Center et al. 2020) were produced in-house using synthetic gene fragments encoding the mAb heavy and light chain variable regions produced by GeneART-ThermoFisher Scientific. The mAbs were produced by transfection of Expi293F cells with equal amounts of matched heavy and light chain vectors using Expifectamine according to the manufacturer's instructions (ThermoFisher Scientific). After 5 days of culture at 37° C., the transfection supernatants were cleared of cells by centrifugation and filtration through 0.45 μm nitrocellulose filters. The IgG was purified by affinity chromatography using Protein G-agarose (Genscript) and exchanged into PBS. The antibodies were concentrated using Amicon centrifugal filter units. IgG solutions were filter-sterilized using 0.45 μm nitrocellulose filters and aliquots stored at −80° C.

Avidin Capture ELISA. Nunc maxisorp 96-well plates were coated with avidin (Rockland) (5 μg/ml in 50 mM carbonate buffer) at 4° C., overnight after which they were blocked with BSA (10 mg/ml in PBS) at room temperature for 1 h. After 4 washes, the plates were incubated with biotinylated RBD proteins (2 μg/ml in 5 mg/ml BSA/PBS containing 0.05% Tween 20) at room temperature for 1 h. Serially diluted vaccinal sera were incubated with the avidin-biotinylated RBD coated plates for a further 2 h at room temperature. Antibody binding was detected using horseradish peroxidase-labelled rabbit anti-guinea pig IgG (DAKO). The substrate was TMB (3,3′,5,5′-Tetramethylbenzidine). Color reactions were measured with a Multiskan Ascent plate reader (Thermo Electron, Waltham, MA). Antibody binding to different antigens was compared by fitting curves with nonlinear regression using Prism version 9 software, and endpoint titres determined as 5-times the background OD obtained in the absence of primary antibody.

Streptavidin Capture ELISA. Nunc maxisorp 96-well plates were coated with streptavidin (Sigma) (5 μg/ml in 50 mM carbonate buffer) at 4° C., overnight after which they were blocked with BSA (10 mg/ml in PBS) at room temperature for 1 h. After 2 washes, the plates were incubated with biotinylated S2P-FHA trimers (2 μg/ml in 5 mg/ml BSA/PBS containing 0.05% Tween 20) at room temperature for 1 h. Serially diluted human mAbs were incubated with the streptavidin-biotinylated S2P-FHA coated plates for a further 2 h at room temperature. mAb binding was detected using horseradish peroxidase-labelled goat anti-human IgG F(ab′)2 (Thermofisher-Scientific). The substrate was TMB (3,3′,5,5′-Tetramethylbenzidine). Color reactions were measured with a Multiskan Ascent plate reader (Thermo Electron, Waltham, MA).

Live virus neutralization assays. The rapid high-content SARS-CoV-2 microneutralisation assay with HAT-24 cells (R-20 assay) platform developed by Aggarwal et al. 2021 was used to determine live virus neutralization ID₅₀ values of vaccinal sera. HAT-24 cells were trypsinised, resuspended in DMEM-5% fetal bovine serum medium with Hoechst-33342 live nuclear dye (Invitrogen, R37605) at 5% v/v, and seeded in 384-well plates (Corning, CLS3985) at 1.6×10⁴ cells/well. Guinea pig sera were serially diluted (2-fold) in DMEM-5% FBS and mixed in duplicate with an equal volume of SARS-CoV-2 virus solution at 2× the median lethal dose (2×LD50). After 1 hour of virus-serum incubation at 37° C., 40 μL were added to an equal volume of pre-plated cells. Cell plates were then incubated for 20 hours before direct imaging on an InCell Analyzer HS2500 high-content fluorescence microscopy system (Cytiva). Cellular nuclei counts were obtained with IN Carta automated image analysis software (Cytiva), and the percentage of virus neutralisation was calculated with the formula: % N=(D−(1−Q))×100/D, where “Q” is a well's nuclei count divided by the average count for uninfected controls (defined as having 100% neutralisation) and D=1−Q for the average count of positive infection controls (defined as having 0% neutralisation). The cut-off for determining the neutralisation endpoint titre of diluted serum samples was set to the last consecutive dilution reaching ≥50% neutralisation for the average of technical replicates.

Chemical crosslinking. Thyroglobulin (0.5 mg/ml in PBS, Cytiva) was chemically crosslinked with 1 mM bis(sulfosuccinimidyl)suberate (Thermo Fisher) in PBS for 1 h on ice. The reaction was quenched with 30 mM glycine in PBS, pH 7.2 for 30 min on ice.

Construction of S2P-1273 expression vectors. Synthetic genes encoding S residues 1-1273 of Hu-1, Delta and Omicron BA.1 variants were produced by GeneART-Thermo Fisher. The genes contained a KpnI restriction site followed by the TATCGCCACC (SEQ ID NO: 24) sequence at the 5′ end (prior to the ATG start codon) and an Xbal site (after the TAA stop codon) at the 3′ end. The synthetic genes encoded furin cleavage site mutations, R682RAR->G682SAS for Hu-1, R681RRAR->P681GSAS for Delta and H681RRAR->P681GSAS for Omicron BA.1, and the di-Pro ‘2P’ substitution at positions 986 and 987. The synthetic genes were cloned into the KpnI-EcoRV sites of the CMV promoter-driven expression vector pSHUTTLE (Agilent).

Western blotting of S2P-1273 glycoproteins expressed in 293T cells. 293T cells were transfected with the S2P-1273 expression vectors using FUGENE HD (Promega) according to the manufacturer's instructions. At 48 h post-transfection, the cells were washed with ice-cold PBS, centrifuged for 90 sec at 10,000 rpm and the pellets lysed in lysis buffer (1% Triton X100 in PBS containing 1 mM ethylenediamine tetraacetic acid) for 30 min on ice. The lysates were clarified by centrifugation at 10,000 rpm for 10 min at 4° C. and subjected to SDS-PAGE in the presence of 3% betamercaptoethanol. The proteins were transferred to nitrocellulose using the iBLOT2 system (Thermo Fisher), and the membranes blocked with 5% skim milk powder in PBS. The filters were probed with Rabbit anti-S1 polyclonal antibody (Sino Biological) and anti-rabbit IRDye800CW (Odyssey). The filters were then scanned in a LI-CORE imager.

Flow Cytometry. 293T cells were transfected with the S2P-1273 expression vectors using FUGENE 6 (Promega) according to the manufacturer's instructions. At 48 h post transfection, the attached cells were washed with PBS and then detached using versene solution. The cells were resuspended in 800 μl of FACS buffer (5% v/v fetal calf serum in PBS containing 2 mM ethylenediamine tetraacetic acid). The cells were added to u-bottom 96-well culture plates and incubated with 5 μg/ml of human mAbs in FACS buffer for 1 h at room temperature. The cells were washed twice in ice-cold FACS buffer by centrifugation at 400×g for 5 min. The cells were then incubated with AlexaFluor 647 goat anti-human (H+L) (Invitrogen) for 30 min at room temperature in the dark. The cells were washed twice in ice-cold FACS buffer by centrifugation at 400×g for 5 min. The cells were resuspended in 100 μl FACS buffer. Propidium iodide was added to a final concentration of 2.5 μg/ml before each flow cytometry run to enable exclusion of dead cells during analysis. The cells were applied to a Canto II flow cytometer immediately after addition of propidium iodide. Ten thousand events were captured for each antibody-S2P-1273 protein combination. FlowJo software was used for data analysis.

### Materials Example 12

Western blotting of S2P-1273 and S2P.omicron.BA.1-1273 glycoproteins expressed in 293T cells following treatment at various temperatures. 293T cells were transfected with the S2P-1273 and S2P.omicron-1273 expression vectors using FUGENE HD (Promega) according to the manufacturer's instructions. At 48 h post-transfection, the cells were washed with ice-cold PBS, centrifuged for 90 sec at 10,000 rpm and the pellets lysed in lysis buffer (1% Triton X100 in PBS containing 1 mM ethylenediamine tetraacetic acid) for 30 min on ice. The lysates were clarified by centrifugation at 10,000 rpm for 10 min at 4° C. The clarified lysates were split into equal volumes and adjusted to contain a final concentration of 1.2% (w/v) SDS and 0.25% (v/v) betamercaptoethanol. The samples were treated at various temperatures for 5 min and subjected to SDS-PAGE in 5% polyacrylamide gels. The proteins were transferred to nitrocellulose using the iBLOT2 system (Thermo Fisher), and the membranes blocked with 5% skim milk powder in PBS. The filters were probed with Rabbit anti-S1 polyclonal antibody (Sino Biological) and anti-rabbit IRDye800CW (Odyssey). The filters were then scanned in a LI-CORE imager.

Recombinant S2P.omicron-1208.H6 proteins. A synthetic gene encoding S residues 16-1208 (Hu-1 numbering system) of the Omicron BA.1 isolate, the furin cleavage site mutation, H681RRAR->P681GSAS, and a di-Pro substitution at positions 986 and 987 was appended at the 3′ end with a synthetic DNA sequence encoding a GSGS linker and a hexa-His tag to give S2P.omicron-1208.H6. The synthetic gene was obtained from GeneART ThermoFisher Scientific. S2P genes were ligated downstream of a DNA sequence encoding the tissue plasminogen activator leader via NheI, within pcDNA3 (Invitrogen). Mutations were introduced into S2P expression vectors using synthetic genes encoding mutated S2P subfragments produced by GeneART ThermoFisher Scientific.

Expression and purification of S2P.omicron-1208.H6 proteins. S2P.omicron-1208.H6 proteins were produced by transfection of Expi293F (Expi293F) cells with the appropriate expression vectors using Expifectamine according to the manufacturer's instructions (ThermoFisher Scientific). The cells were cultured for 7 days at 34° C. after which the transfection supernatants were cleared of cells by centrifugation and filtration through 0.45 μm nitrocellulose filters. S2P.omicron-1208 proteins were then purified by divalent cation affinity chromatography using a HiTrap Chelating HP immobilized metal affinity column (Cytiva) linked to an AKTApure instrument (Cytiva). The S2P protein was eluted using an imidazole concentration gradient. S2P.omicron-1208 trimers were purified by size exclusion chromatography using a Superdex 200 16/600 or Superose 6 column linked to an AKTApure instrument (Cytiva).

### Example 2—Characterization of S2P

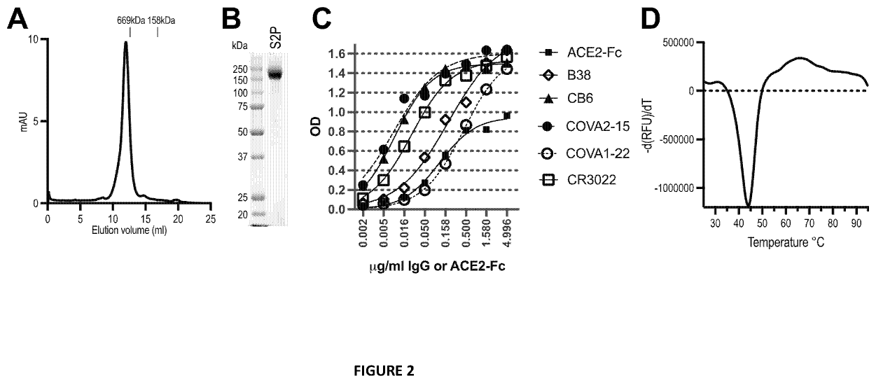

A CMV promoter driven expression vector was used to produce a soluble form of the S glycoprotein known as S2P (Wrapp et al., 2020), which comprises S residues 16-1208, a furin cleavage site mutation, R⁶⁸²RAR->G⁶⁸²SAS, a di-Pro substitution at positions 986 and 987. Foldon, octa-His and avitag sequences were added to the C-terminus to give S2P-FHA. Following partial purification by divalent cation affinity chromatography, SEC of the S2P-FHA protein revealed a major peak coeluting with thyroglobulin (669 kDa) that was collected as a homogenous protein as indicated by SEC and SDS-PAGE (FIGS. 2A and B). A biotinylated form of purified S2P-FHA exhibited binding activity with hACE2-Fc, (human ACE2 residues 19-615 linked to the Fc domain of human IgG1) and various human mAbs in ELISA (FIG. 2 C). A thermofluor assay indicated that the S2P protein possessed relatively low thermal stability, exhibiting a melting temperature of 43.6° C. (FIG. 2 D).

### Example 3—Effects of Ala Cavity Substitutions on Thermostability

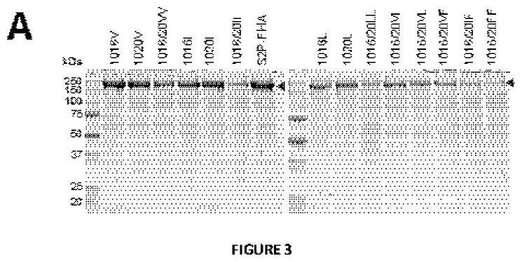

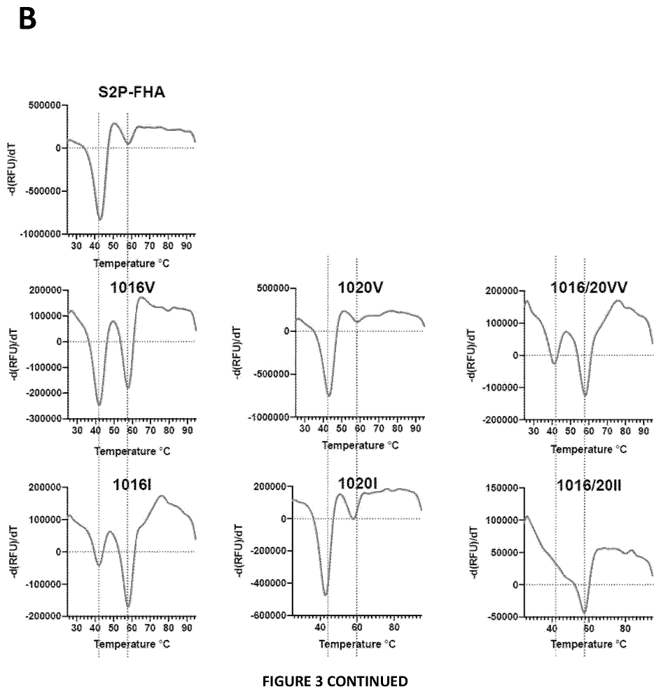

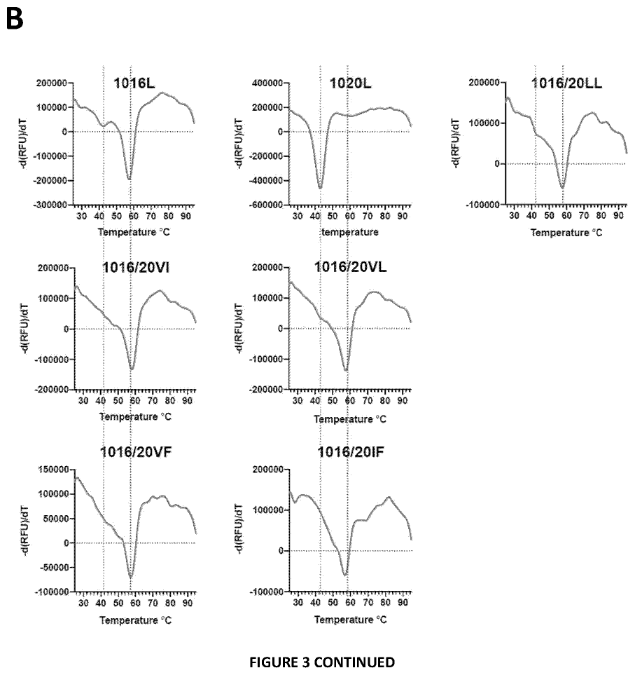

The effect of replacing Ala1016/1020 with bulkier hydrophobic residues (Val, Leu, Ile, Phe) was assessed on the stability and antigenic structure of the SARS-CoV-2 S trimer. (2 examples are shown in FIG. 1D). Alanine¹⁰¹⁶ and A¹⁰²⁰ were singly and doubly substituted with Val, Ile, Leu and Phe in S2P-FHA and the 293F-expressed glycoproteins partially purified by divalent cation affinity chromatography. SDS-PAGE indicated a range of yields: S2P-FHA, 1016V, 1020V, 1020I>1016/20VV, 1016I, 1020I, 1016L, 1020L, 1016/20VI>1016/20II, 1016/20LL, 1016/20VL, 1016/20VF, 1016/20IF>1016/20FF (FIG. 3A). A thermofluor assay was used to examine the thermostability of S2P-FHA and the mutants. S2P-FHA comprised a major species with a melting temperature of 43.6° C. and a more stable minor species with a melting temperature of 58° C. (FIG. 3 B). The substitution of hydrophobic residues into position 1016 increased the proportion of the 58° C. species with A1016L being the most stabilizing mutation. By contrast, the 43.6° C. species remained the predominant form with substitutions of A¹⁰²⁰. Double substitutions were all associated with the stable form, except for 1016/20VV.

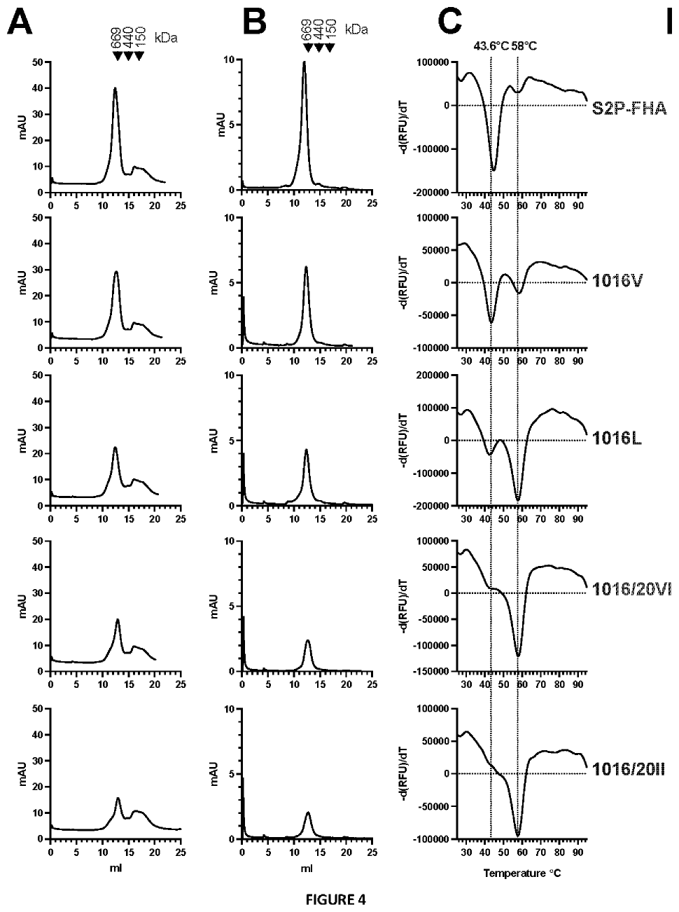

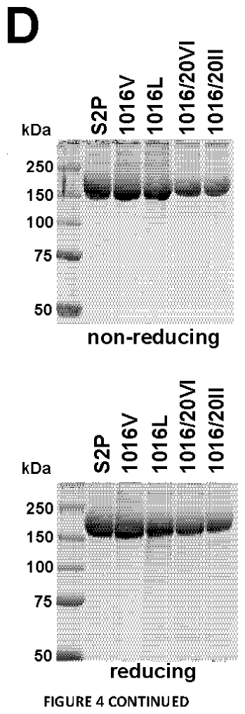

Representative mutants were purified to homogeneity and re-analysed in the thermofluor assay (FIG. 4A-C). The thermofluor data obtained with partially pure proteins were largely recapitulated with the purified trimers. Increasing proportions of the 58° C. form relative to the 43.6° C. form was observed for the mutants as follows: 1016/20VI=1016/20II>1016L>1016V>S2P-FHA (FIG. 4 C). Interestingly, this hierarchy of stability was inversely correlated to trimer yield (FIG. 4 B). FIG. 4 D confirms the purity of the purified trimers as assessed by SDS-PAGE under non-reducing and reducing conditions.

### Example 4—Antigenic Properties of Ala Cavity Mutants

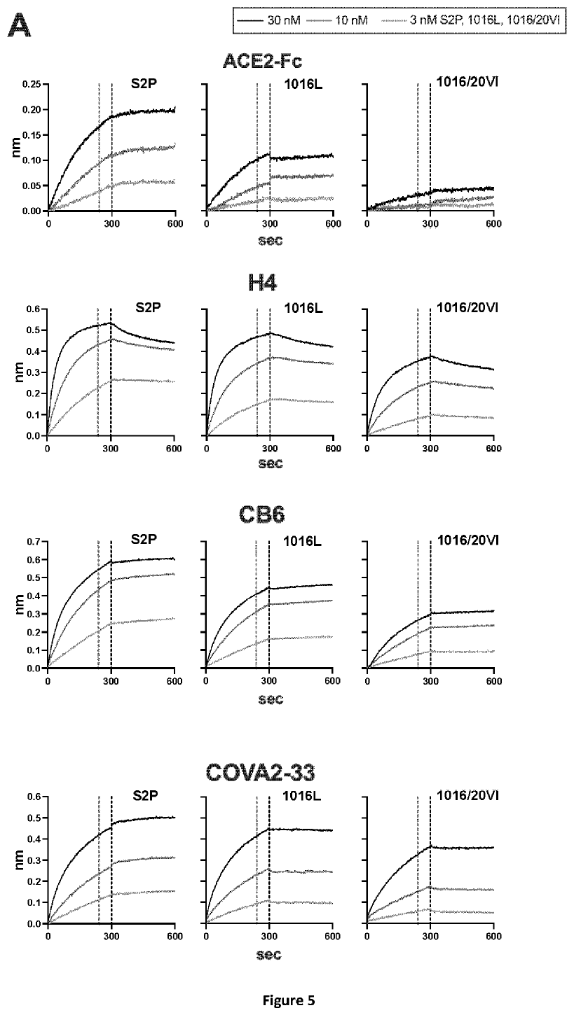

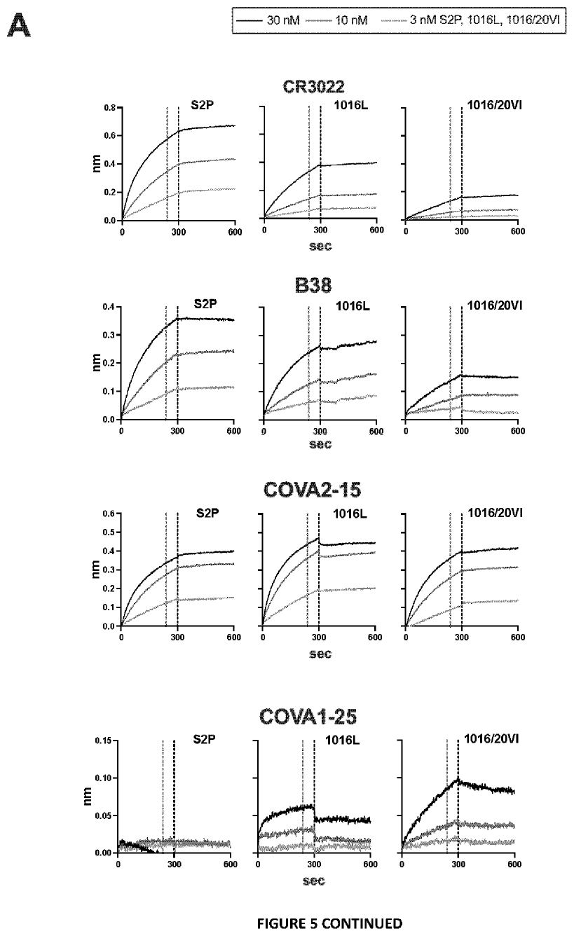

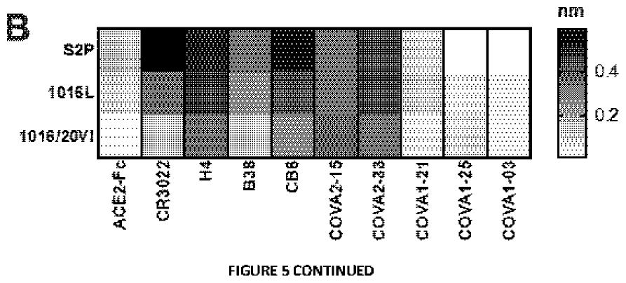

The 1016L and 1016/20VI mutants exhibited favourable thermal properties and could be purified at reasonable yields. Their antigenic properties were therefore compared with those of S2P by examining their binding to hACE2-Fc and recombinant human anti-S monoclonal antibodies (mAbs) in biolayer interferometry (BLI). hACE2-Fc and the mAbs were attached to anti-human IgG Fc capture (AHC) biosensors while the S2P proteins were in the analyte phase. Measurable off rates were not evident for the majority of S ligands, presumably due to avidity effects, precluding obtaining KD values (FIG. 5A). A comparison of sensograms indicated successive decreases in overall binding ability for 1016L and 1016/20VI relative to S2P-FHA for hACE2-Fc, and mAbs that interfere with the S-ACE2 interaction such as H4, B38 and CB6. This binding pattern was also observed for CR3022, a mAb that is directed to an epitope at the base of the RBD that is exposed when an RBD is in the “up” orientation. By contrast, successive increases in binding were observed for 1016L and 1016/20VI, respectively, to COVA1-25 that is directed to an epitope in S that is external to the RBD (FIG. 5A). The data are summarised in a heat map (FIG. 5B) showing the change in wavelength observed after 240 s of association with 30 nM of analyte. These data suggest that 1016L and 1016/20VI promote the RBD-down conformation in S2P-FHA trimers, while exposing an epitope within S but outside the RBD.

### Example 5—Immunogenicity of Ala Cavity Mutants

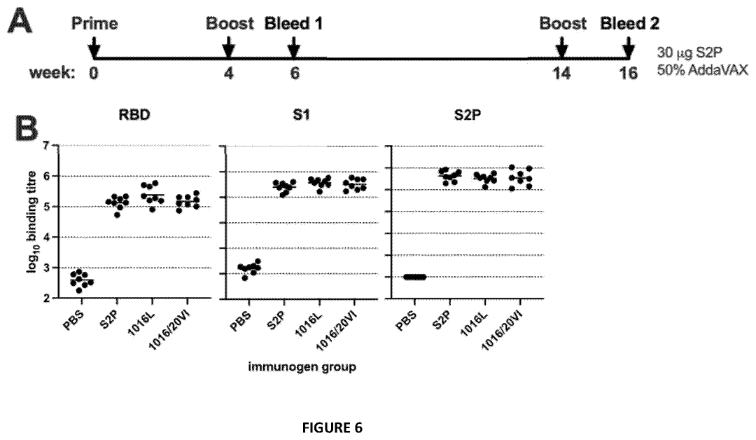

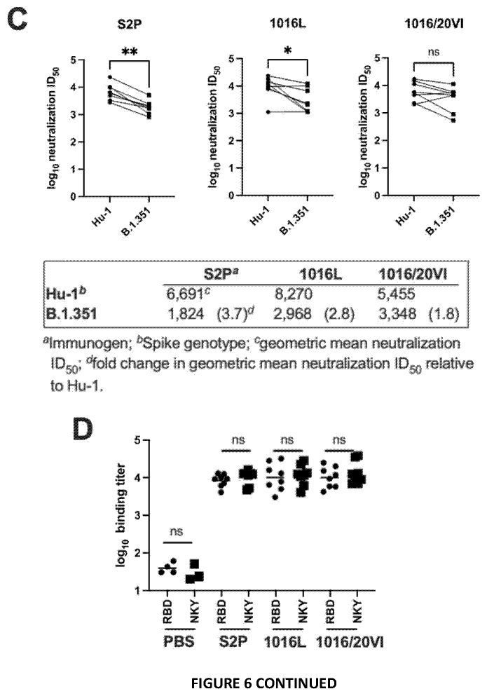

Guinea pigs were used to examine whether the Ala cavity mutations can affect the magnitude and specificity of antibody responses to S2P-FHA trimers. Outbred guinea pigs were immunized with 30 μg of S2P-FHA, 1016L and 1016/20VI in Addavax adjuvant at weeks 0, 4 and 14 and bleeds performed at weeks 6 and 16 (FIG. 6A). Antibody binding titers to RBD, S1 and S2P-FHA (Hu-1 sequence) proteins were determined for week 16 sera in ELISA. No significant differences were observed between immunogen groups with geometric mean binding titers were in the range of 1.3×10⁵-9.5×10⁵ (FIG. 6 B).

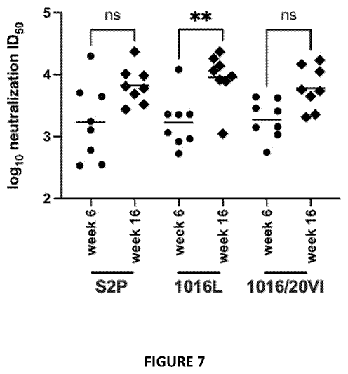

The neutralizing activity in vaccinal sera was determined using S-pseudotyped HIV luciferase reporter viruses and 293-ACE2 target cells as described by Jackson et al., 2020. A comparison of week-6 and week-16 sera (bleeds 2 weeks taken after the 1st and 2nd boosts, respectively) using pseudotypes containing the S2P-FHA immunogen-matched S glycoprotein (Hu-1) isolate indicated potent neutralizing activity in S2P-FHA-, 1016L- and 1016/20VI-immune sera with mean ID₅₀s ranging from 1,700-1,900 for week-6 sera and 6,000-9,100 for week-16 sera. These data equate to ˜3-5.4-fold increases in mean neutralization ID₅₀ following the 2^{nd} boost, although statistical significance was not reached for S2P-FHA and 1016/20VI-immune sera (FIG. 7). The neutralizing activity against pseudotypes containing (Jackson, 2020 #20) the beta (B.1.351) VOC S protein was next assessed. The mean neutralization potencies of S2P-FHA- and 1016L-immune sera were reduced by 3.7 and 2.8-fold, respectively, against beta S pseudotypes relative to the vaccine strain (Hu-1) whereas the small 1.8-fold reduction observed for 1016/20VI sera was not statistically significant. These data suggest that the 1016/20VI immunogen elicits NAb specificities that are not affected by the beta variant mutations. An ELISA was used to compare the binding of sera to the Hu-1 and beta variant RBDs, the latter containing the K417N, E484K, and N501Y (“NKY”) mutations (FIG. 6 C). No significant differences in binding titers were observed for the 2 antigens indicating that the decreased beta variant neutralization by S2P-FHA and 1016L sera cannot simply be explained by sensitivity to the NKY mutations in the RBD.

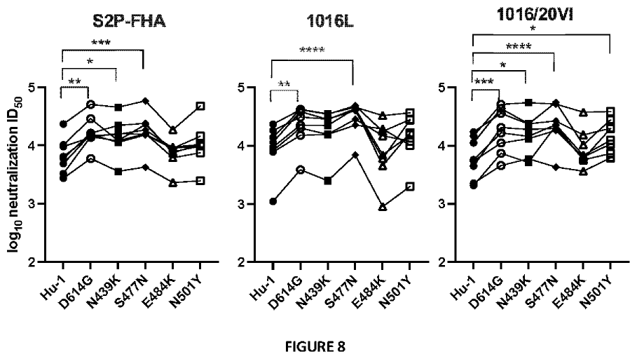

The neutralization activity of vaccinal sera against S pseudotypes bearing individual mutations observed in key variants of concern (VOCs and VOIs), including N439K, S477N, E484K and N501Y was next assessed. These mutations were combined with D614G, which is present in the vast majority of pandemic isolates, The sera exhibited an overall increase in neutralization potency against D614G, which was largely retained with the addition of N439K, S477N, and N501Y for 1016/20VI (FIG. 8). Interestingly, decreases in neutralization potency against the mutants were not evident when compared with Hu-1 S pseudotypes. Thus, the reductions in neutralization potencies for beta variant pseudotypes observed with S2P-FHA and 1016L sera could not be attributed to individual component mutations present in this variant.

### Example 6—Specificity of Antibody Responses

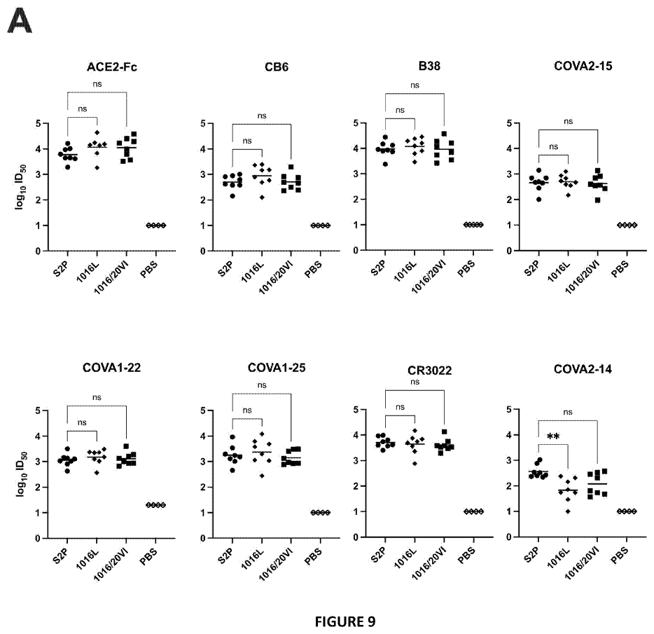

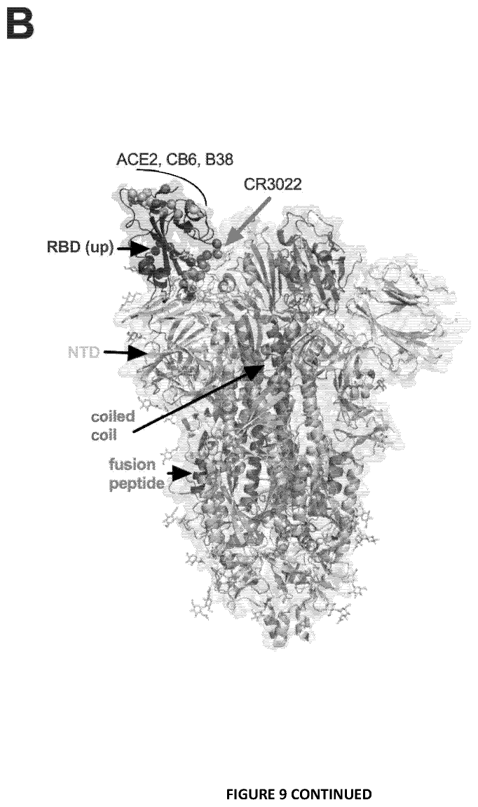

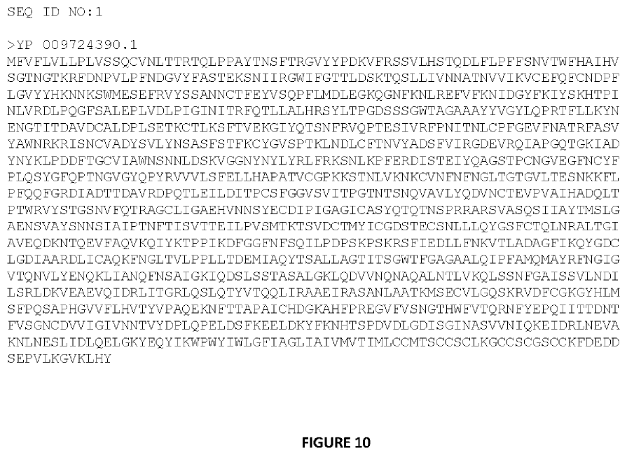

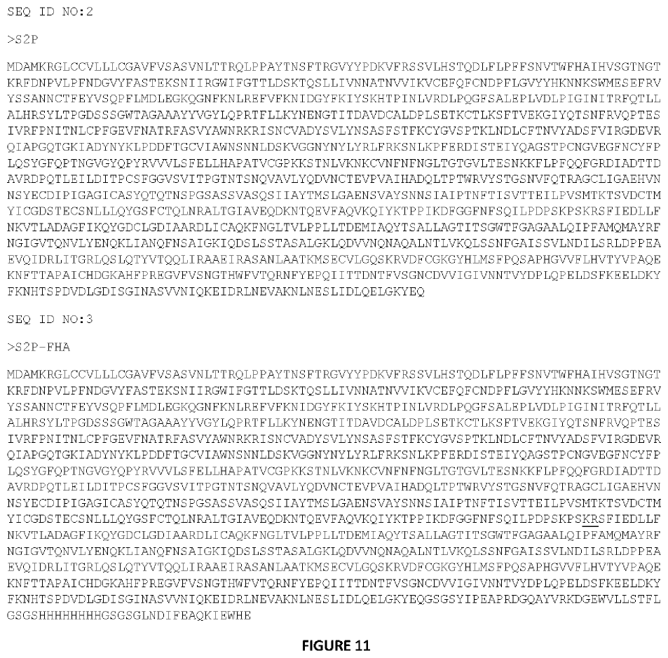

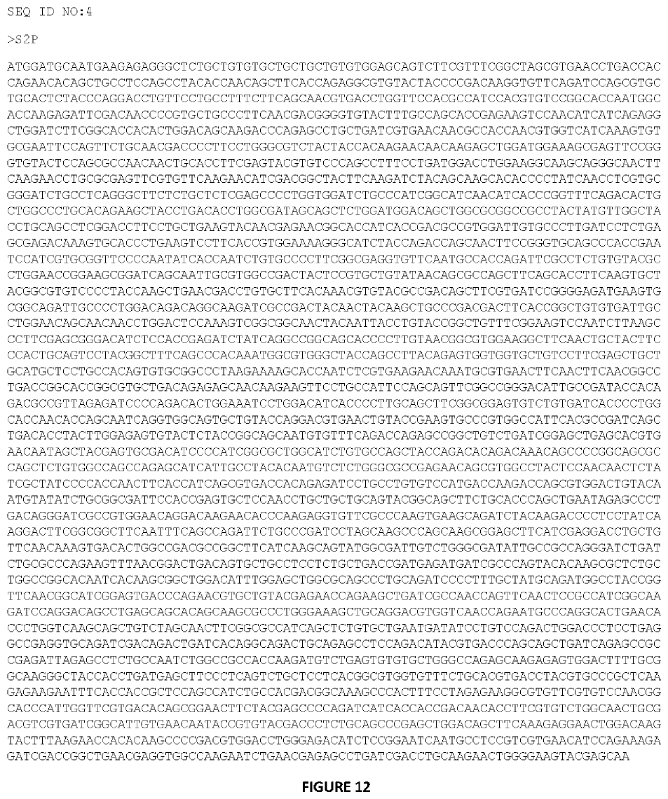

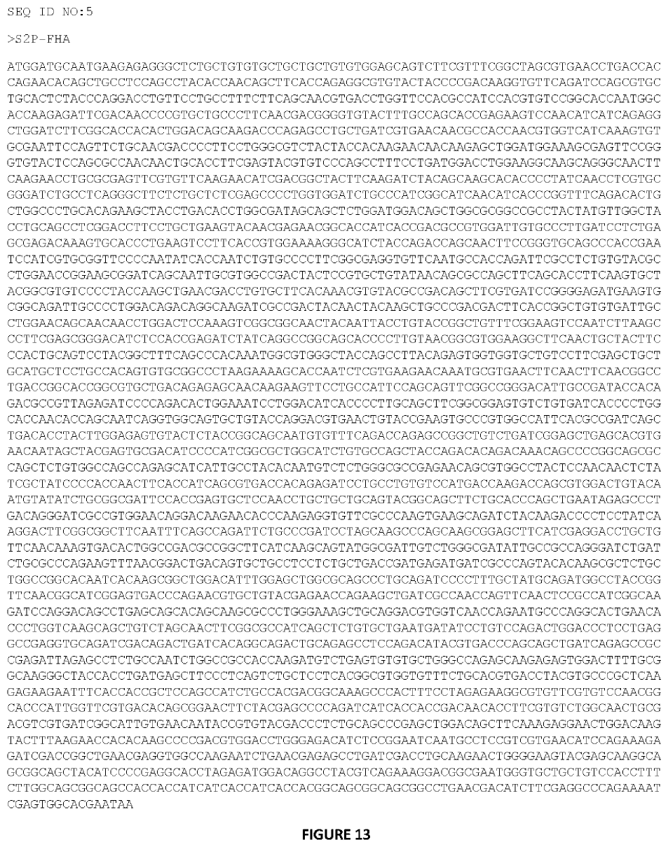

A serum-monoclonal antibody cross-competition assay was employed to gain an understanding of the specificity of antibody responses in the vaccinated animals. Biotinylated S2P-FHA captured onto streptavidin coated ELISA plates was incubated with a mixture comprising a sub-saturating amount of human monoclonal antibody and a dilution series of sera. The assay was developed with anti-human F(ab′)2-HRP and TMB. The data (FIGS. 9A and B) show that S2P-FHA, S2P.1016L-FHA and S2P.1016/20VI-FHA had largely equivalent abilities to elicit antibody specificities able to block S2P-FHA binding by ACE2-Fc and NAbs directed to the ACE2-binding site (CB6, B38 and COVA2-15), a NAb directed to the NTD (COVA1-22) and COVA1-25, which binds a non-RBD, non-NTD epitope in S. Sera from the 3 immunogen groups also exhibited blocking activities against CR3022, a non-neutralizing antibody directed to the base of the RBD which is obscured in the RBD down conformation. The data indicate that the 3 immunogens have similar abilities to elicit neutralizing antibodies to the ACE2-binding site, the NTD and an undefined epitope in S outside the RBD and NTD. These data also indicate that the 3 immunogens have similar abilities to induce antibodies directed to epitopes that overlap with the ACE2bs despite the finding in BLI experiments that this site appears to be less exposed in 1016L and 1016L/20VI.

### Example 7—Preclinical Evaluation

Preclinical evaluation of the stabilized Spike booster mRNA vaccine will be conducted in guinea pigs. Three groups of 10 guinea pigs per group will initially obtain 2 doses of mRNA encoding the ancestral Spike 3 weeks apart to mimic an mRNA-Spike vaccine regimen. Alternatively, 3 groups of 10 guinea pigs per group will receive 2 doses of adenovirus 5 encoding ancestral Spike 12 weeks apart to mimic the AstraZeneca vaccine regimen. The guinea pigs will be boosted with mRNA encoding: 1) ancestral Spike, 2) the novel stabilized Spike, or, 3) placebo. Serum neutralization and Spike protein binding (RBD and Spike trimer) will be determined at 14 and 28 days post-boosting. Serum neutralization assays will be conducted with the Spike-HIV luciferase reporter system (Jackson et al., 2020) and will include Spike proteins derived from VOCs. Spike binding will be determined in ELISA; Spike proteins derived from VOCs will be included in the analysis.

### Example 8—Phase I Safety and Immunogenicity Trial of the Universal Booster mRNA Vaccine

A Phase I clinical trial will be conducted to evaluate the safety and efficacy of the booster vaccine candidates (mRNA and adenovirus-vector). Each vaccine candidate will be tested through a randomized, double blind, placebo controlled clinical trial. Trial participants will be aged between 18 and 85 years who have already received the complete course of the current two-dose Pfizer (mRNA vaccine) or AstraZeneca (adenovirus vector vaccines) at least 6 months prior to the point of recruitment. Participants in the mRNA-stabilized spike booster trial will be assigned to 4 groups: Placebo, and 30 μg of mRNA-stabilized Spike vaccine (dose based on the current BioNTech-Pfizer vaccination regimen). The aim is to have 10 people in the placebo groups and at least 20 people in the booster vaccine groups (at least 60 people in total). The primary endpoints will be: 1) solicited local reactions, systemic events, and use of antipyretic or pain medication within 7 days after receipt of booster vaccine or placebo; 2) unsolicited adverse events and serious adverse events assessed through 6 months after receipt of the booster vaccine; 3) clinical laboratory abnormalities assessed 1 day and 7 days after receipt of booster vaccine; 4) grading shifts in laboratory assessments between baseline and 1 day and 7 days after the vaccine dose. Protocol-specified safety stopping rules will be in effect for all the participants. The secondary endpoints will be serum neutralization and Spike protein binding (RBD and Spike trimer) determined at 7, 14, 28, 180 and 365 days post-boosting. Serum neutralization assays will be conducted with the Spike-HIV luciferase reporter system (Jackson et al., 2020) and will include Spike proteins derived from VOCs. Spike binding will be determined in ELISA; Spike proteins derived from VOCs will be included in the analysis.

### Example 9—S2P-FHA has Enhanced Responses Against the Ancestral Strain (Ancestral Strain Hu-1) and the Beta Variant (B.1.351) Compared to Conventional mRNA and Adenovirus Vaccines

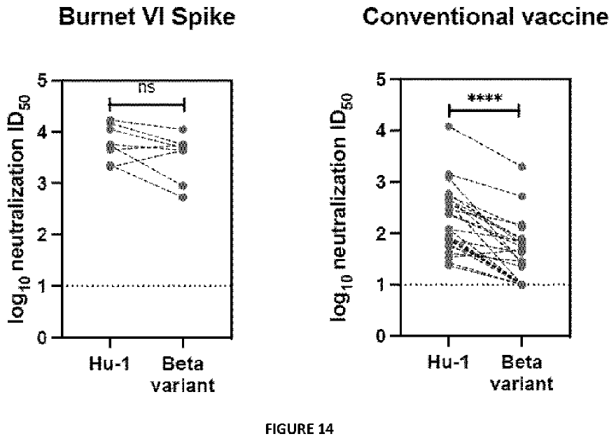

A pseudovirus neutralization ID 50 study was conducted to compare vaccine responses generated to S2P-FHA 1016/20VI spike protein versus vaccine responses generated in humans using conventional vaccines. Three doses of the S2P-FHA 1016/20VI protein vaccine in guinea pigs were compared to two doses of mRNA or adenovirus vaccines given to humans. The S genotypes used in the S-HIV pseudotype assays are shown below the graphs. Data shows neutralizing antibody responses against the matched WT virus and the highly resistant variant of concern (Beta Variant) from guinea pigs vaccinated with S2P-FHA protein trimers containing the 1016/20VI (Burnet VI spike on the left) compared to serum obtained 3-5 weeks after two doses of a conventional vaccine in humans (Conventional vaccine) on the right. A Wilcoxon matched pairs test was used to determine whether the differences in ID 50 s observed between groups was significant: ns, not significant, p≥0.05; ****, p<0.0001. As can be seen in FIG. 14, S2P-FHA 1016/20VI produces a greater neutralizing antibody response than the conventional mRNA and adenoviruses vaccines to both the ancestral strain and the beta VOC. The S2P-FHA 1016/20VI antigen produces a neutralizing antibody profile desirable for both primary vaccine regimens (the first vaccine regimen used to produce an immune response in a subject to SARS-CoV-2) and booster vaccine regimens. A booster vaccine may be used, for example, in a subject that has received a primary vaccine regimen that has produced one or more of: a response to the ancestral strain, a low response to the ancestral strain, a low immune response to a VOC, and a low immune response to the beta VOC.

### Example 10-Properties of Serum Antibodies Elicited by S2P-FHA, S2P.1016L-FHA and S2P.1016/20VI-FHA Trimer Immunogens in Experimental Animals

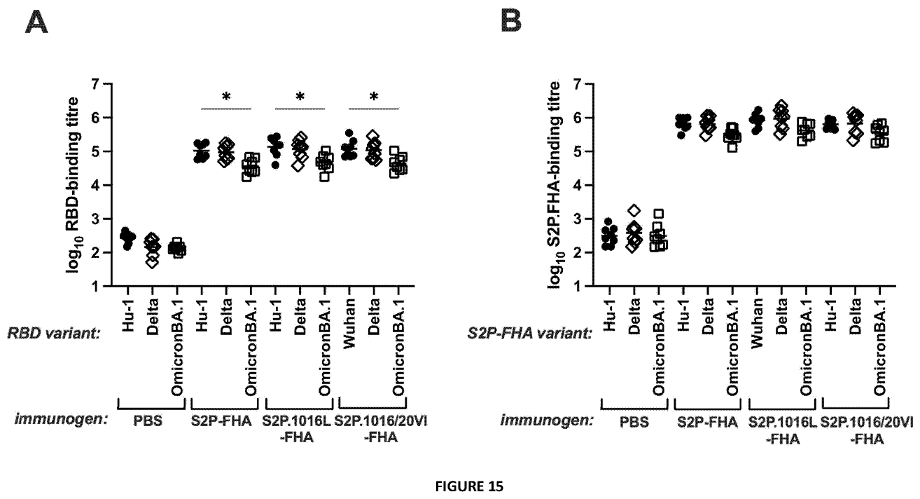

A capture ELISA format, employing plate-bound avidin to capture biotinylated Hu-1, Delta and Omicron BA.1 RBDs, was used to determine the RBD binding titres of vaccinal sera. The Delta (B.1.617.2) VOC emerged in October 2020 and became the dominant global variant. Two prominent mutations are present in the RBD, L452R and E484Q. Omicron (B.1.1.529), first identified in South Africa and Botswana (November 2021) has supplanted Delta variant in most countries. Omicron consists of at least three genetically distinct sub-lineages (BA.1, BA.2, and BA.3) that emerged in 2021. Initially, BA. 1 was the most common circulating version but the more infectious BA.2 variant has now become dominant. 30 mutations occur in Spike including 15 in the RBD and 8 in the NTD, which contain the major sites of neutralization. FIG. 15A show no significant differences between immunogen groups with geometric mean binding titers in the range 3.5×10⁵-1.36×10⁵. A small (˜3-fold) but significant reduction in binding titre was observed for the omicron BA.1 RBD for the 3 immunogen groups. Serum binding titres to Hu-1, Delta and Omicron BA.1 S2P-FHA spike protein trimers bound directly to ELISA plates were next determined. FIG. 15 B reveals no significant differences in binding titres to the 3 S2P-FHA trimer variants for the 3 immunogen groups. S2P-FHA binding titres were in the range 3.0×10⁵-1.1×10⁶. These results suggest that all immunogens generated similar titres of antibody able to bind the S2P-FHA spike trimer.

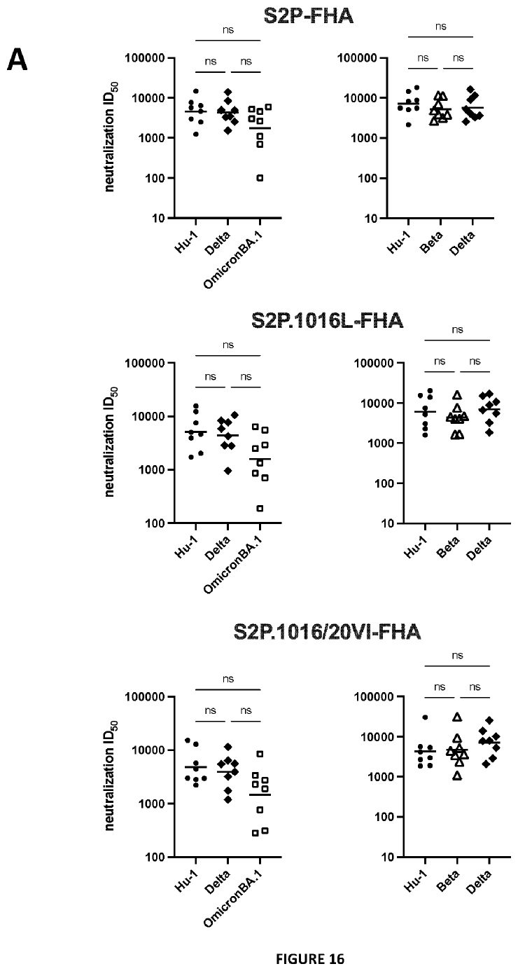

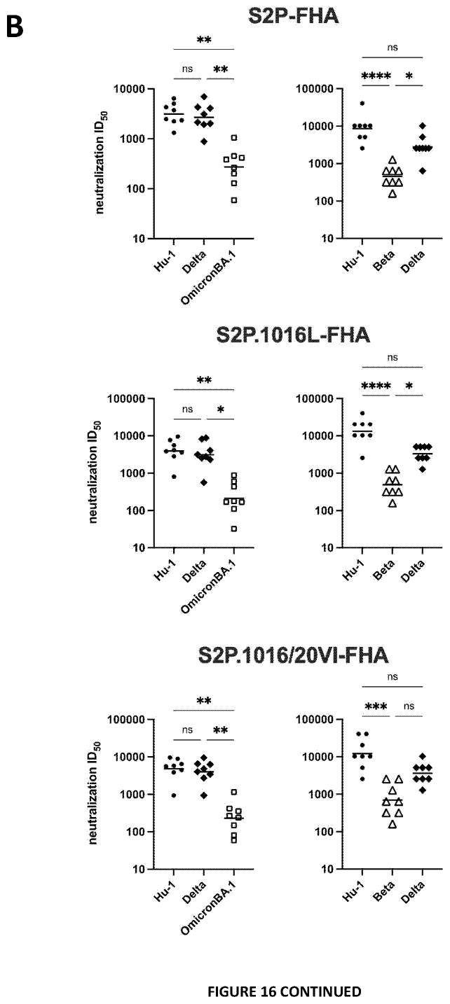

The neutralizing activity in vaccinal sera was next determined using S-pseudotyped HIV luciferase reporter viruses and 293-ACE2 target cells as described by Jackson et al. 2020. Pseudoviruses containing the S2P immunogen-matched Hu-1 Spike glycoprotein or that of Delta indicated potent neutralizing activity in S2P-FHA-, 1016L- and 1016/20VI-immune sera with mean ID₅₀s ranging from 3,900-5,100 (FIG. 16A). Serum neutralizing activities against pseudoviruses containing the Omicron BA.1 or Beta Spikes were not significantly different relative to Hu-1 and Delta pseudovirus neutralization, although the IC₅₀s against omicron BA.1 pseudoviruses trended downwards with <3-fold reductions in mean titre observed for the 3 groups.

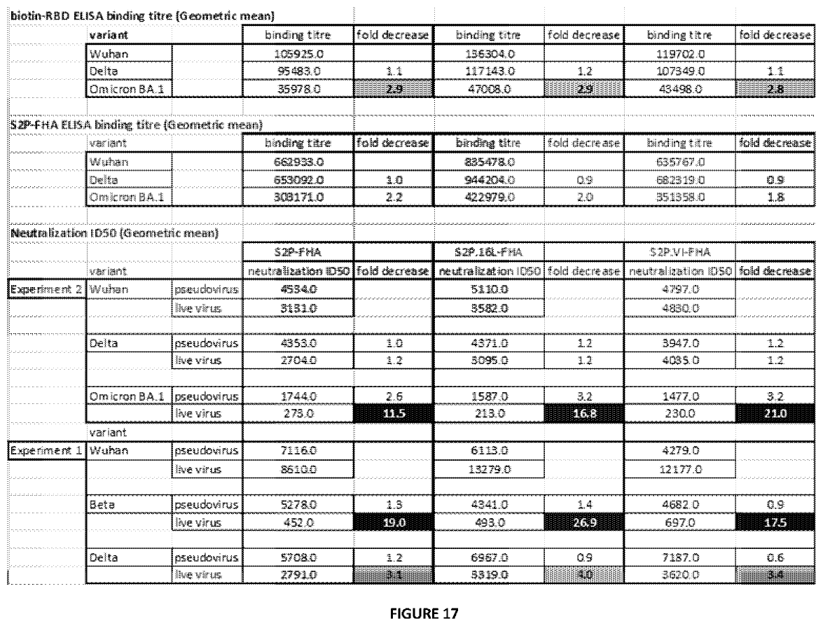

We next tested neutralizing activity against bona fide infectious viruses of Hu-1, Delta, Omicron BA.1 and Beta SARS-CoV-2 using HAT-24 cells in the R-20 microneutralization assay developed by Aggarwal et al. 2021. Whereas Hu-1 and Delta viruses were potently neutralized by sera from the 3 immunogen groups, Omicron BA.1 and Beta virus neutralization was slightly reduced by ˜1 log₁₀ (FIG. 16 B). FIG. 17 shows a summary of all geometric mean titres obtained in ELISA (RBD and Spike trimers), pseudotype virus neutralization and microneutralization assays. Overall, the data indicate that 3 doses of S2P-FHA, S2P.1016L-FHA and S2P.1016/20VI-FHA elicit high-titre antibodies against the RBD and S trimer that are able to potently neutralize HIV pseudoviruses containing the matched ancestral Hu-1 Spike as well as pseudoviruses carrying Spikes from the Delta, Omicron BA. 1 and Beta VOCs. High neutralization titres were also observed with bona fide infectious SARS-CoV-2 although moderate ˜1 log₁₀ reductions in ID₅₀ were observed with the Omicron BA.1 and Beta variants.

### Example 11—Assessment of Whether Mutations in the Alanine Cavity have a General Stabilizing Effect when Introduced to Spike Trimers Derived from Highly Divergent SARS-CoV-2 Sequences

Thirty mutations occur in the omicron BA.1 Spike with 8 in the N-terminal domain (NTD) and 15 in the RBD when compared to the ancestral sequence of Hu-1 (Jung et al. 2022). The NTD and RBD contain the major neutralization epitopes and consistent with the presence of multiple mutations in these 2 domains, sera obtained from mRNA doubly vaccinated individuals retain very little neutralizing activity against Omicron BA.1 viruses in vitro (Tada et al. 2022), corresponding to low vaccine efficacy against omicron infection (Andrews et al. 2022).

The mutations present in the Omicron BA.1 Spike (compared to Ancestral Hu-1) are:
NTD: A67V; deletion of H69-V70; T95I; G142D; delV143-Y145; deletion of 211; L212I; insertion of EPE after residue 214;
RBD: G339D; S371L; S373P; S375F; K417N; N440K; G446S; S477N; T478K; E484A; Q493R; G496S; Q498R; N501Y; Y505H;
C-terminal region of S1: T547K; D614G; H655Y; N679K; P681H;
S2: N764K; D796Y; N856K; Q954H; N969K; L981F

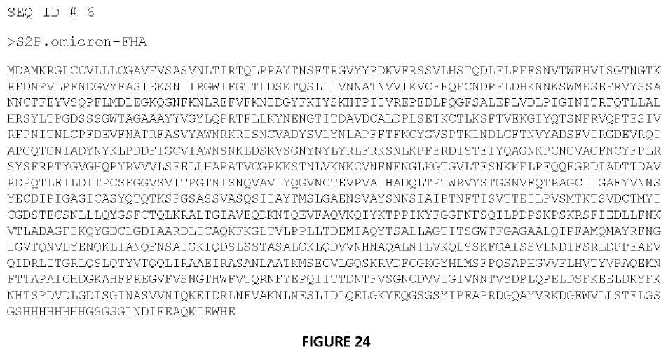

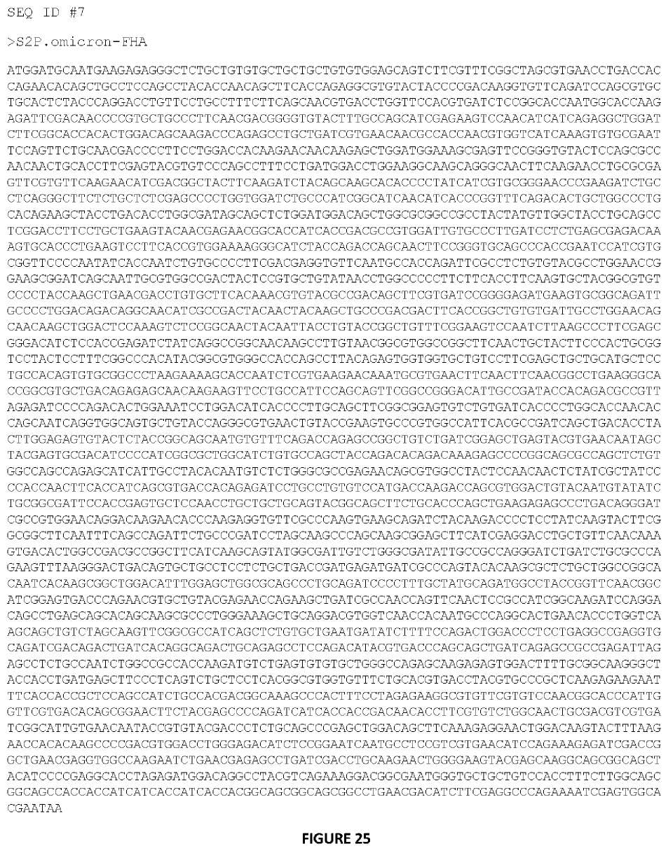

To determine whether mutations in the alanine cavity have a general stabilizing effect when introduced to Spike trimers derived from highly divergent VOC sequences, Omicron BA.1 versions of S2P-FHA and S2P.1016/20VI-FHA constructs were prepared (sequences in FIGS. 24, 25) and are referred to here as S2P.omicron-FHA and S2P.omicron.VI-FHA, respectively. The S2P.omicron-FHA sequences comprise the tissue plasminogen activator leader sequence upstream of S residues 16-1208, a furin cleavage site mutation, H681RRAR->P681GSAS, a di-Pro substitution at positions 986 and 987. Foldon, octa-His and avitag sequences were added to the C-terminus. The DNA sequences were cloned into a CMV promoter-driven expression vector and the proteins were expressed in Expi293F cells by transfection.

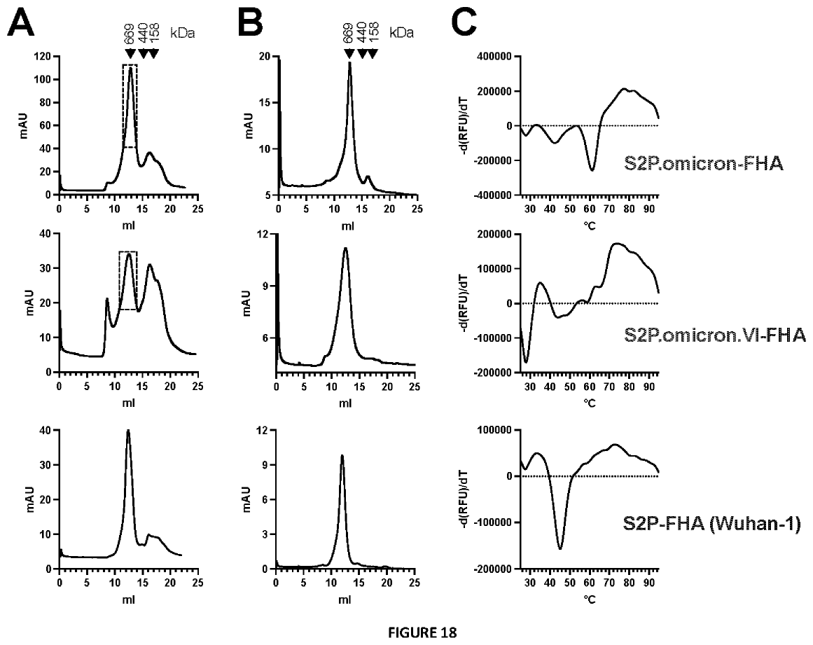

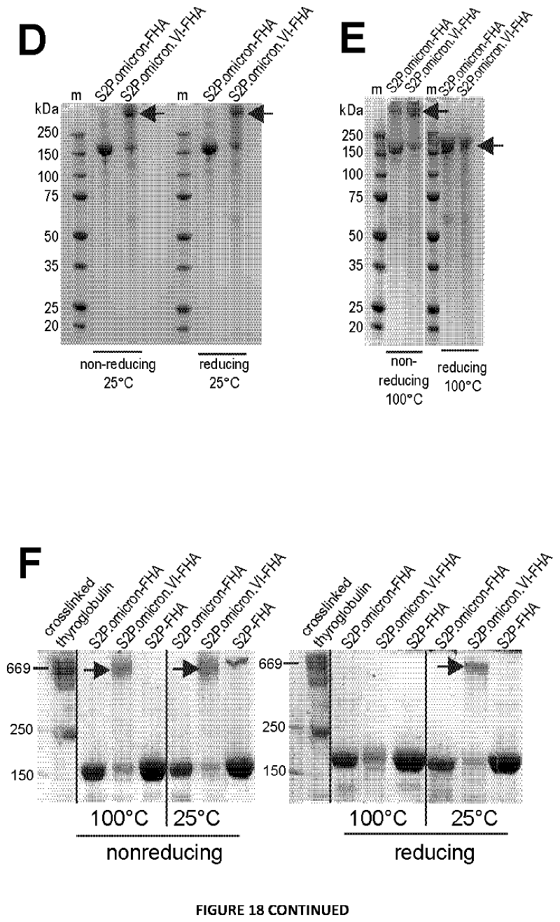

The proteins were extracted from the culture supernatants by divalent cation affinity chromatography and further purified by Superose 6 SEC. S2P.omicron-FHA eluted as a major peak close to the position of thyroglobulin (669 kDa) (FIG. 18A, top) and had an almost identical profile to that of S2P-FHA derived from the Hu-1 isolate (FIG. 18A, bottom). The trimer was collected as a homogenous protein as indicated by analytical SEC (FIG. 18 B). S2P.omicron.VI-FHA eluted as 4 species, however, a prominent presumed trimer peak was observed (indicated by a dashed box in FIG. 18A, middle. Fractions corresponding to trimer (dashed box in FIG. 18A) were pooled and concentrated and re-chromatographed in a Superose 6 column, revealing a largely homogeneous species corresponding to the trimer (FIG. 18 B; FIG. 18 C, top).

A thermofluor assay indicated that the S2P.omicron-FHA trimer possessed relatively high thermal stability with a melting temperature of 61° C. (FIG. 18 C, top). S2P-FHA derived from Hu-1, had a melting temperature of 43° C. (FIG. 18 C, bottom), consistent with FIG. 4C. Thermal unfolding was not observed for S2P.omicron.VI-FHA (FIG. 18 C, middle).

The purified S2P.omicron-FHA and S2P.omicron. VI-FHA trimers were further analysed by SDS-PAGE under non-reducing and reducing (1% betamercaptoethanol) conditions in the absence of sample boiling (FIG. 18 D). The S2P.omicron-FHA trimer was largely resolved to its expected monomer molecular weight of ˜ 160 kDa in both the presence and absence of reducing agent. By contrast, the major S2P.omicron.VI-FHA species (indicated by an arrow) was retained as a high molecular weight species migrating close to the top of the gel; a minor species was also observed at the monomer position. FIG. 18 E shows a repeat experiment in which the samples were boiled for 5′ prior to electrophoresis. Under non-reducing conditions, the results seen in FIG. 18 D were largely recapitulated with the high molecular weight major S2P.omicron.VI-FHA species again observed. However, boiling in the presence of reducing agent resolved this species to its monomer molecular weight. The data (FIG. 18 E) suggest that the S2P.omicron. VI-FHA trimer resists disruption by 0.8% w/v sodium dodecyl sulfate denaturant with and without 1% betamercaptoethanol at 25° C. The data further suggest that the S2P.omicron.VI-FHA trimer resists disruption by 0.8% w/v sodium dodecyl sulfate denaturant following boiling. The S2P.omicron.VI-FHA trimer is only resolved to a monomer after boiling in the presence of both 0.8% w/v sodium dodecyl sulfate denaturant and 1% betamercaptoethanol. The data suggest that the addition of the VI mutations into the alanine cavity of S2 confer hyperstability to the S2P.omicron. VI-FHA trimer enabling the trimer structure to withstand greater denaturing forces.

Thyroglobulin was covalently crosslinked with bis(sulfosuccinimidyl)suberate to obtain an SDS-PAGE marker with a theoretical mol. wt of 669 kDa, which is close to that of the S trimer. 18 Figure F shows crosslinked that thyroglobulin with co-migrated the SDS/betamercaptoethanol-resistant high molecular weight form of S2P.omicron.VI-FHA following treatment with 0.8% SDS at 25° C. or 100° C. for 3 min, or 0.8% SDS+1% betamercaptoethanol at 25° C. for 3 min prior to electrophoresis. Again, S2P.omicron.VI-FHA resolved as a monomer following boiling in 0.8% SDS+1% betamercaptoethanol. S2P.omicron-FHA and S2P-FHA proteins migrated as monomers following all treatments except for some residual trimeric S2P-FHA after treatment with 1% SDS at 25° C.

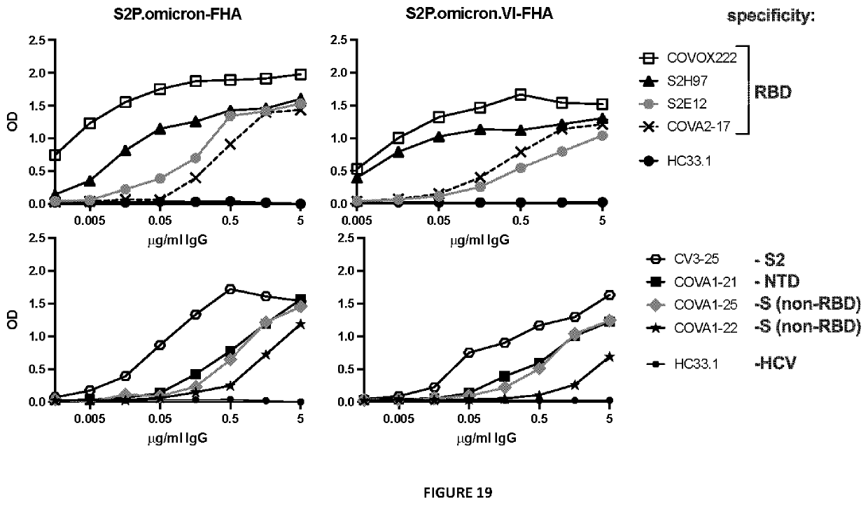

A capture ELISA employing plate-bound streptavidin to capture biotinylated S2P.omicron-FHA or biotinylated S2P.omicron.VI-FHA trimers (FIG. 19, left and right panels, respectively) was used to determine whether these proteins could be recognised by conformation dependent NAbs. The data show that the RBD-directed NAbs COVOX222, S2H97, and to a lesser extent, S2E12 and COVA2-17 bound to the 2 proteins with similar activity. Binding was also observed by CV3-25, a NAb to S2, COVA1-21 to the NTD and non-RBD directed NAbs, COVA1-25 and COVA1-22. HC33.1 is a hepatitis C virus NAb that serves as a negative control. The data indicate that S2P.omicron. VI-FHA trimers retain conformation dependent NAb epitopes also observed in S2P.omicron-FHA.

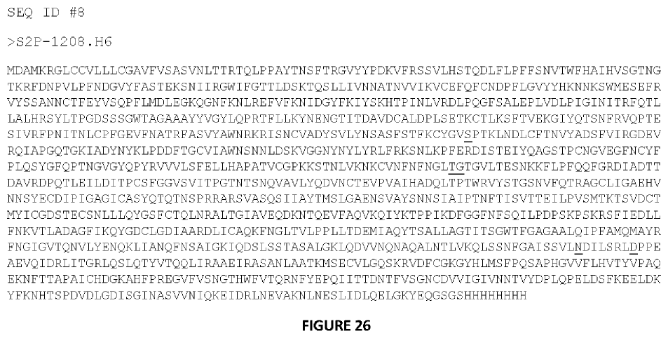

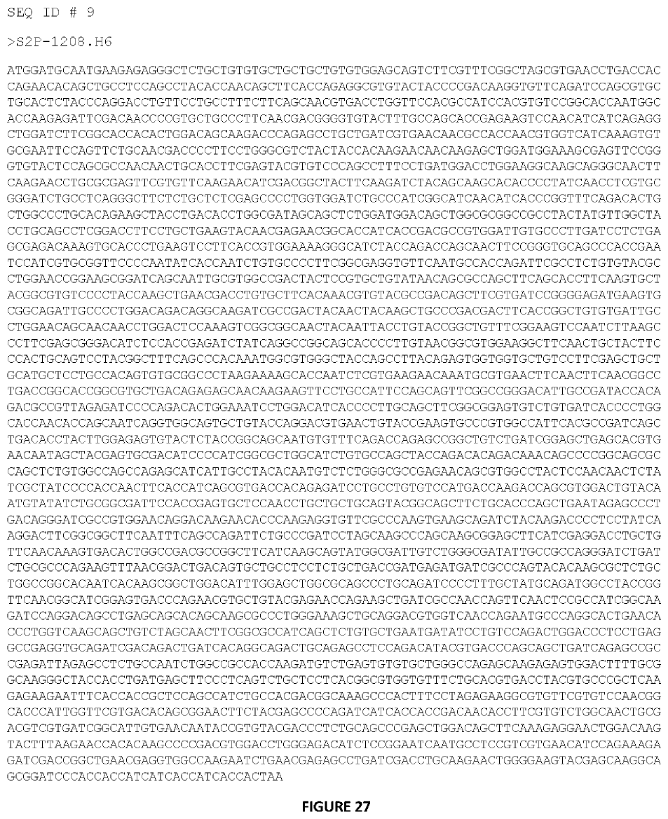

S2P-FHA engineered using the sequence of the ancestral Hu-1 SARS-CoV-2 sequence and variants of concern engineered here, in common with soluble S trimers described in the literature (e.g. Wrapp et al. 2020), contain a C-terminal trimerization module to stabilise the trimeric structure. This module is usually the trimeric foldon domain of bacteriophage T4 fibritin (FIG. 20A). NMR studies have shown that the native transmembrane domain is likely to be a triple stranded coiled-coil embedded in a lipid bilayer. To determine the oligomerization state of the Hu-1 S ectodomain in the absence of a trimerization tag, Hu-1 S2P was truncated at the last residue of the ectodomain, Q1208, to give S2P-1208.H6 (FIG. 20A; see FIGS. 26 and 27 for sequences). We also prepared the corresponding 1016/20VI mutant, S2P.VI-1208.H6. Both proteins have a hexa-His tag at the C-terminus to facilitate purification denoted by H6. The proteins were expressed in 293FS cells and then extracted from supernatants by divalent cation affinity chromatography. Superose 6 SEC revealed that Hu-1 S2P-1208.H6 eluted as a dimer in contrast to the trimeric structure of S2P-FHA (FIG. 20 B, top 2 panels). By contrast, trimers and dimers in roughly equal amounts were observed for Hu-1 S2P.VI-1208.H6 (FIG. 20 B, third panel). To enable the purification of S2P.VI-1208.H6 trimers, the protein was expressed in Expi293F cells and eluted from a HiTrap divalent cation-loaded column with an imidazole gradient. Superose 6 SEC indicated a prominent trimer peak for S2P.VI-1208-H6 (FIG. 20 B, fourth panel).

A thermofluor assay indicated that Hu-1 S2P-FHA trimers and S2P-1208.H6 dimers had a melting temperature of 43° C. whereas S2P.VI-1208.H6 comprised 2 species with melting temperatures of 43° C. and 58° C., respectively (FIG. 20 C). SDS-PAGE under nonreducing and reducing conditions confirmed that the protein preparations were of high purity and were denatured to monomers in 0.8% SDS and boiling with and without 1% betamercaptethanol indicating they are non disulfide linked trimers (FIG. 20 D). These data indicate that the 1016/20VI mutation confers sufficient stability to the S2P trimer obviating the need for a trimerization tag and confers thermal stability to the purified S2P oligomers.

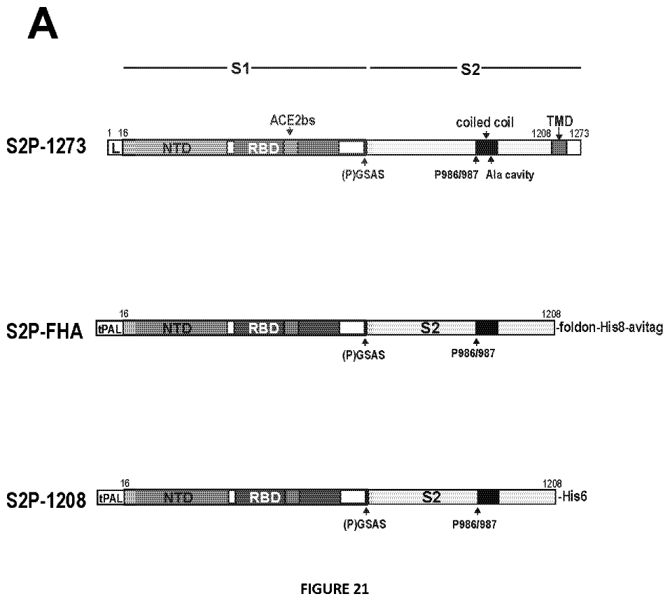

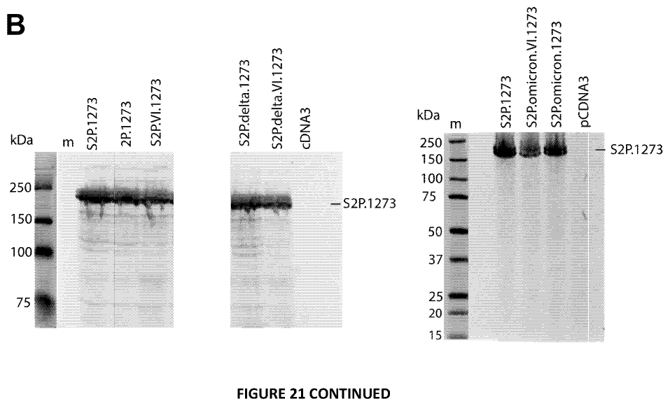

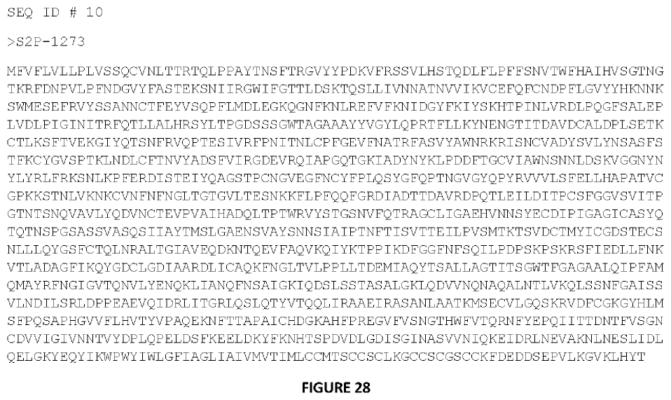

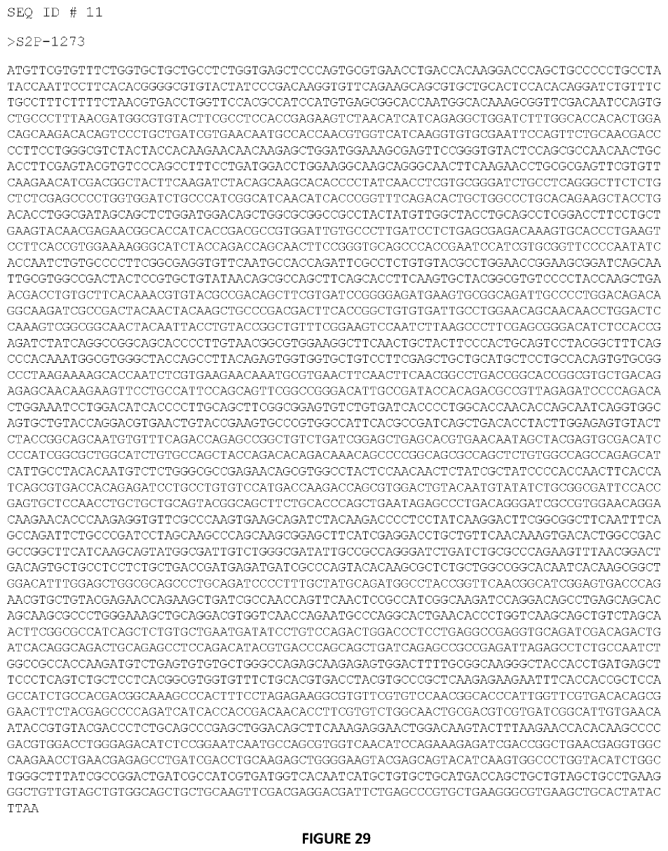

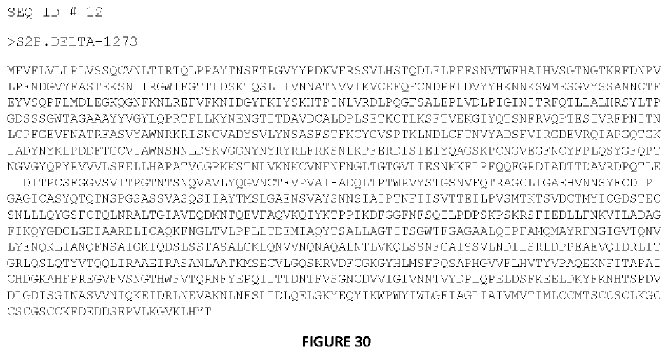

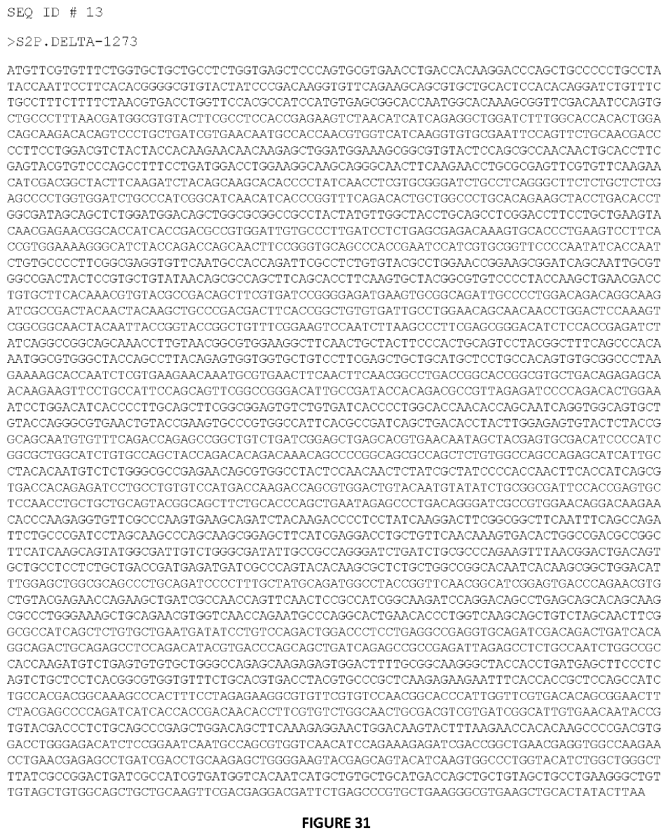

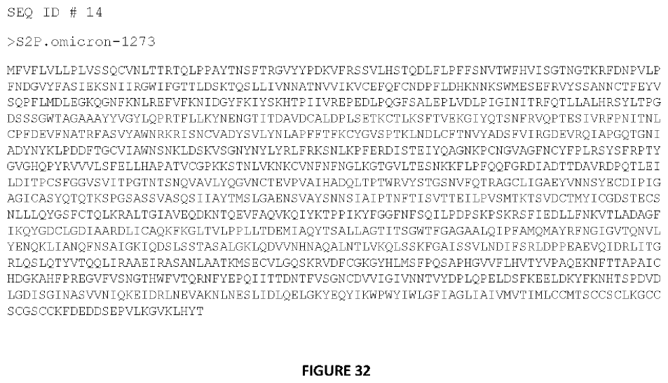

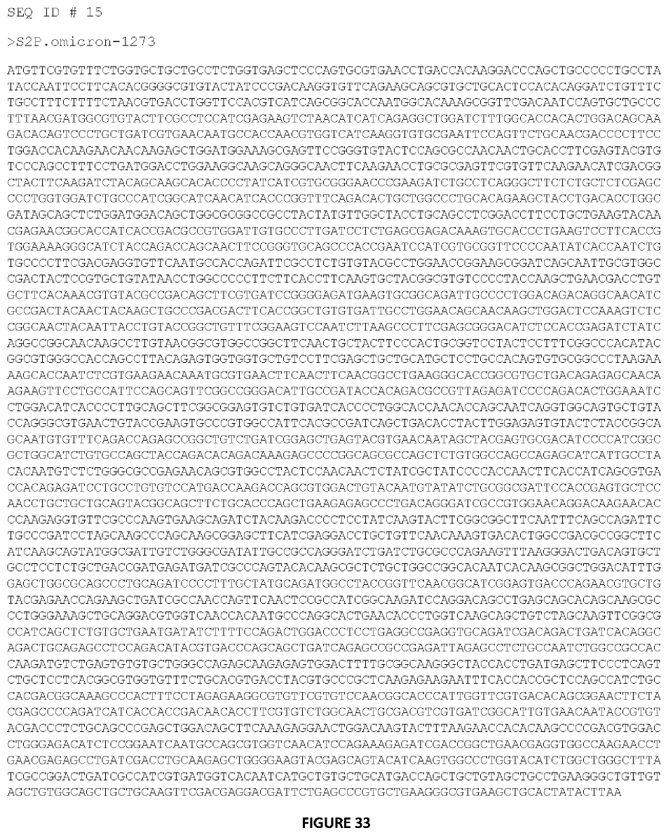

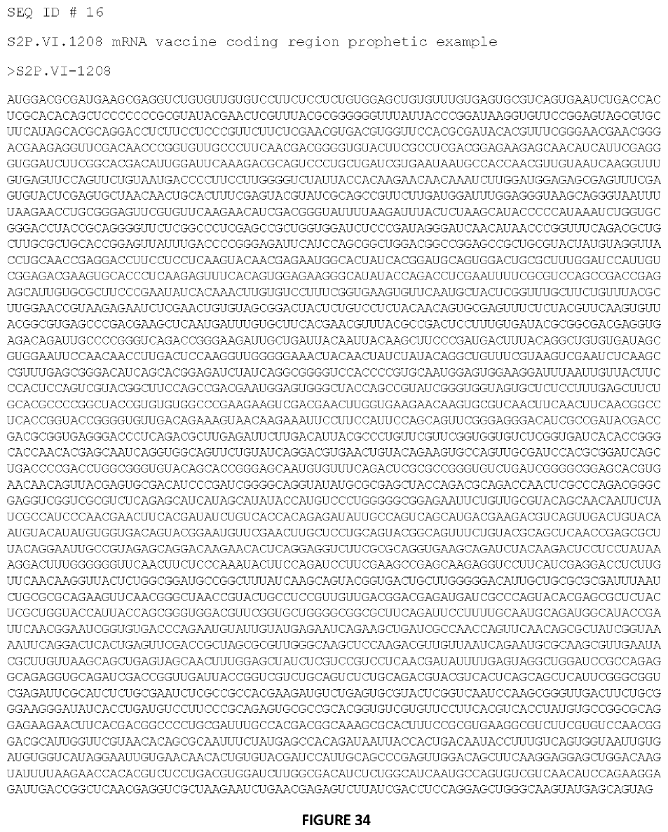

The effects of the 1016/20VI mutation in the context of full-length S2P containing the native transmembrane domain and cytoplasmic tail were examined. CMV driven expression vectors containing codon-optimised genes encoding residues 1-1273 of S glycoproteins derived from Hu-1, Delta and Omicron BA.1 were prepared. The R/H681RRAR->P681GSAS mutation at the furin site and the di-Pro “2P” substitution at positions 986 and 987 were also included. For a schematic of S2P-1273, see FIG. 21A; for protein and DNA sequences, see FIGS. 28-33.

To demonstrate expression of Hu-1, Delta and Omicron BA.1 S2P-1273 glycoproteins and 1016/20VI mutant versions thereof (S2P.VI-1273), the DNA vectors were transfected into 293T cells. The cells were lysed and the lysate subjected to SDS-PAGE and western blotting with rabbit anti-S1 polyclonal antibody. FIG. 21 B indicates a single ˜ 180 kDa protein band for each construct, consistent with that expected for the uncleaved S monomer. Transfection with an empty vector (pcDNA3) did not give rise to such a band.

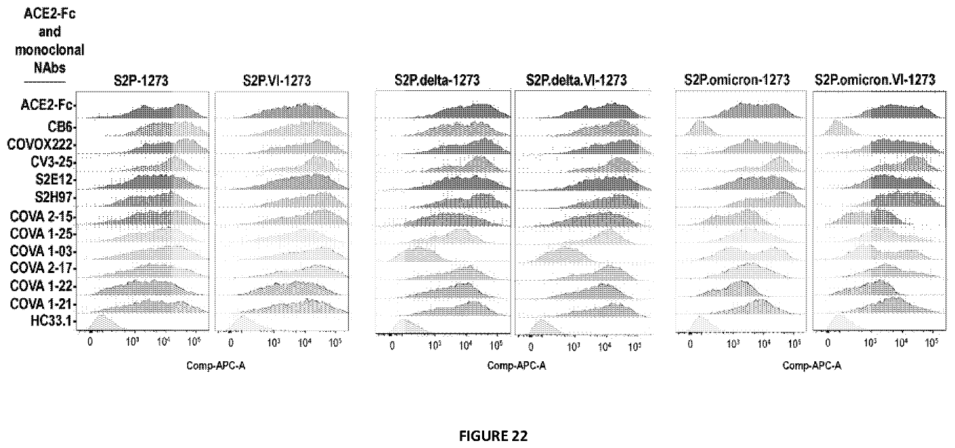

Flow cytometry was used to demonstrate that the Hu-1, Delta and Omicron BA.1 S2P.1273 and S2P.VI-1273 glycoproteins are expressed on the cell surface and are recognised by human monoclonal antibodies directed to key neutralization epitopes. 293T cells were transfected with the various S2P-1273 expression vectors and intact cells were stained with the various human monoclonal NAbs and AlexaFluor-conjugated anti-human immunoglobulin. The cells were counterstained with propidium iodide to enable the exclusion of dead cells from analyses. The histograms in FIG. 22 show that all expressed S2P-1273 glycoproteins bind to ACE2-Fc at similar levels and also to the majority of monoclonal NAbs tested. The isotype control HCV-specific antibody (HC33.1) exhibited no binding. The exceptions were CB6, which failed to bind to the Omicron spikes, whereas COVA 1-03 exhibited minimal binding to the Delta Spikes. These data indicate that the S2P-1273 glycoproteins are expressed on the cell surface and that they are competent to bind ACE2 receptor and retain diverse NAb epitopes.

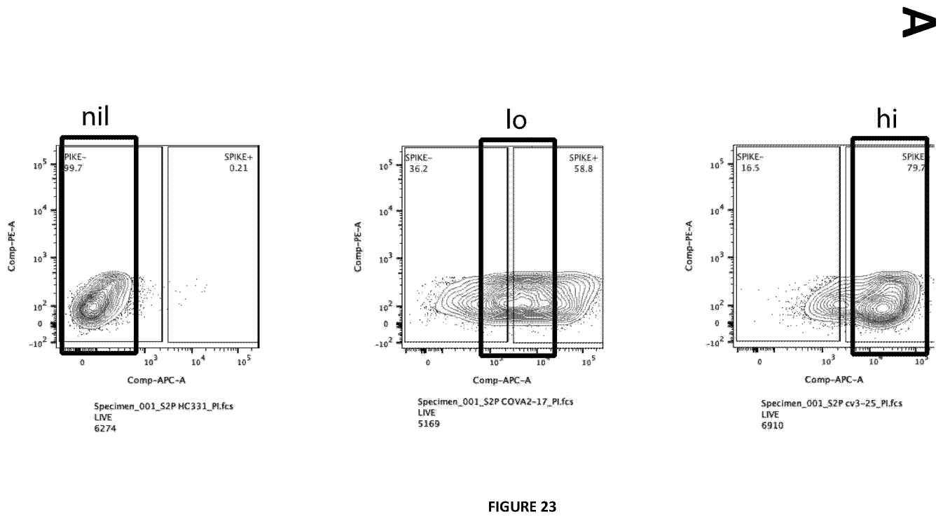

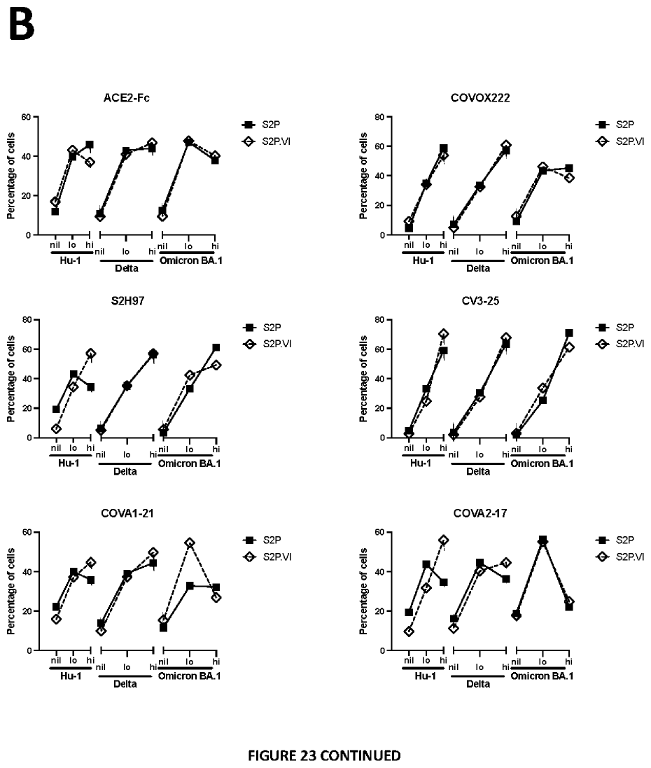

The data presented in FIG. 22 were further analysed using gates to distinguish non-fluorescing (nil), moderately (lo) fluorescent and highly (hi) fluorescent populations of cells (FIG. 23A). Introduction of the VI mutation in the context of S2P-1273 did not alter ACE2 receptor-binding ability nor exposure of a broadly neutralizing epitope in the RBD recognised by COVOX222 or an epitope within S2 recognised by CV3-25 (FIG. 23 B). However, VI increased the exposure of epitopes recognised by broadly neutralizing antibodies S2H97, COVA2-17 and COVA1-21 in S2P-1273 derived from Hu-1 ancestral, Delta, and Omicron BA.1 isolates. Thus the 1016/20VI mutation selectively enhances exposure of bNAb epitopes in an isolate-dependent manner.

### Example 12—The Effects of 1016/20VI Mutation on the Stability of Full-Length S2P-1273 and S2P-1208 Derived from Hu-1 and Omicron BA.1

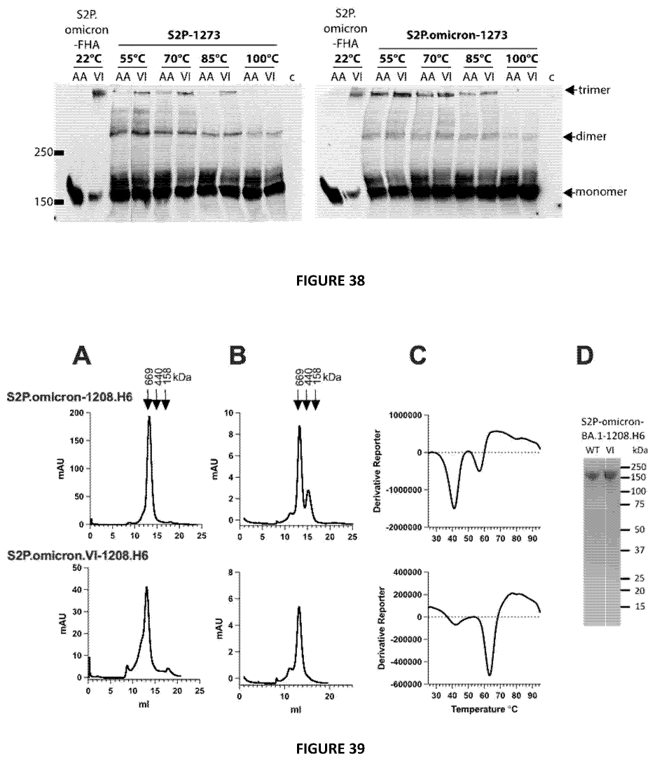

The effects of 1016/20VI mutation on the stability of full-length S2P-1273 (containing the native transmembrane domain and cytoplasmic tail) derived from Hu-1 and Omicron BA.1 (S2P-1273 and S2P.omicron-1273, respectively) and expressed in 293T cells were examined. Cell lysates were adjusted to contain a final concentration of 1.2% (w/v) SDS and 0.25% (v/v) betamercaptoethanol and treated at the indicated temperatures prior to SDS-PAGE and western blotting with rabbit anti-S1 polyclonal antibody. To mark the positions of monomer and trimer, respectively, AA and VI versions of purified S2P.omicron-FHA trimers treated with 0.67% SDS for 5 min at room temperature prior to electrophoresis (as for FIG. 18 F) were included. FIG. 38 reveals prominent S2P.omicron-FHA monomer and presumed S2P.omicron. VI-FHA trimer bands in lanes 2 and 3, respectively. In the case of S2P-1273 (FIG. 38, left panel), presumed trimer is observed following treatment at 55°, 70° and 85° C. when the 1016/20VI mutation is present, whereas this band is absent for S2P-1273 containing Ala at positions 1016 and 1020. The trimer band is absent following treatment at 100° C., suggesting dissociation. S2P.omicron-1273 trimers appeared to resist the 55°, 70° and 85° C. treatments irrespective of whether 1016/20VI was present, although slightly more trimer was observed with 1016/20VI (FIG. 38, right panel). These data are consistent with the higher overall thermostability observed for S2P.omicron-FHA relative to S2P-FHA derived from Hu-1. The data suggest that the VI mutation confers stability to S2P-1273 trimers as was observed with soluble S2P-FHA trimers. The data also indicate that S2P trimers derived from omicron BA.1 exhibit overall greater stability than Hu-1 S trimers.

Next, the effects of VI on the trimerization and stability of S2P-1208.H6 derived from the omicron BA.1 sequence, which lacks the foldon trimerization domain. S2P.omicron truncated at the last residue of the ectodomain, Q1208, was appended with GSGS-H6 at the C-terminus to give S2P.omicron-1208.H6 was examined. The corresponding 1016/20VI mutant, S2P.omicron.VI-1208.H6 was also prepared. The proteins were expressed in Expi293F cells and then extracted from supernatants by divalent cation affinity chromatography. Superose 6 SEC revealed that both proteins predominantly eluted as trimers (FIG. 39A). The purified trimers were reanalysed by Superose 6 SEC following a freeze (−80° C.)-thaw cycle revealing that ˜ 15% of the S2P.omicron-1208.H6 protein had dissociated to dimer. By contrast, the trimeric structure of S2P.omicron. VI-1208.H6 was retained (FIG. 39 B). A thermofluor assay indicated that the majority of purified S2P.omicron-1208.H6 had a melting temperature of 41° C. whereas the melting temperature of S2P.omicron.VI-1208.H6 trimers was much higher at 64° C. (FIG. 39 C). SDS-PAGE/Coomassie blue staining indicated that the purity of S2P.omicron-1208.H6 proteins approached 100% (FIG. 39 D). These data indicated that the VI mutation in the context of the Omicron BA.1 ectodomain enables highly stable soluble trimers to be obtained thereby obviating the requirement for an exogenous trimerization domain to maintain trimeric structure.

### Example 13. Effects of VI on the Exposure of Epitopes Recognised by Broadly Neutralising Human mAbs and ACE2-Fc in S2P-FHA Trimers Derived from Hu-1 and Omicron BA.1

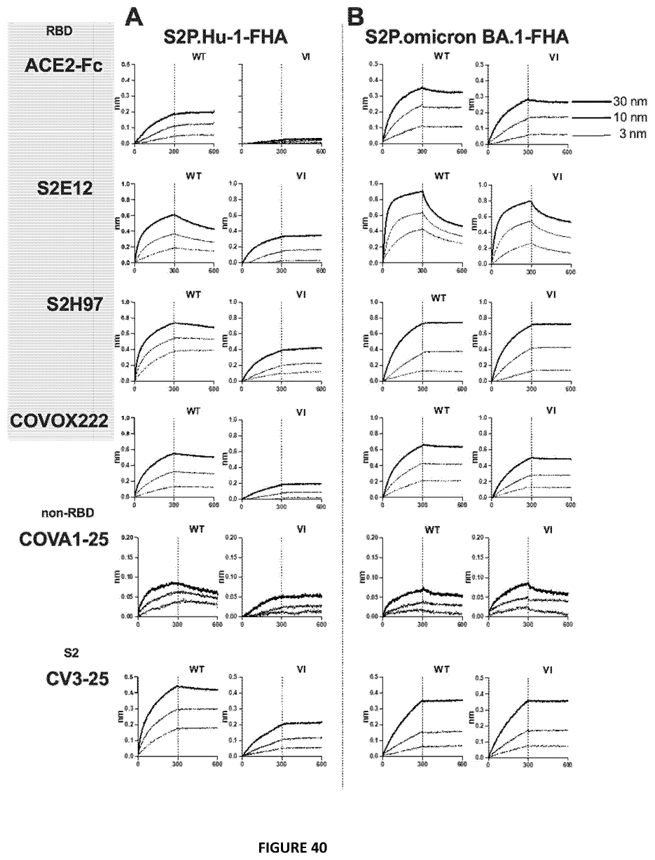

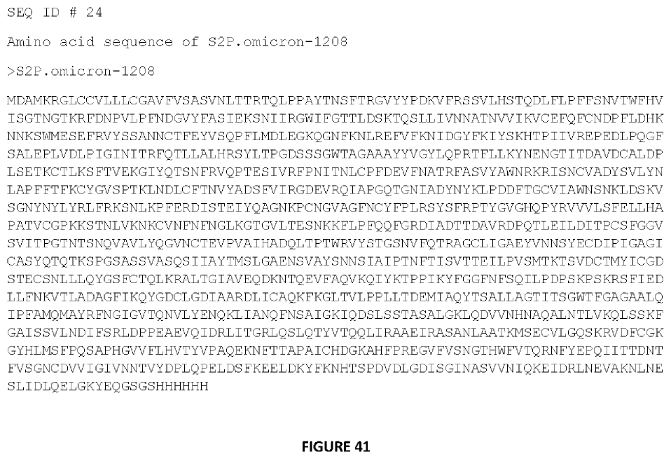

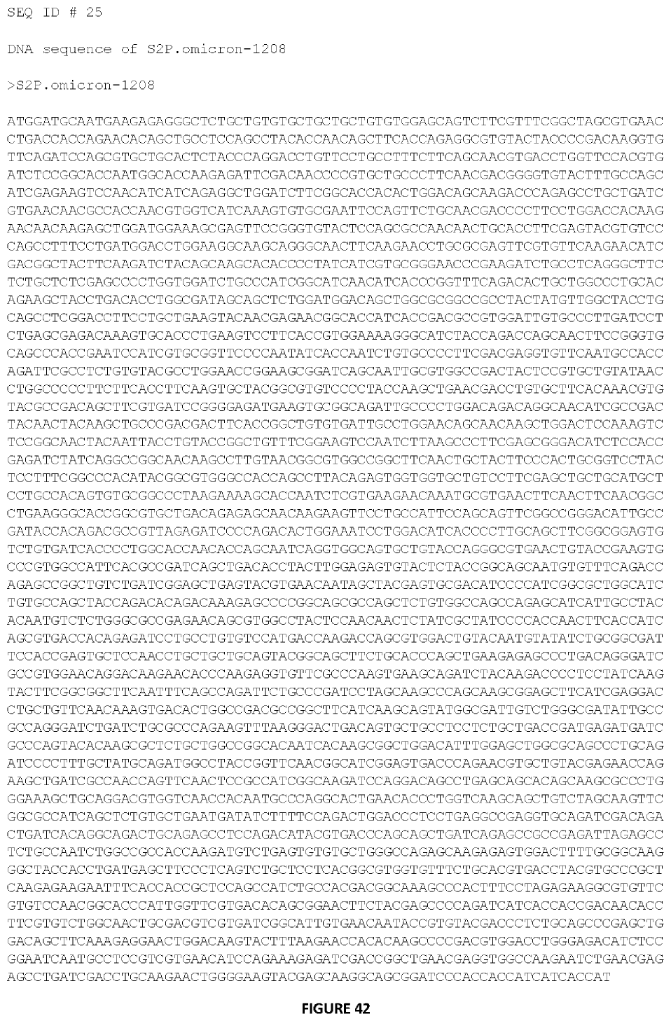

Next, biolayer interferometry was used to compare the effects of VI on the exposure of epitopes recognised by broadly neutralising human mAbs and ACE2-Fc in S2P-FHA trimers derived from Hu-1 and Omicron BA.1. ACE2-Fc and the mAbs were attached to anti-human IgG Fc capture biosensors while the S2P trimers were in the analyte phase. A comparison of sensograms indicated decreased overall binding by Hu-1-derived S2P.VI-FHA to RBD-directed ligands (ACE2-Fc, S2E12, S2H97 and COVOX222) relative to S2P-FHA (FIG. 40A). By contrast this VI-dependent decrease in binding to RBD ligands was not evident for S2P.omicron-FHA (FIG. 40 B). Hu-1 and Omicron BA.1-derived S2P-FHA proteins with and without VI bound to the same degree to COVA1-25, which recognises an epitope outside the RBD. CV3-25, which binds to an epitope in the S2 stem region, exhibited decreased binding to S2P.VI-FHA relative to the other proteins indicating that VI partially occludes this epitope in the context of Hu-1 but not Omicron BA.1. These data suggest that 1016/20VI promotes the RBD-down conformation in Hu-1 S2P-FHA trimers but not Omicron BA.1-FHA trimers. Thus, VI stabilises S2P.omicron trimers which also maintain exposure of key broad neutralization epitopes within the RBD. As the omicron strain mutations are likely to be widely represented in emerging strains, the results support the use of the modified antigen disclosed herein to provide effective immunity to emerging strains.

### Example 14—Results Summary

The coiled-coil at the center of the SARS-CoV-2 and SARS-CoV prefusion trimers is formed by 3 bow-shaped helices that expand away from each other from a point of contact mediated by the inward-facing Ile1013 together with Leu1012. The remainder of the 3-4 repeat is largely comprised of polar residues that mediate few inter-helical contacts. The inventors considered this polar topology might contribute to the relatively low thermostability of the prefusion trimer (observed to 43.6° C. in the studies described herein). SARS-CoVs are respiratory pathogens and the lower body temperatures at these sites of replication ranging from 20.5-35.5° C. may allow the Spike trimers to maintain their structure and function in a sustained manner. However, exposure to higher body temperatures as would be the case with intramuscular vaccination could lead to destabilization of the trimeric spike and loss of conformation over time, which would compromise immunogenicity.

Efforts to overcome the inherent instability of class I fusion proteins in their pre-fusion state through structure-based design has been adopted extensively for HIV-1, Ebola, respiratory syncytial virus, Lassa virus, human metapneumonia virus and coronaviruses. Previous studies discovered that introduction of two-proline residues in the hinge-loop between the central coiled-coil-forming helix CH1 and heptad repeat 1 stabilised the S proteins of SARS-CoV and middle eastern respiratory syndrome virus (MERS) stabilized the pre-fusion trimer conformation without loss of receptor binding or antigenicity. In the case of, SARS-CoV-2 Spike, prefusion stabilization was achieved by introduction of the S2-P mutation and ablation of the Arg682ArgAlaArg furin cleavage site (Wrapp et al., 2020), the latter not being present in SARS-CoV nor MERS Spike. Current human SARS-CoV-2 vaccines either contain the parental S sequence (Oxford/AstraZeneca (Watanabe et al., 2021)) or the S-2P/furin mutant (BioNTech/Pfizer, Johnson and Johnson (Bos et al 2020; Vogel et al., 2021)) or S2P with an intact furin site (Moderna (Jackson et al., 2020). The experiments described herein demonstrate that the S2P S trimer can be further stabilized and display enhanced stability and antigenic function by the creation of an artificial hydrophobic core in the center of the S2 coiled-coil of CH1 helices. In one embodiment, an artificial hydrophobic core can be generated by replacing Ala1016 and Ala1020 with bulkier hydrophobic residues to fill the cavity associated with these residues. Mutagenesis of Ala1016 (replacing the residue with a more hydrophobic residue) the shifted melting temperature from 43.6° C. to 58° C. species with A1016L giving the highest proportion of the 58° C. species. By contrast, the 43.6° C. species remained the predominant form with substitutions of Ala1020. Double substitutions were all associated with the more stable form, except for 1016/20VV. Interestingly, increased thermostability was sometimes associated with decreased soluble S2P-FHA expression, the lowest yields being obtained with 1016/20II, 1016/20LL, 1016/20VF, 1016/20IF and 1016/20FF. The 1016L and 1016/20VI mutants produced high yields suggesting that the bulk and/or geometry of the sidechain chosen to fill the cavity can impact the folding of the S2P trimer, with some sidechains having more favourable effects on folding. In the case of the Omicron BA.1 VOC, the 1016/20VI mutation stabilised S2P-FHA trimers against severe denaturing conditions such as boiling in the presence of 0.8% SDS or exposure to 0.8% SDS plus reducing agent betamercaptoethanol at ambient temperature, consistent with a hyperstable S2P trimer. The data indicate that creation of an artificial hydrophobic core in the centre of the CH coiled-coil of S2 improves stability and expression. These mutations can be combined with known stabilizing mutations e.g. S-2P and furin site mutation to enhance the biophysical properties of coronavirus spike protein vaccines.

The stabilizing 1016L and 1016/20VI had a measurable effect on the antigenicity of Hu-1 SARS-CoV-2 spike trimers. Biolayer interferometry revealed that the ACE2 extracellular domain and monoclonal antibodies that block RBD-ACE2 interactions had reduced binding to 1016L and 1016/20VI S2P trimers suggesting that the RBD-down conformation had been induced. Conversely, COVA1-25 that is directed to an epitope in S that is external to the RBD displayed improved binding to 1016L and 1016/20VI S2P trimers suggesting enhanced exposure of this domain in S. These effects of the stabilizing mutations on RBD orientation are likely to occur via allostery whereby changes to the geometry or conformation of the coiled-coil are transmitted through the trimer structure to the remote RBDs. These changes to RBD orientation in the context of S2P trimers did not translate to differences in RBD, S1 nor S2P trimer-directed antibody titres induced by the 3 immunogens examined here. Furthermore, the S2P-FHA, 1016L and 1016/20VI trimers generated comparably high NAb titres against pseudotypes packaged with the strain-matched Hu-1 Spike after 2 vaccinations, which was further enhanced with three vaccinations. However, consistent with an altered antigenic landscape, 1016/20VI-immune sera retained neutralization potency against beta/B.1.351 S variant pseudotypes, whereas this activity was significantly diminished for S2P-FHA and 1016L-elicited sera. These data with 1016/20L contrast those obtained with human vaccinal sera from humans vaccinated with BioNTech/Pfizer, Moderna or Oxford/Astrazeneca vaccines showing between 7.6 to 42-fold decreases in neutralization titre for beta/B.1.351 (Dejnirattisai et al., 2021; Garcia-Beltran et al., 2021). The beta variant has been shown to exhibit the greatest resistance to NAbs elicited by natural infection or vaccination when compared to other VOC including alpha/B.1.1.7, gamma/P.1 and delta/B.1.617 (Dejnirattisai et al., 2021; Garcia-Beltran et al., 202118, 21; Hoffmann et al., 2021). The differences in beta/B.1.351 variant neutralizing ability observed with S2P-FHA, 1016L and 1016/20VI were not reflected in differences in binding to the Hu-1 RBD nor RBD-NKY containing the beta/B. 1.351 mutations K417N, E484K, N501Y. These data illustrate that the S2P vaccines elicit RBD-directed antibody specificities that do not target epitopes containing the key residues mutated in the beta/B.1.351 VOC. Furthermore, serum neutralization potency was not affected by selected individual mutations present in VOCs again suggesting that these antibodies target residues other than those in the RBD that are mutated in VOCs. This observation is consistent with the data obtained in serum-NAb cross-competition assays indicating high titres of serum antibodies able to block binding by ACE2 and NAbs (CB6, B38, COVA2-15) directed to the RBD, COVA1-22, directed to the NTD as well as COVA1-25 directed to an S1 epitope that is outside the RBD and NTD. Thus, all three immunogens elicited a polyclonal response to the S trimer which may explain why the sera retain at least some potency against the beta/B.1.351 variant. That the mechanism whereby 1016/20VI-immune sera retain neutralization potency against the beta/B.1.351 variant was not explained by the serum-NAb competition assays may be due to the reliance of this assay on large IgG molecules present in sera to sterically block the binding of human IgG molecules to the S2P trimer. Overall, the three antigens appear to have similar abilities to induce antibodies directed to epitopes that overlap with the ACE2 binding site and the NTD but the competition ELISA is insufficiently sensitive to measure subtle differences in antibody binding modes. Thus, the tendency for the RBD-down conformation in 1016/20VI did not adversely affect its ability to induce NAbs and seemed to correlate with superior NAb activity against a neutralization-resistant VOC.

Current SARS-CoV-2 vaccines deliver ‘ancestral’ Spike sequences derived from the original Hu-1 isolate and program the cells of the vaccine recipient to produce the Spike protein which evokes an antibody response. The successful vaccination of the global human population has been complicated by high transmission rates in unvaccinated populations, enabling the evolution and spread of VOCs. Break-through infections by VOCs in vaccinated individuals has raised the major concern that SARS-CoV-2 may overcome vaccine-induced immunity as it continues to replicate, evolve and spread. Five major VOCs have spread through the human population: alpha (UK origin), beta (South African origin), gamma (Brazilian origin) and delta (Indian origin). The enhanced transmissibility properties of the alpha and delta variants enabled them to supplant ancestral Hu-1-derived virus lineages and to spread throughout the globe.—Omicron (B.1.1.529) is even more transmissible than Delta and is now the dominant VOC in most countries. Initially, BA.1 was the most common circulating version but the more infectious BA.2 subvariant has now become dominant. Other subvariants such as BA.4, BA.5 and BA.2.12.1 continue to emerge. Recent publications have indicated that the effectiveness of AstraZeneca and Pfizer-BioNTech vaccines is slightly decreased for alpha and delta variants relative to Hu-1 lineages, indicating that immunity elicited by these vaccines retains effectiveness against these VOCs (Emary et al, 2021; Lopez et al, 2021). In contrast to alpha and delta, the beta and gamma variants are more localised in distribution but exhibit properties that render vaccine induced immunity less effective. For example, a trial in South Africa revealed that the efficacy of the AstraZeneca vaccine against the beta VOC was only 10.4% (Madhi et al., 2021). This finding is particularly alarming as the theoretical emergence of a variant with combined high transmissibility and vaccine evading properties would render the vaccinated population susceptible to COVID-19. Extreme variation in the key neutralization sites of the Omicron Spike has led to greatly diminished the efficacy of 2-dose vaccination regimes employing on ancestral sequences, necessitating the use of boosters to recall protective immune responses (Andrews et al. 2022; Magen et al. 2022). The data presented here show that although the neutralization potency of vaccinal sera raised to the stabilised S2P-FHA proteins is only moderately diminished against the Beta and Omicron VOCs, the addition of stabilised Spikes derived from divergent VOCs such as Omicron to conventional vaccines based on ancestral sequences may broaden immunity for protection against emerging VOCs. Booster vaccines that broaden immunity against VOCs as they inevitably become endemic in the human population are therefore critical for long term control of COVID-19. The 1016/20VI Spike provides a strategy for developing a universal S booster vaccine that focuses the antibody response to highly conserved regions located outside the RBD, in addition to the RBD.

The tendency for the RBD-down conformation in S-based vaccines such as 1016/20VI could also be significant with respect to the balance of neutralizing and infection-enhancing antibodies being elicited. Complicating the development of a SARS-CoV vaccine is evidence suggesting that S-directed antibodies can enhance virus entry and promote acute lung injury via Fc effector mediated mechanisms. Antibody-dependent enhancement of infection can occur through receptor mimicry where NAbs bind to the RBD, priming the S trimer for fusion activation. Virus internalisation occurs through cell surface Fcgamma receptor interactions resulting in virus fusion and entry. Studies of monoclonal IgGs isolated from COVID-19 patients have revealed that antibodies to one group of RBD epitopes can mediate ADE via an Fcgamma receptor-dependent mechanism. Alternatively, antibodies against the NTD can induce the up RBD conformation, enhancing the ACE2 binding and infectivity. Another study found that whereas select RBD NAbs and non-neutralizing NTD antibodies demonstrated Fcgamma receptor-mediated enhancement of virus infection in vitro, both types of antibodies protected from SARS-CoV-2 replication in monkeys and mice. These data imply that the observation of ADE in vitro does not necessarily predict this process in vivo. Nevertheless, locking RBDs in the down conformation in a trimeric Spike vaccine such as 1016/20VI could favour the generation of neutralizing RBD-directed antibodies over ADE-antibody specificities.

While the unusual alanine cavity in the centre of SARS-CoV strains such as SARS-CoV-2 S2 can as shown herein be filled by the substitution of hydrophobic residues at amino acid positions 1016 and 1020, it is possible that additional or alternative a and d residues in the CH coiled-coil could be mutated to create additional stabilizing mutations through structure guided mutagenesis. Stabilizing the coiled-coil has multiple advantages over the existing di-proline and furin mutagenesis approaches by increasing thermal stability and locking down RBDs to prevent the potential generation of infection-enhancing antibody specificities. Furthermore, for SARS-COV-2 it is shown herein that spike trimers comprising alanine cavity mutations such as 1016/20VI elicit improved neutralization breadth, strength and longevity. Introduction of mutations such as 1016/20VI as described herein provides a strategy to improve the nature, durability and robustness of the NAb response against strains of SARS-CoV including, importantly, emerging VOCs to future proof vaccines for SARS-CoV-2. Indeed, the results show that introduction of the 1016/20VI into the omicron VOC has a stabilising effect to the S2P trimer obviating the need for a trimerization tag and confers thermal stability to the purified S2P oligomers. Thermal and denaturation stability is vital to meet global vaccine supply chain needs, while also providing benefits in vitro and in vivo. In one embodiment, the modified antigen obviates the need for a heterologous trimerization tag reducing the risk of off target reactivity.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2021902500 entitled “Vaccine Antigen” filed on 11 Aug. 2021, Australian Provisional Application No. 2021902530 entitled “Vaccine Antigen” filed on 13 Aug. 2021, and PCT Application No. PCT/AU2022/050429 entitled “Vaccine Antigen” filed on 6 May 2022 the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abu-Raddad et al. 2021. N Engl J Med doi: 10.1056/NEJMc2104974.
Aggarwal et al. 2021. doi: 10.21203/rs.3.rs-1210846/v1.
Alter et al. 2021. Nature doi: 10.1038/s41586-021-03681-2.
Andrews et al. 2022. doi: 10.1056/NEJMoa2119451
Andreano et al. 2020. bioRxiv doi: 10.1101/2020.12.28.424451.
Baden et al. 2021. N Engl J Med 384:403-416.
Bos et al. 2020. NPJ Vaccines 5:91.
Brouwer et al. 2020. Science 369:643-650.
Bullough et al. 1994. Nature 371:37-43.
Cai et al. 2020. Science 369:1586-1592.
Center et al. 2020. J Biol Chem 2020, 295, (21), 7179-7192.
Cerutti et al. 2021. Cell Host Microbe doi: 10.1016/j.chom.2021.03.005.
Chan et al. 1997. Cell 89:263-73.
Dejnirattisai et al. 2021. Cell doi: 10.1016/j.cell.2021.03.055.
Duan et al. 2020. Immunol 11:576622.
Emary et al. 2021. Lancet doi: 10.1016/S0140-6736 (21) 00628-0.
Finkelstein et al. 2021. Structural Analysis of Neutralizing Epitopes of the SARS-CoV-2 Spike to Guide Therapy and Vaccine Design Strategies. Viruses 13.
Fou et al. 2021. J Am Chem Soc 143:8543-6; doi: 10.1021/jacs. 1c02394.
Fu and Chou 2021. J Am Chem Soc 143:8543-8546.
Garcia-Beltran et al. 2021. Cell doi: 10.1016/j.cell.2021.03.013.
Guthe et al. 2004. J Mol Biol 337:905-15; doi: 10.1016/j.jmb.2004.02.020.
Hoffmann et al. 2021. Cell Rep 36:109415.
Hoffmann et al. 2020. Mol Cell 78:779-784 e5.
Huang et al. 2021. Curr Op in Coll & Inter Sci. https://doi.org/10.1016/j.cocis.2021.101468.
Jackson et al. 2020. N Engl J Med 383:1920-1931.
Jennewein et al. 2021. bioRxiv doi: 10.1101/2021.03.23.436684.
Julien et al. 2013. Science 342:1477-83.
Jung et al. 2022. doi: 10.1128/jvi.02077-21.
Ke et al. 2020. Nature 588:498-502.
Kustin et al. 2021. Nat Med doi: 10.1038/s41591-021-01413-7.
Liang et al. 2020 Nov. 6; 11:589833. https://doi.org/10.3389/fimmu.2020.589833.
Liu et al. 2020. Nature 584:450-456.
Liu et al. 2021. N Engl J Med doi: 10.1056/NEJMc2102017.
Logunov et al. 2021. Lancet 397:671-681.
Lopez et al. 2021. N Engl J Med doi: 10.1056/NEJMoa2108891.
Madhi et al. 2021. N Engl J Med doi: 10.1056/NEJMoa2102214.
Magen et al. 2022. N Engl J Med doi: 10.1056/NEJMoa2201688.
McCallum et al. 2021. Cell doi: 10.1016/j.cell.2021.03.028.
Naldini et al. 1996. Science 272 (5259): 263-7.
Niesen et al. 2007. Nat Protoc 2, (9), 2212-21.
Piccoli et al. 2020. Cell 183:1024-1042 e21.
Plante et al. 2021. Cell Host Microbe doi: 10.1016/j.chom.2021.02.020.
Polack et al. 2020. N Engl J Med 383:2603-2615.
Sadoff et al. 2021. N Engl J Med 384:2187-2201.
Sanyaolu et al. 2021. Ther Adv Infect Dis. 8:20499361211024372. doi: 10.1177/20499361211024372.
Schoenmaker et al. 2021. Int J Pharm doi: 10.1016/j.ijpharm.2021.120586
Shi et al. 2020. Nature 584:(7819):120-124.
Starr et al. 2021. Nature 597:97-102 doi: 10.1038/s41586-021-03807-6.
Supasa et al. 2021. Cell 184:2201-2211 e7.
Tada et al. 2021. bioRxiv doi: 10.1101/2021.02.05.430003.
Vasireddy et al. 2021. J Clin Med Res. 13 (6): 317-325. doi: 10.14740/jocmr4518
Vogel et al. 2021. Nature 592:283-289.
Voysey et al. 2021. Lancet 397:99-111.
Walls et al. 2020. Cell 183:1735.
Walls A C et al. 2017. Proc Natl Acad Sci 114:11157-11162.
Watanabe et al. 2021. ACS Cent Sci 7:594-602.
Weissenhorn et al. 1997. Nature 387:426-30.
Wilson 1981. Nature 289:366-73.
Wrapp et al. 2020. Science 367:1260-1263.
Wu et al. 2020. Science 368:1274-1278.

1. A coronavirus (CoV) vaccine antigen comprising a CoV S protein trimer wherein the S protein trimer is modified to comprise a structural modification which reduces the size of the alanine cavity in the coiled-coil region of the S protein trimer and wherein the S protein trimer elicits neutralising antibody responses.

2. The CoV vaccine antigen of claim 1, wherein the structural modification is within the coiled-coil.

3. The CoV vaccine antigen of claim 1 or claim 2, wherein the structural modification creates an artificial hydrophobic core in the alanine cavity.

4. The CoV vaccine antigen of any one of claims 1 to 3, wherein the structural modification is one or more of: amino acid substitution, disulphide bond, hydrogen bond, pi stacking (TT-TT stacking), salt bridge, van der Waals interactions, or proline stabilisation.

5. The CoV vaccine antigen of any one of claims 1 to 4, wherein one or the:
i) thermal stability of the S protein trimer is increased relative to an S protein trimer without the structural modification; and
ii) denaturant stability is increased relative to an S protein trimer without the structural modification.

6. The CoV vaccine antigen of any one of claims 1 to 5, wherein the ACE2 receptor binding domain (RBD) of the S protein trimer is in a down (non-ACE2-binding ready) orientation.

7. The CoV vaccine antigen of any one of claims 1 to 6, wherein at least one amino acid in the coiled-coil region of a S protein monomer of the S protein trimer is substituted with a more hydrophobic amino acid.

8. The CoV vaccine antigen of claim 7, wherein at least two amino acids in the coiled-coil region of a S protein monomer of the S protein trimer are substituted with a more hydrophobic amino acid.

9. The CoV vaccine antigen of claim 7 or claim 8, wherein the at least one amino acid or at least two amino acids are in position a and/or d of the heptad repeat motif of the coiled-coil region of the S protein monomers.

10. The CoV vaccine antigen of any one of claims 1 to 9, wherein the more hydrophobic amino acid comprises one or more of the following properties:
i) a hydrophobicity greater than alanine;
ii) is a hydrophobic amino acid that is larger than alanine;
iii) a hydrophobicity greater than 47 at a pH of 2;
iv) a hydrophobicity greater than 41 at a pH of 7; and
v) is selected from: isoleucine, leucine, methionine, valine, phenylalanine, tyrosine and tryptophan.

11. The CoV vaccine antigen of any one of claims 1 to 10, wherein the S protein monomers in the S protein trimer are SARS-CoV-2 S protein monomers.

12. The CoV vaccine antigen of any one of claims 1 to 11, wherein the S protein monomers in the S protein trimer further comprise one or more of:
i) the VOC/VOI mutations: S13I, L18F, T19R, T20N, P26S, A67V, delH69-V70, D80A, T95I, D138Y, G142D, delY144, W152C, E154K, E156del, F157del, R158G, R190S, D215G, del242-245, D253G, R246I, K417N/T, N439K, L452R/Q, Y453F, S477N, T478K, E484K/Q, N501Y, F565L, A570D, D614G, H655Y, Q677H, P681H/R, I692V, A701V, T7161, F888L, D950N, S982A, T1027I, Q1071H, D1118H, A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F;
ii) the amino acid sequence of any one of SEQ ID NO:1 to SEQ ID NO:3, SEQ ID: NO 6, and SEQ ID NO:08 or a sequence at least 90% identical thereto;
iii) the amino acid sequence of any one of SEQ ID NO:1 to SEQ ID NO:3, SEQ ID NO:6 and SEQ ID NO:8 comprising more or more of the mutations listed in iii);
iv) S protein residues 1-1208 of a SARS-CoV-2 Variant of Concern;
v) S protein residues 1-1208 of a SARS-CoV-2 Variant of Interest;
vi) S protein residues 1-1208 a SARS-CoV-2 Variant of High Consequence; and
vii) the amino acid sequence of SEQ ID NO:25 or a sequence at least 90% identical thereto;
viii) the amino acid sequence of SEQ ID NO:25 comprising more or more of the mutations listed in ii).

13. The CoV vaccine antigen of any one of claims 1 to 12, wherein the vaccine antigen lacks one or both of: a transmembrane domain and a trimerization sequence.

14. The CoV vaccine antigen of any one of claims 1 to 12, wherein the vaccine antigen comprises one of both of: a transmembrane domain and a trimerization sequence.

15. The CoV vaccine antigen of any one of claims 7 to 14, wherein the at least one amino acid in the coiled-coil region of S protein monomer is A1016.

16. The CoV vaccine antigen of claim 15, wherein A1016 is substituted with valine.

17. The CoV vaccine antigen of any one of claims 7 to 16, wherein the at least one amino acid in the coiled-coil region of S protein monomer is A1020.

18. The CoV vaccine antigen of claim 17, wherein A1020 is substituted with isoleucine.

19. The CoV vaccine antigen of any one of claims 8 to 18, wherein the at least one amino acid in the coiled-coil region of S protein monomer are A1016 substituted with valine and A1020 is substituted isoleucine.

20. The CoV vaccine antigen of any one of claims 1 to 19, wherein the S protein trimer elicits one or more of the neutralising antibody responses:
a) neutralising antibodies directed at the RBD;
b) neutralising antibodies directed at the N-terminal domain (NTD); and
c) neutralising antibodies at an undefined epitope in S1 outside the RBD and NTD.

21. The CoV vaccine antigen of any one of claims 1 to 20, wherein the antigen produces broadly neutralising antibody responses against epitopes outside the RBD (e.g., amino acids 16 to 329 and 522 to 1208 of SEQ ID NO: 1 or amino acids 16 to 329 of SEQ ID NO:1).

22. The CoV vaccine antigen of any one of claims 1 to 21, wherein the antigen is a soluble antigen.

23. The CoV vaccine antigen of any one of claims 1 to 22, wherein the antigen is stabilised in a pre-fusion conformation.

24. The CoV vaccine antigen of any one of claims 1 to 23, wherein the antigen comprises one or more further modified regions to enhance stability and/or immunogenicity.

25. A vector or polynucleotide encoding the S protein monomer of the coronavirus (CoV) vaccine antigen of any one of claims 1 to 24.

26. The vector or polynucleotide of claim 25, comprising the polynucleotide sequences described herein or comprising codon optimised or analogs or modifications to naturally occurring bases within the polynucleotide sequences described herein.

27. The vector or polynucleotide of claim 25 or claim 26, wherein the vector or polynucleotide is a ribonucleic acid.

28. A lipid nanoparticle comprising the vector or polynucleotide of any one of claims 25 to 27.

29. A host cell comprising the vector or polynucleotide of any one of claim 25 or 27.

30. A method of producing the coronavirus (CoV) vaccine antigen of any one of claims 1 to 24 comprising culturing the host cell of claim 29 in culture medium.

31. The method of claim 30, further comprising isolating CoV vaccine antigen, S protein, vector or polynucleotide from the cell and/or cell culture medium.

32. A protein nanoparticle comprising the coronavirus (CoV) vaccine antigen of any one of claims 1 to 24.

33. A virus-like particle comprising the coronavirus (CoV) vaccine antigen of any one of claims 1 to 24.

34. A vaccine comprising the coronavirus (CoV) vaccine antigen of any one of claims 1 to 24, or the vector or polynucleotide encoding the S protein monomer of the coronavirus (CoV) vaccine antigen of any one of claims 25 to 27, or the protein nanoparticle of claim 32, or the virus-like particle of claim 33.

35. The vaccine of claim 34, wherein the vaccine is a booster vaccine.

36. The vaccine of claim 35, wherein the booster vaccine provides protection against one or more of a CoV: VOC, VOI and VHC.

37. The vaccine of any one of claims 34 to 36, wherein the vaccine provides protection against SARS-CoV-2.

38. The vaccine of any one of claims 34 to 37, wherein the vaccine is selected from a: a) mRNA based vaccine; b) DNA based vaccine; c) viral vector vaccine; d) inactivated vaccine; e) live attenuated vaccine; and f) protein subunit vaccine.

39. The vaccine of any one of claims 34 to 38, wherein the vaccine further comprises at least one further CoV vaccine antigen or a further vector or polynucleotide encoding and capable of expressing a CoV vaccine antigen.

40. The vaccine of any one of claims 34 to 39, wherein the vaccine comprises an adjuvant.

41. The vaccine of claim 40, wherein the adjuvant is selected from one or more of: an aluminium salt-based adjuvant, an emulsion adjuvants, and a TLR agonist.

42. The vaccine of any one of claims 34 to 41, wherein the vaccine comprises a lipid nanoparticle.

43. A method of inducing an immune response to a coronavirus (CoV) in a subject, the method comprising administering the vaccine of any one of claims 34 to 42.

44. A method of enhancing the immune response to coronavirus (CoV) in a subject, the method comprising administering the vaccine of any one of claims 34 to 42 to a subject.

45. A method of preventing or reducing the likelihood of a coronavirus (CoV) infection in a subject, the method comprising administering the vaccine of any one of claims 34 to 42 to a subject.

46. A method of preventing or, reducing the likelihood or severity of a symptom of a coronavirus (CoV) infection in a subject, the method comprising administering the vaccine of any one of claims 34 to 42 to a subject.

47. A method of reducing the severity and/or duration of a coronavirus (CoV) infection in a subject, the method comprising administering the vaccine of any one of claims 34 to 42 to a subject.

48. A method of preventing or reducing viral shedding in a human individual infected with a coronavirus (CoV), the method comprising administering the vaccine of any one of claims 34 to 42 to a subject.

49. The method of any one of claims 43 to 48, wherein the CoV is SARS-CoV-2.

50. The method of any one of claims 43 to 49, wherein the subject had a previous CoV infection.

51. The method of any one of claims 43 to 50, wherein the vaccine is administered after a subject has received a primary CoV vaccine regimen.

52. The method of any one of claims 43 to 50, wherein the vaccine is administered intramuscularly, intradermal, subcutaneously, intravenously, intra-arterially, intraperitoneal, intranasal, sublingual, tonsillar, orally, pulmonary, topical or other parenteral and mucosal routes.

53. The CoV vaccine antigen of any one of claims 1 to 24 for use in one or more of:
i) preventing or reducing the likelihood of a CoV infection in a subject;
ii) preventing or reducing the likelihood of severity of a CoV symptom in a subject,
iii) reducing the severity and/or duration of a CoV infection in a subject;
iv) preventing or reducing viral shedding in a subject; and
v) treating a CoV infection in a subject.

54. A kit, device, surface or strip comprising the coronavirus (CoV) vaccine antigen of any one of claims 1 to 24.

55. Use of the coronavirus (CoV) vaccine antigen of any one of claims 1 to 24 in the manufacture of a medicament for one or more of:
i) preventing or reducing the likelihood of a CoV infection in a subject;
ii) preventing or reducing the likelihood of severity of a CoV symptom in a subject,
iii) reducing the severity and/or duration of a CoV infection in a subject;
iv) preventing or reducing viral shedding in a subject; and
v) treating a CoV infection in a subject.

56. Use of the antigen or encoding sequence in the manufacture of a preparation for treating preventing or testing relative to coronavirus (CoV) infections in a population.

57. A method of producing a soluble S protein trimer, lacking a heterologous trimerization sequence, wherein the S protein trimer is modified to comprise a structural modification which reduces the size of the alanine cavity in the coiled-coil region of the S protein trimer and wherein the S protein trimer elicits neutralising antibody responses.


SEQUENCE LISTING (26 sequences)

SEQ ID NO: 1 - protein, 1272 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHY

SEQ ID NO: 2 - protein, 1215 aa, Severe acute respiratory syndrome coronavirus 2
MDAMKRGLCCVLLLCGAVFVSASVNLTTRQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 3 - protein, 1276 aa, Severe acute respiratory syndrome coronavirus 2
MDAMKRGLCCVLLLCGAVFVSASVNLTTRQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGSYIPEAPRDGQAYVRKDGEWVLLSTFLGSGSHHHHHHHHGSGSGLNDIFEAQKIEWHE

SEQ ID NO: 4 - DNA, 3648 bp, Severe acute respiratory syndrome coronavirus 2
  source 1..3648: mol_type = genomic DNA
atggatgcaatgaagagagggctctgctgtgtgctgctgctgtgtggagcagtcttcgtttcggctagcgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgaattccagttctgcaacgaccccttcctgggcgtctactaccacaagaacaacaagagctggatggaaagcgagttccgggtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtataacagcgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgtttcggaagtccaatcttaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgtccttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacttggagagtgtactctaccggcagcaatgtgtttcagaccagagccggctgtctgatcggagctgagcacgtgaacaatagctacgagtgcgacatccccatcggcgctggcatctgtgccagctaccagacacagacaaacagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaacggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaatgatatcctgtccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgcctccgtcgtgaacatccagaaagagatcgaccggctgaacgaggtggccaagaatctgaacgagagcctgatcgacctgcaagaactggggaagtacgagcaa

SEQ ID NO: 5 - DNA, 3834 bp, Severe acute respiratory syndrome coronavirus 2
  source 1..3834: mol_type = genomic DNA
atggatgcaatgaagagagggctctgctgtgtgctgctgctgtgtggagcagtcttcgtttcggctagcgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgaattccagttctgcaacgaccccttcctgggcgtctactaccacaagaacaacaagagctggatggaaagcgagttccgggtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtataacagcgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgtttcggaagtccaatcttaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgtccttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacttggagagtgtactctaccggcagcaatgtgtttcagaccagagccggctgtctgatcggagctgagcacgtgaacaatagctacgagtgcgacatccccatcggcgctggcatctgtgccagctaccagacacagacaaacagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaacggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaatgatatcctgtccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgcctccgtcgtgaacatccagaaagagatcgaccggctgaacgaggtggccaagaatctgaacgagagcctgatcgacctgcaagaactggggaagtacgagcaaggcagcggcagctacatccccgaggcacctagagatggacaggcctacgtcagaaaggacggcgaatgggtgctgctgtccacctttcttggcagcggcagccaccaccatcatcaccatcaccacggcagcggcagcggcctgaacgacatcttcgaggcccagaaaatcgagtggcacgaataa

SEQ ID NO: 6 - protein, 1274 aa, Severe acute respiratory syndrome coronavirus 2
MDAMKRGLCCVLLLCGAVFVSASVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSPGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGSYIPEAPRDGQAYVRKDGEWVLLSTFLGSGSHHHHHHHHGSGSGLNDIFEAQKIEWHE

SEQ ID NO: 7 - DNA, 3825 bp, Severe acute respiratory syndrome coronavirus 2
  source 1..3825: mol_type = genomic DNA
atggatgcaatgaagagagggctctgctgtgtgctgctgctgtgtggagcagtcttcgtttcggctagcgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgaattccagttctgcaacgaccccttcctggaccacaagaacaacaagagctggatggaaagcgagttccgggtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtataacctggcccccttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtctccggcaactacaattacctgtaccggctgtttcggaagtccaatcttaagcccttcgagcgggacatctccaccgagatctatcaggccggcaacaagccttgtaacggcgtggccggcttcaactgctacttcccactgcggtcctactcctttcggcccacatacggcgtgggccaccagccttacagagtggtggtgctgtccttcgagctgctgcatgctcctgccacagtgtgcggccctaagaaaagcaccaatctcgtgaagaacaaatgcgtgaacttcaacttcaacggcctgaagggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacttggagagtgtactctaccggcagcaatgtgtttcagaccagagccggctgtctgatcggagctgagtacgtgaacaatagctacgagtgcgacatccccatcggcgctggcatctgtgccagctaccagacacagacaaagagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaagggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcaggacgtggtcaaccacaatgcccaggcactgaacaccctggtcaagcagctgtctagcaagttcggcgccatcagctctgtgctgaatgatatcttttccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgcctccgtcgtgaacatccagaaagagatcgaccggctgaacgaggtggccaagaatctgaacgagagcctgatcgacctgcaagaactggggaagtacgagcaaggcagcggcagctacatccccgaggcacctagagatggacaggcctacgtcagaaaggacggcgaatgggtgctgctgtccacctttcttggcagcggcagccaccaccatcatcaccatcaccacggcagcggcagcggcctgaacgacatcttcgaggcccagaaaatcgagtggcacgaataa

SEQ ID NO: 8 - protein, 1228 aa, Severe acute respiratory syndrome coronavirus 2
MDAMKRGLCCVLLLCGAVFVSASVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGSHHHHHHHH

SEQ ID NO: 9 - DNA, 3687 bp, Severe acute respiratory syndrome coronavirus 2
  source 1..3687: mol_type = genomic DNA
atggatgcaatgaagagagggctctgctgtgtgctgctgctgtgtggagcagtcttcgtttcggctagcgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgaattccagttctgcaacgaccccttcctgggcgtctactaccacaagaacaacaagagctggatggaaagcgagttccgggtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtataacagcgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgtttcggaagtccaatcttaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgtccttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacttggagagtgtactctaccggcagcaatgtgtttcagaccagagccggctgtctgatcggagctgagcacgtgaacaatagctacgagtgcgacatccccatcggcgctggcatctgtgccagctaccagacacagacaaacagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaacggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaatgatatcctgtccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgcctccgtcgtgaacatccagaaagagatcgaccggctgaacgaggtggccaagaatctgaacgagagcctgatcgacctgcaagaactggggaagtacgagcaaggcagcggatcccaccaccatcatcaccatcaccactaa

SEQ ID NO: 10 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - DNA, 3822 bp, Severe acute respiratory syndrome coronavirus 2
  source 1..3822: mol_type = genomic DNA
atgttcgtgtttctggtgctgctgcctctggtgagctcccagtgcgtgaacctgaccacaaggacccagctgccccctgcctataccaattccttcacacggggcgtgtactatcccgacaaggtgttcagaagcagcgtgctgcactccacacaggatctgtttctgcctttcttttctaacgtgacctggttccacgccatccatgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgaattccagttctgcaacgaccccttcctgggcgtctactaccacaagaacaacaagagctggatggaaagcgagttccgggtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtataacagcgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgtttcggaagtccaatcttaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgtccttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacttggagagtgtactctaccggcagcaatgtgtttcagaccagagccggctgtctgatcggagctgagcacgtgaacaatagctacgagtgcgacatccccatcggcgctggcatctgtgccagctaccagacacagacaaacagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaacggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcaggacgtggtcaaccagaatgcccaggcactgaacaccctggtcaagcagctgtctagcaacttcggcgccatcagctctgtgctgaatgatatcctgtccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgccagcgtggtcaacatccagaaagagatcgaccggctgaacgaggtggccaagaacctgaacgagagcctgatcgacctgcaagagctggggaagtacgagcagtacatcaagtggccctggtacatctggctgggctttatcgccggactgatcgccatcgtgatggtcacaatcatgctgtgctgcatgaccagctgctgtagctgcctgaagggctgttgtagctgtggcagctgctgcaagttcgacgaggacgattctgagcccgtgctgaagggcgtgaagctgcactatacttaa

SEQ ID NO: 12 - protein, 1271 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 3816 bp, Severe acute respiratory syndrome coronavirus 2
  source 1..3816: mol_type = genomic DNA
atgttcgtgtttctggtgctgctgcctctggtgagctcccagtgcgtgaacctgaccacaaggacccagctgccccctgcctataccaattccttcacacggggcgtgtactatcccgacaaggtgttcagaagcagcgtgctgcactccacacaggatctgtttctgcctttcttttctaacgtgacctggttccacgccatccatgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgaattccagttctgcaacgaccccttcctggacgtctactaccacaagaacaacaagagctggatggaaagcggcgtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtataacagcgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgtttcggaagtccaatcttaagcccttcgagcgggacatctccaccgagatctatcaggccggcagcaaaccttgtaacggcgtggaaggcttcaactgctacttcccactgcagtcctacggctttcagcccacaaatggcgtgggctaccagccttacagagtggtggtgctgtccttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacttggagagtgtactctaccggcagcaatgtgtttcagaccagagccggctgtctgatcggagctgagcacgtgaacaatagctacgagtgcgacatccccatcggcgctggcatctgtgccagctaccagacacagacaaacagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaacggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcagaacgtggtcaaccagaatgcccaggcactgaacaccctggtcaagcagctgtctagcaacttcggcgccatcagctctgtgctgaatgatatcctgtccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgccagcgtggtcaacatccagaaagagatcgaccggctgaacgaggtggccaagaacctgaacgagagcctgatcgacctgcaagagctggggaagtacgagcagtacatcaagtggccctggtacatctggctgggctttatcgccggactgatcgccatcgtgatggtcacaatcatgctgtgctgcatgaccagctgctgtagctgcctgaagggctgttgtagctgtggcagctgctgcaagttcgacgaggacgattctgagcccgtgctgaagggcgtgaagctgcactatacttaa

SEQ ID NO: 14 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSPGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3813 bp, Severe acute respiratory syndrome coronavirus 2
  source 1..3813: mol_type = genomic DNA
atgttcgtgtttctggtgctgctgcctctggtgagctcccagtgcgtgaacctgaccacaaggacccagctgccccctgcctataccaattccttcacacggggcgtgtactatcccgacaaggtgttcagaagcagcgtgctgcactccacacaggatctgtttctgcctttcttttctaacgtgacctggttccacgtcatcagcggcaccaatggcacaaagcggttcgacaatccagtgctgccctttaacgatggcgtgtacttcgcctccatcgagaagtctaacatcatcagaggctggatctttggcaccacactggacagcaagacacagtccctgctgatcgtgaacaatgccaccaacgtggtcatcaaggtgtgcgaattccagttctgcaacgaccccttcctggaccacaagaacaacaagagctggatggaaagcgagttccgggtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtataacctggcccccttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtctccggcaactacaattacctgtaccggctgtttcggaagtccaatcttaagcccttcgagcgggacatctccaccgagatctatcaggccggcaacaagccttgtaacggcgtggccggcttcaactgctacttcccactgcggtcctactcctttcggcccacatacggcgtgggccaccagccttacagagtggtggtgctgtccttcgagctgctgcatgctcctgccacagtgtgcggccctaagaaaagcaccaatctcgtgaagaacaaatgcgtgaacttcaacttcaacggcctgaagggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacttggagagtgtactctaccggcagcaatgtgtttcagaccagagccggctgtctgatcggagctgagtacgtgaacaatagctacgagtgcgacatccccatcggcgctggcatctgtgccagctaccagacacagacaaagagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaagggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcaggacgtggtcaaccacaatgcccaggcactgaacaccctggtcaagcagctgtctagcaagttcggcgccatcagctctgtgctgaatgatatcttttccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgccagcgtggtcaacatccagaaagagatcgaccggctgaacgaggtggccaagaacctgaacgagagcctgatcgacctgcaagagctggggaagtacgagcagtacatcaagtggccctggtacatctggctgggctttatcgccggactgatcgccatcgtgatggtcacaatcatgctgtgctgcatgaccagctgctgtagctgcctgaagggctgttgtagctgtggcagctgctgcaagttcgacgaggacgattctgagcccgtgctgaagggcgtgaagctgcactatacttaa

SEQ ID NO: 16 - RNA, 3651 nt, Severe acute respiratory syndrome coronavirus 2
  mRNA 1..3651: note = U indicates pseudouridine (p) or 1-methylpseudouridine (mlf)
  source 1..3651: mol_type = mRNA
atggacgcgatgaagcgaggtctgtgttgtgtccttctcctctgtggagctgtgtttgtgagtgcgtcagtgaatctgaccactcgcacacagctcccccccgcgtatacgaactcgtttacgcggggggtttattacccggataaggtgttccggagtagcgtgcttcatagcacgcaggacctcttcctcccgttcttctcgaacgtgacgtggttccacgcgatacacgtttcgggaacgaacgggacgaagaggttcgacaacccggtgttgcccttcaacgacggggtgtacttcgcctcgacggagaagagcaacatcattcgagggtggatcttcggcacgacattggattcaaagacgcagtccctgctgatcgtgaataatgccaccaacgttgtaatcaaggtttgtgagttccagttctgtaatgaccccttccttggggtctattaccacaagaacaacaaatcttggatggagagcgagtttcgagtgtactcgagtgctaacaactgcactttcgagtacgtatcgcagccgttcttgatggatttggagggtaagcagggtaattttaagaacctgcgggagttcgtgttcaagaacatcgacgggtattttaagatttactctaagcatacccccataaatctggtgcgggacctaccgcaggggttctcggccctcgagccgctggtggatctcccgatagggatcaacataacccggtttcagacgctgcttgcgctgcaccggagttatttgaccccgggagattcatccagcggctggacggccggagccgctgcgtactatgtaggttacctgcaaccgaggaccttcctcctcaagtacaacgagaatggcactatcacggatgcagtggactgcgctttggatccattgtcggagacgaagtgcaccctcaagagtttcacagtggagaagggcatataccagacctcgaattttcgcgtccagccgaccgagagcattgtgcgcttcccgaatatcacaaacttgtgtcctttcggtgaagtgttcaatgctactcggtttgcttctgtttacgcttggaaccgtaagagaatctcgaactgtgtagcggactactctgtcctctacaacagtgcgagtttctctacgttcaagtgttacggcgtgagcccgacgaagctcaatgatttgtgcttcacgaacgtttacgccgactcctttgtgatacgcggcgacgaggtgagacagattgccccgggtcagaccgggaagattgctgattacaattacaagcttcccgatgactttacaggctgtgtgatagcgtggaattccaacaaccttgactccaaggttgggggaaactacaactatctatacaggctgtttcgtaagtcgaatctcaagccgtttgagcgggacatcagcacggagatctatcaggcggggtccaccccgtgcaatggagtggaaggatttaattgttacttcccactccagtcgtacggcttccagccgacgaatggagtgggctaccagccgtatcgggtggtagtgctctcctttgagcttctgcacgccccggctaccgtgtgtggcccgaagaagtcgacgaacttggtgaagaacaagtgcgtcaacttcaacttcaacggcctcaccggtaccggggtgttgacagaaagtaacaagaaattccttccattccagcagttcgggagggacatcgccgatacgaccgacgcggtgagggaccctcagacgcttgagattcttgacattacgccctgttcgttcggtggtgtctcggtgatcacaccgggcaccaacacgagcaatcaggtggcagttctgtatcaggacgtgaactgtacagaagtgccagttgcgatccacgcggatcagctgaccccgacctggcgggtgtacagcaccgggagcaatgtgtttcagactcgcgccgggtgtctgatcggggcggagcacgtgaacaacagttacgagtgcgacatcccgatcggggcaggtatatgcgcgagctaccagacgcagaccaactcgcccagacgggcgaggtcggtcgcgtctcagagcatcatagcatataccatgtccctgggggcggagaattctgttgcgtacagcaacaattctatcgccatcccaacgaacttcacgatatctgtcaccacagagatattgccagtcagcatgacgaagacgtcagttgactgtacaatgtacatatgtggtgacagtacggaatgttcgaacttgctcctgcagtacggcagtttctgtacgcagctcaaccgagcgcttacaggaattgccgtagagcaggacaagaacactcaggaggtcttcgcgcaggtgaagcagatctacaagactcctcctataaaggactttggggggttcaacttctcccaaatacttccagatccttcgaagccgagcaagaggtccttcatcgaggacctcttgttcaacaaggttactctggcggatgccggctttatcaagcagtacggtgactgcttgggggacattgctgcgcgcgatttaatctgcgcgcagaagttcaacgggctaaccgtactgcctccgttgttgacggacgagatgatcgcccagtacacgagcgctctactcgctggtaccattaccagcgggtggacgttcggtgctggggcggcgcttcagattccttttgcaatgcagatggcataccgattcaacggaatcggtgtgacccagaatgtattgtatgagaatcagaagctgatcgccaaccagttcaacagcgctatcggtaaaattcaggactcactgagttcgaccgctagcgcgttgggcaagctccaagacgttgttaatcagaatgcgcaagcgttgaatacgcttgttaagcagctgagtagcaactttggagctatctcgtccgtcctcaacgatattttgagtaggctggatccgccagaggcagaggtgcagatcgaccggttgattaccggtcgtctgcagtctctgcagacgtacgtcactcagcagctcattcgggcggtcgagattcgcatctctgcgaatctcgccgccacgaagatgtctgagtgcgtactcggtcaatccaagcgggttgacttctgcgggaagggatatcacctgatgtccttcccgcagagtgcgccgcacggtgtcgtgttccttcacgtcacctatgtgccggcgcaggagaagaacttcacgacggcccctgcgatttgccacgacggcaaagcgcactttccgcgtgaaggcgtcttcgtgtccaacgggacgcattggttcgtaacacagcgcaatttctatgagccacagataattaccactgacaatacctttgtcagtggtaattgtgatgtggtcataggaattgtgaacaacactgtgtacgatccattgcagcccgagttggacagcttcaaggaggagctggacaagtattttaagaaccacacgtctcctgacgtggatcttggcgacatctctggcatcaatgccagtgtcgtcaacatccagaaggagattgaccggctcaacgaggtcgctaagaatctgaacgagagtcttatcgacctccaggagctgggcaagtatgagcagtag

SEQ ID NO: 17 - RNA, 3822 nt, Severe acute respiratory syndrome coronavirus 2
  mRNA 1..3822: note = U indicates pseudouridine (p) or 1-methylpseudouridine (mlf)
  source 1..3822: mol_type = mRNA
atgttcgtgttcctggtgctgctgccgttagtgagtagtcagtgtgtgaacctgactactcgcacgcagctgcctccagcgtacacgaacagctttaccaggggggtctactaccccgacaaggtgtttaggtcgagtgtgcttcactcgacccaggacctcttcctcccgttcttctcgaacgtgacgtggttccacgcgatacacgtttcgggaacgaacgggacgaagaggttcgacaacccggtattacccttcaacgatggggtctactttgcctcgacagagaagagtaacatcattcgtggctggatattcgggacaacattggactcgaagacgcagtccttgttgatcgtgaataacgccacgaatgttgttattaaggtctgcgagttccagttctgcaacgaccccttcttgggggtgtattaccacaagaacaacaagagctggatggaatccgagtttcgggtttattccagcgctaacaactgtacttttgagtatgtcagtcaaccgttcttgatggacctcgaaggtaagcaaggcaactttaagaaccttcgagagttcgtctttaagaacattgacggatatttcaagatatacagcaagcacacaccaatcaatttggtgcgtgacttgccccagggcttctctgccctggagccgttggtggaccttccaatcggcataaacatcactcggttccagaccctcctggctctccacaggtcttacctgacccctggagacagcagcagtgggtggactgcaggtgccgccgcgtactacgtggggtacctgcagccacgcacttttctgctgaagtataacgagaatggcacgatcacggacgctgtggattgtgcccttgatccactgtcagagactaagtgcactcttaagtcttttacagtggagaagggcatataccagacgtccaactttcgtgtccagcctactgagagtattgtgcgattcccgaatattacgaacctgtgtccgttcggcgaggtttttaatgcgacgcggttcgcaagcgtgtacgcttggaaccgcaagcgcattagtaactgcgtcgcggactacagcgttctgtataattcggcgtcgttcagtactttcaagtgctacggggtgtctcccacgaagctgaacgatctgtgtttcactaatgtgtacgcagactcgttcgttattcgtggcgacgaggtccggcagatcgctccgggccagacgggcaagattgcggattacaactataagctcccggacgacttcaccgggtgcgttatagcttggaattcgaacaatcttgactcgaaggtaggaggtaattacaattacctctaccgtctctttcgaaagtccaacttgaagcccttcgagcgggacatttcgacagagatataccaggcagggtccactccctgcaatggtgtagagggatttaactgttatttccctctacagagctacggcttccagccgacaaacggggtagggtaccagccctaccgcgtggtggtgctctcctttgagctcctccacgcgcccgcgactgtctgtgggcccaagaagtcgacgaacttggtgaagaacaagtgcgtcaacttcaacttcaatgggctcacaggcacgggggtcctcaccgagtcgaacaagaagtttcttccattccagcagtttggtcgcgacatcgcggacaccaccgacgcggtgcgcgacccccagacgctggaaatcttggatataactccttgttcgttcggtggggtgtcggtgatcacgcctgggacgaacacgagtaaccaggtggcagttctgtaccaggacgtgaactgtacagaagtgcccgtggctattcatgcggaccagctgacaccgacatggcgggtatatagcactgggtcgaatgtgtttcagacacgtgctgggtgcctgattggcgccgagcacgtgaacaattcgtacgagtgcgatatacccatcggggctgggatctgcgcgagctatcagacccagacgaactcgcctgggtctgctagctcggtagcttcccagtccattatcgcctacactatgtctttgggcgcagagaatagtgtggcgtatagcaataactccatcgcgataccgacgaacttcacgatcagtgtcacgacggagatattgcctgtcagcatgaccaagacgtcggtcgactgcacgatgtacatctgcggcgattcgaccgagtgcagcaacttgctgttgcagtacgggtcgttttgcacgcagttgaatcgtgcattgaccggcatcgcggtcgagcaggacaagaacacgcaggaggtgttcgcgcaggtcaagcagatctataagacccctcctatcaaggactttgggggctttaacttcagccagatcctgccggacccctccaagccgagcaagaggtcctttattgaggacctcttgttcaacaaagtcacgttggctgatgcggggtttatcaagcaatacggcgattgtctgggtgacatcgccgctagggacttgatatgcgcccagaagttcaacggacttacggtgttaccgcccctgctgaccgacgagatgatagcccaatatacatctgcgttgttagcagggacgataacatcgggttggacctttggtgcgggcgctgcattgcagatcccttttgcaatgcagatggcctaccggttcaacggaattggggtgactcagaacgttctctatgagaaccagaagctgatcgccaaccagttcaactcggcgatagggaagatccaggattccctatcgtcgaccgcgtctgcgcttggtaagctgcaggacgtggtcaaccagaatgctcaggcgttgaatacattggtgaaacagctctcgagtaatttcggagctatttcatcagtgttgaacgacatcttgagcaggctggaccctccggaggcggaggtccagatcgaccgtttgataactgggaggctgcagagcctccagacgtacgttacccagcagctcattagagctgtcgagatccggatttccgcaaatctagccgctacgaagatgtccgagtgcgttctcggacagtcgaagcgggtagatttttgcggaaaggggtaccatctgatgagcttcccgcagtccgccccgcatggagtggtcttcctgcatgtcacgtatgtgccggcgcaggagaagaacttcacgacggcacctgcgatatgtcatgacgggaaggcccacttcccgcgggagggagtgttcgtcagcaatggtacccactggtttgtgactcagagaaacttctatgagccacagatcattaccactgacaatacctttgtcagtggtaactgcgacgtggtcataggcatcgtgaacaacacggtgtatgacccgttgcagccggagctcgacagctttaaggaggagctggataagtacttcaagaaccacacctcgccggacgttgatctcggagacatctccgggatcaacgccagcgtggtgaatatacagaaggagattgataggctgaacgaagttgccaagaacctgaacgagtcgctgatagacttgcaggagcttggcaagtacgagcagtatatcaagtggccttggtatatttggctggggtttatagctggtcttatagccatcgtgatggtgaccatcatgttgtgctgtatgaccagctgttgctcctgcctgaagggatgctgttcgtgcgggtcttgctgtaagttcgacgaggacgacagcgagcccgtactgaagggcgtcaagcttcactatacttag

SEQ ID NO: 18 - RNA, 3816 nt, Severe acute respiratory syndrome coronavirus 2
  mRNA 1..3816: note = U indicates pseudouridine (p) or 1-methylpseudouridine (mlf)
  source 1..3816: mol_type = mRNA
atgttcgtgttcctggtgctgctgccgttagtgagtagtcagtgtgtgaacctgactactcgcacgcagctgcctccagcgtacacgaacagctttacgcgcggggtatattaccccgataaggtttttaggtcgagtgtgcttcactcgacccaggacctcttcctcccgttcttctcgaacgtgacgtggttccacgcgatacacgtttcgggaacgaacgggacgaagaggttcgacaaccctgtcttaccgttcaacgatggggtctactttgcctcgacagagaagagtaacatcattcgtggctggatattcgggacaacattggactcgaagacgcagtccttgttgatcgtgaataacgccacgaatgttgttattaaggtctgcgagttccagttctgcaacgaccccttcttggacgtgtactatcacaagaacaacaagtcttggatggagtcgggcgtgtacagctcggctaacaactgtacctttgagtacgtttcgcagccattccttatggacttggaaggtaagcaagggaactttaagaaccttagggagtttgtctttaagaatatcgacggatattttaagatatactccaagcacacaccaatcaatttggtgcgtgatctaccacagggcttcagtgctctggagcccttggtagatctacccattggaataaatatcacccggtttcagacgctcttggccctgcacaggagctatctgactccgggtgatagttccagtgggtggactgcaggtgccgccgcgtactacgtggggtacctgcagccacggacgtttctgttaaagtacaatgagaatggtactataacagacgccgtggactgcgctttggatccgttgtcggagacaaagtgcacgctcaagtcgttcacggtggagaaggggatctaccagactagtaatttccgggtgcagcccacggagagtattgtccggttccccaacatcaccaacctctgtccgttcggcgaggtgttcaacgcgactcggttcgcgagcgtgtacgcctggaatcggaagaggatcagcaactgtgttgctgattacagtgttctgtataactcggcatcctttagcacgttcaagtgctatggggtgtcgcctactaagttgaacgacttgtgctttacgaatgtgtacgcagattcgttcgtgatccggggggacgaggtccgccagatcgcgccaggccagaccggtaagattgccgactacaactacaagctgccagacgatttcacaggctgtgttatagcctggaattcgaacaatctggacagcaaggtaggaggcaattacaattaccggtaccgcctgtttagaaagtccaacttgaagcccttcgagcgggacatttctacggagatatatcaagccggctccaagccgtgcaacggcgtggagggctttaattgttatttcccgctgcagagctacggcttccagccgactaacggggtaggatatcagccctatcgcgttgttgtgctctcgttcgagctgttgcacgccccggccactgtctgtgggcccaagaagtcgacgaacttggtgaagaacaagtgcgtcaacttcaacttcaatgggctcacaggcaccggggtgttaacagagtcgaacaagaagtttcttccattccagcagtttggtcgcgacatcgcggacaccaccgacgcggtgcgcgacccccagacgctggaaatcttggatataactccttgttcgttcgggggcgtgtctgttattacgccggggacgaatacttcgaaccaggtagcggtattgtatcagggggtgaactgtacggaggtgccggtggcgatacatgcggaccagttgacacccacgtggcgggtctatagcacgggcagcaacgtgtttcagacccgcgcggggtgtctaattggtgccgagcatgtaaataactcgtacgagtgcgacatcccgataggggcgggcatttgtgcgagttatcagacccagacgaactcgcctgggtctgcgtcatcggtcgcctcccagagtatcatcgcctatacaatgtcgctaggtgcggagaattctgtcgcctacagtaataacagcatcgccatcccgacgaacttcacgatctcggtgacaaccgagatcttgcctgtcagcatgaccaagacgtcggtcgactgcacgatgtatatatgtggagattctaccgagtgcagcaatcttttgctgcagtacggtagtttctgcacacagttaaatcgtgcgttgaccggcatcgcggtcgagcaggacaagaacacgcaggaggtgttcgcacaggttaagcagatctacaagactcctccgataaaggacttcggggggtttaacttctcgcagatactgccagatccctctaagccctcgaagcgctcctttatcgaggacttgctgtttaacaaggtgacgctggctgatgccggcttcatcaagcagtacggcgactgcttgggggatatagctgcgcgagatctcatctgtgcgcagaagttcaatggactgactgtcctgccccctctgctcacagatgagatgatcgcgcagtatacctccgcgctgcttgccggcaccataacgtccggctggacgtttggtgccggcgcagcgctccagataccgttcgccatgcagatggcgtatcggttcaatggaattggggtgacccaaaacgttctgtacgagaaccagaagctgatcgccaaccagtttaacagcgcgatagggaagattcaggacagcttgagtagcaccgcaagtgcgctgggcaagctgcagaacgtggtgaatcagaacgcgcaggcgctgaacaccctcgttaagcagctgtccagcaacttcggtgctatctcaagcgtcctgaatgatatcctatcccgtctggaccctccggaggcggaggtccagattgatcggctgatcactgggaggctgcagagcctccagacgtacgttacccagcagctcattagagctgtcgagatccggatttccgcaaatctagccgctacgaagatgtccgagtgcgttctcggacagtcgaagcgggtagatttttgcggaaaggggtaccatctgatgagcttcccgcagtccgccccgcatggagtggtcttcctgcatgtcacgtatgtgccggcgcaggagaagaacttcacgacggcacctgcgatatgtcatgacgggaaggcccacttcccgcgggagggagtgttcgtcagcaatggtacccactggtttgtgactcagagaaacttctatgagccacagatcattaccactgacaatacctttgtcagtggtaactgcgacgtggtcataggcatcgtgaacaacacggtgtatgacccgttgcagccggagctcgacagctttaaggaggagctggataagtacttcaagaaccacacctcgccggacgttgatctcggagacatctccgggatcaacgccagcgtggtgaatatacagaaggagattgataggctgaacgaagttgccaagaacctgaacgagtcgctgatagacttgcaggagcttggcaagtacgagcagtatatcaagtggccttggtatatttggctggggtttatagctggtcttatagccatcgtgatggtgaccatcatgttgtgctgtatgaccagctgttgctcctgcctgaagggatgctgttcgtgcgggtcttgctgtaagttcgacgaggacgacagcgagcccgtactgaagggcgtcaagcttcactatacttag

SEQ ID NO: 19 - RNA, 3813 nt, Severe acute respiratory syndrome coronavirus 2
  mRNA 1..3813: note = U indicates pseudouridine (p) or 1-methylpseudouridine (mlf)
  source 1..3813: mol_type = mRNA
atgttcgtgttcctggtgctgctgccgttagtgagtagtcagtgtgtgaacctgactactcgcacgcagctgcctccagcgtacacgaacagctttacccgaggtgtgtactacccggacaaggtgttcaggtcgagtgtgcttcactcgacccaggacctcttcctcccgttcttctccaatgtgacgtggtttcacgtcatatcggggacgaacgggacgaagaggttcgacaaccctgttcttccgttcaacgatggggtctactttgcctcgatagagaagagtaacatcattcgtggctggatattcgggacaacattggactcgaagacgcagtccttgttgatcgtgaataacgccacgaatgtcgttattaaggtctgcgagttccagttctgcaacgaccccttcttggaccacaagaacaacaagagctggatggagtcggagttccgagtctattccagcgctaacaactgcacgttcgagtatgtctcccagccgtttcttatggacttggagggcaagcagggtaacttcaagaatctgcgcgagtttgtgtttaagaatatcgatgggtactttaagatctatagcaagcacactccaatcatcgtgcgggagcccgaggaccttcctcagggcttcagcgcgttggagccgcttgttgatcttcccatcggtataaacataactcgctttcagaccctgcttgccctccatcgttcatatctgacgccgggagacagctcgagcggctggacagctggtgcggcggcgtactacgtcgggtatcttcagccacggacgtttctgttaaagtacaatgagaatggtactataacagacgccgtggactgcgctttggatccgttgtcggagacaaagtgcactctgaagagcttcactgtggagaaggggatctaccagacctctaatttccgcgtgcagcctacagagagtattgttcggtttccgaacattactaacctgtgcccgtttgacgaggtgttcaacgccacgcggttcgcctcggtctatgcgtggaacaggaagcgcatatcgaactgcgtggcggactactcggtactctataaccttgcgccgttctttacgttcaagtgttatggagtatcgccgaccaagctgaacgatttgtgctttacgaatgtgtacgcagattcgtttgtcatccggggggatgaagtccgccagatagctcccgggcagaccgggaatatcgcggactacaattacaagctcccggatgacttcaccgggtgcgtgattgcttggaattcgaacaagttggattccaaggtatcaggtaattacaattacctgtaccggctctttcgtaagagcaacctgaagccgttcgagagagacatctcgacggagatataccaggccgggaacaagccgtgcaacggagtagcaggcttcaactgctactttccgttgcgcagctacagtttccggcctacgtatggagtagggcaccagccctaccgcgtggtggtgctctcctttgagctcctccacgcgccggcgaccgtgtgtgggcccaagaagagcaccaacctggtgaagaataagtgcgtgaactttaacttcaacgggttgaagggtacaggagtgctcaccgagagcaacaagaagttccttcccttccaacagtttggaagggacatagcggacacgacggatgctgtccgggatccccagactctggagatcctggacatcaccccgtgttcgttcgggggcgtcagtgtaatcacccccggtaccaacacgagcaaccaggttgccgtgttgtaccagggggtgaattgcactgaggtccccgttgctatacacgccgaccagttgaccccaacctggcgggtgtatagcacgggctcgaacgtgtttcagacacgtgctgggtgcctgattggcgccgagtacgtgaataactcgtacgagtgcgatataccgattggggctgggatctgcgcgagctatcagacccagacgaagtcgcctgggtctgctagctccgtagcatcccagtccataatcgcgtatactatgagtctcggcgcggagaactcagtggcctatagtaacaactctatagccattccgacgaacttcaccatctcggtgacgacggagattctaccggtgtcgatgaccaagacatcggtagactgtactatgtacatatgcggggactcgacggagtgctcgaacttgttgttgcagtacgggagcttctgtacccagctcaagcgggccctcacagggatcgccgtcgaacaggacaagaacacgcaggaggtgttcgcgcaggtcaagcagatatacaagacccccccgatcaagtattttggggggtttaatttctcgcagatcctgccggacccctccaagcccagcaagcgtagctttatagaggacctcctcttcaataaagttacgcttgctgacgcgggtttcatcaagcagtacggcgactgcttgggagacatcgcggctcgtgacctgatctgcgcgcagaagttcaaggggctgacggtcctcccgccgttgcttacggacgagatgatcgctcagtatacttctgcgctgctggcaggcacgataacgtcgggctggacctttggtgccggcgcggcgttacagataccgttcgccatgcagatggcgtatcggttcaatggtattggggtgacccaaaacgtcctgtacgagaatcagaagctgatcgcgaatcagttcaattctgcgatcggcaagattcaggactcgctgtcctcaacagcgagtgcgttgggtaagctacaggacgtagttaaccacaacgcccaggcgctgaatacgttggtgaagcagctgagctccaagtttggagctatcagcagcgtcctcaacgacatattcagccgcctggaccctccggaggcggaggtccagatagaccggttgattaccggtcgtctgcagagcctgcagacgtacgtaactcagcagctcatccgagctgttgagatacgtattagtgcaaacttggccgccaccaagatgagcgagtgcgttctggggcagagcaagcgggtggacttctgcggcaaggggtaccacctgatgagctttccccagagcgcaccgcacggtgtggtcttcctgcacgtgacatatgtgccggcgcaggagaagaacttcaccactgcgccggccatatgtcacgacgggaaggcccacttcccccgggagggggtttttgtgtccaacgggacacattggttcgtgactcagcggaacttctatgagccgcagatcatcacgaccgacaacaccttcgtgagcgggaactgcgacgtggtgatagggatcgtgaacaacactgtttacgatcccttgcagccggagctggactcgttcaaggaggagctggacaagtatttcaagaatcacacaagtccagacgtggacttgggtgatatctctggaataaatgccagcgtggtcaatatccagaaggagattgaccgcctcaatgaggttgccaagaacctcaatgagagcttgatcgacttgcaggagttgggcaagtacgagcagtacatcaagtggccctggtacatctggctagggtttattgctggtctgatcgctatcgttatggtcaccatcatgctttgctgcatgaccagctgctgcagctgcctgaaggggtgctgcagctgcgggtcatgctgcaagtttgatgaggacgatagcgagcccgtcttgaagggggtcaagttgcactatacttag

SEQ ID NO: 20 - protein, 26 aa, synthetic construct
  REGION 1..26: note = Foldon
YIPEAPRDGQAYVRKDGEWVLLSTFL

SEQ ID NO: 21 - protein, 15 aa, synthetic construct
  REGION 1..15: note = avitag
GLNDIFEAQKIEWHE

SEQ ID NO: 22 - protein, 5 aa, synthetic construct
  REGION 1..5: note = Linker
GGSGS

SEQ ID NO: 23 - protein, 4 aa, synthetic construct
  REGION 1..4: note = Linker
GSGS

SEQ ID NO: 24 - DNA, 10 bp, synthetic construct
  misc_feature 1..10: note = Downstream restriction site sequence
  source 1..10: mol_type = other DNA
tatcgccacc

SEQ ID NO: 25 - protein, 1223 aa, Severe acute respiratory syndrome coronavirus 2
MDAMKRGLCCVLLLCGAVFVSASVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSPGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGSHHHHHH

SEQ ID NO: 26 - DNA, 3669 bp, Severe acute respiratory syndrome coronavirus 2
  source 1..3669: mol_type = genomic DNA
atggatgcaatgaagagagggctctgctgtgtgctgctgctgtgtggagcagtcttcgtttcggctagcgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgaattccagttctgcaacgaccccttcctggaccacaagaacaacaagagctggatggaaagcgagttccgggtgtactccagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctcgagcccctggtggatctgcccatcggcatcaacatcacccggtttcagacactgctggccctgcacagaagctacctgacacctggcgatagcagctctggatggacagctggcgcggccgcctactatgttggctacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtataacctggcccccttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtctccggcaactacaattacctgtaccggctgtttcggaagtccaatcttaagcccttcgagcgggacatctccaccgagatctatcaggccggcaacaagccttgtaacggcgtggccggcttcaactgctacttcccactgcggtcctactcctttcggcccacatacggcgtgggccaccagccttacagagtggtggtgctgtccttcgagctgctgcatgctcctgccacagtgtgcggccctaagaaaagcaccaatctcgtgaagaacaaatgcgtgaacttcaacttcaacggcctgaagggcaccggcgtgctgacagagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacttggagagtgtactctaccggcagcaatgtgtttcagaccagagccggctgtctgatcggagctgagtacgtgaacaatagctacgagtgcgacatccccatcggcgctggcatctgtgccagctaccagacacagacaaagagccccggcagcgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgtacaatgtatatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgatattgccgccagggatctgatctgcgcccagaagtttaagggactgacagtgctgcctcctctgctgaccgatgagatgatcgcccagtacacaagcgctctgctggccggcacaatcacaagcggctggacatttggagctggcgcagccctgcagatcccctttgctatgcagatggcctaccggttcaacggcatcggagtgacccagaacgtgctgtacgagaaccagaagctgatcgccaaccagttcaactccgccatcggcaagatccaggacagcctgagcagcacagcaagcgccctgggaaagctgcaggacgtggtcaaccacaatgcccaggcactgaacaccctggtcaagcagctgtctagcaagttcggcgccatcagctctgtgctgaatgatatcttttccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggagacatctccggaatcaatgcctccgtcgtgaacatccagaaagagatcgaccggctgaacgaggtggccaagaatctgaacgagagcctgatcgacctgcaagaactggggaagtacgagcaaggcagcggatcccaccaccatcatcaccat